(19) United States
(12) Patent Application Publication
(10) Pub. No.: US 2025/0115649 A1
(43) Pub. Date: Apr. 10, 2025

(54) COMPOSITIONS AND METHODS FOR TREATING OR PREVENTING FIBROSIS

(71) Applicant: Nexel Co., Ltd., Seoul (KR)

(72) Inventors: Choongseong Han, Seoul (KR); Dong-Hun Woo, Seoul (KR); Xiong Jin, Seoul (KR); Jaehun Lee, Seoul (KR); Geun Ho An, Seoul (KR); Tack Jin Yoo, Seoul (KR); Jinwoo Chung, Seoul (KR)

(21) Appl. No.: 18/925,356

(22) Filed: Oct. 24, 2024

Related U.S. Application Data
(63) Continuation of application No. 17/285,040, filed on Apr. 13, 2021, now Pat. No. 12,139,518.

(30) Foreign Application Priority Data
Oct. 25, 2018 (KR) 10-2018-0128033
Oct. 25, 2018 (KR) 10-2018-0128204
Oct. 26, 2018 (KR) 10-2018--0128625

(51) Int. Cl. A61K 38/18; A61P 9/10; A61P 11/00; A61P 25/28

(52) U.S. Cl. CPC C07K 14/47; A61P 1/16; A61P 9/00; A61P 11/00; A61P 25/28; C12N 15/815; A61K 38/00; C07K 2319/21; C07K 2319/43

(57) ABSTRACT

The present application relates to compositions and methods comprising polypeptides derived from MFG-E8 for treating and preventing fibrosis and other diseases.

### CROSS-REFERENCE TO RELATED APPLICATIONS

This application claims the benefit of Korean Application No. 10-2018-0128033/KR, filed on Oct. 25, 2018; Korean Application No. 10-2018-0128625/KR, filed on Oct. 26, 2018; and Korean Application No. 10-2018-0128204/KR, filed on Oct. 25, 2018; the entire contents of each of said applications are incorporated herein in their entirety by this reference.

### BACKGROUND OF THE INVENTION

The process of tissue repair as a part of would healing involves two phases. The first phase is the regenerative phase, in which injured cells are replaced by cells of the same type. The second phase is the formation of fibrous tissues, also called fibroplasia or fibrosis, in which connective tissue replaces normal parenchymal tissues. The tissue repair process can become pathogenic if the firbrosis phase continues unchecked, leading to extensive tissue remodeling and the formation of permanent scar tissue.

Liver fibrosis is characterized by an excessive accumulation of extracellular matrix proteins including collagen. In the progression of liver fibrosis, damaged and dead hepatocytes from liver injuries recruit Kupffer cells at their lesion sites. These Kupffer cells secrete substantial quantities of cytokines, including transforming growth factor β1 (TGF-β1) to control liver inflammation. The elevated TGF-β1 causes the activation of quiescent hepatic stellate cells (HSCs) that proliferate and become extracellular matrix (ECM)-producing myofibroblast-like cells. The activated HSCs further accumulate excessive collagen-rich ECM in the liver, leading to contortions in normal liver architecture.

Liver fibrosis results from chronic inflammatory liver diseases or iterative liver damages. Causes for iterative liver injury and fibrosis include viral infections (hepatitis B and C), alcohol abuse, and nonalcoholic steatohepatitis (NASH). Liver fibrosis has the potential to develop into liver cirrhosis and cancer with higher mortality rates than those of other major cancers (lung, colorectal, stomach, or breast cancer). Accordingly, liver fibrosis remains a major cause of death with few therapeutic strategies, and there is a great need for treatment to reduce and prevent fibrosis.

### SUMMARY OF THE INVENTION

The present invention is based, at least in part, on the discovery that a fragment of MFG-E8, hereinafter NP-011, is surprisingly effective in treating and/or preventing diseases including but not limited to fibrosis, cirrhosis, steatosis, and nonalcoholic steatohepatitis (NASH).

In certain aspects, provided herein is a polypeptide for treating and/or preventing diseases including but not limited to fibrosis, cirrhosis, steatosis, and NASH. In some embodiments, the polypeptide comprises an MFG-E8 polypeptide comprising an epidermal growth factor (EGF)-like domain, a C1 domain, and optionally a signal peptide, but lacking a functional C2 domain. In some embodiments, the polypeptide comprises an MFG-E8 polypeptide comprising an epidermal growth factor (EGF)-like domain, a Cl domain, and optionally a signal peptide, but lacking a medin polypeptide or a fragment thereof.

In certain embodiments, said MFG-E8 polypeptide lacks at least 1, 2, 3, 4, 5, 10, 15, 20, 25, 30, 35, 40, 45, 50, 55, 60, 65, 70, 75, 80, 85, 90, 95, 100, or 105 amino acids within the C-terminal domain comprising amino acids 226-335 of the MFG-E8 polypeptide. In some embodiments, the MFG-E8 polypeptide comprises at least 180, 190, 200, 210, 215, 220, 225, 230, 235, 240, 245, 250, 255, 260, 265, 270, 275, or 280 amino acids of the MFG-E8 polypeptide. In some embodiments, the MFG-E8 polypeptide comprises at least 75%, 80%, 85%, 86%, 87%, 88%, 89%, 90%, 91%, 92%, 93%, 94%, 95%, 96%, 97%, 98%, 99%, or 100% of the amino acids 1-225 of SEQ ID NO: 10 or SEQ ID NO: 12. In some embodiments, the MFG-E8 polypeptide comprises at least 75%, 80%, 85%, 86%, 87%, 88%, 89%, 90%, 91%, 92%, 93%, 94%, 95%, 96%, 97%, 98%, 99%, or 100% of the amino acids 24-225 of SEQ ID NO: 10 or SEQ ID NO: 12.

In preferred embodiments, the MFG-E8 polypeptide is not glycosylated.

In certain embodiments, said polypeptide further comprises a heterologous sequence, e.g., a FLAG tag, a HIS tag, and/or a Fc portion of an immunoglobulin. Such a heterologous sequence may extend the half life of the polypeptide in vivo.

In some embodiments, the polypeptide is capable of one or more biological activities including but not limited to: decreasing the expression level of TGF-β; decreasing TGF-β signaling; decreasing phosphorylation of SMAD, e.g., SMAD2, ERK, and/or any other phosphorylated proteins downstream of TGF-β; increasing NOTCH signaling; decreasing fibrosis-related gene expression including but not limited to Colla1, Colla2, or Acta2; decreasing interaction between TGF-β and one or more integrins, e.g., integrin β3 and/or integrin β5; decreasing proliferation of hepatic stellate cells (HSC); decreasing the expression level of matrix metallopeptidase 2 (MMP2), matrix metallopeptidase 12 (MMP12), TMP2, ERK, and/or SMAD2; increasing collagenase activity; and/or increasing collagen uptake by macrophages.

In some embodiments, the polypeptide is in a pharmaceutical composition comprising the polypeptide and one or more pharmaceutically acceptable carriers and/or diluents.

Also provided herein is a kit comprising the polypeptide.

In certain aspects, provided herein is an isolated nucleic acid molecule encoding said polypeptide. In some embodiments, the nucleic acid comprises at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% of the sequence identity to nucleotides 61-735 of SEQ ID NO: 9 or SEQ ID NO: 11. In some embodiments, the nucleic acid comprises at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% of the sequence identity to nucleotides 130-735 of SEQ ID NO: 9 or SEQ ID NO: 11. Also provided herein is a vector comprising the nucleic acid molecule, optionally wherein the vector is an expression vector or a viral vector. In some embodiments, the nucleic acid molecule is operatively linked to a promoter and/or other regulatory sequences. In some embodiments, the nucleic acid molecule and/or the vector are in a pharmaceutical composition suitable for administering to a subject. Such pharmaceutical composition may comprise one or more agents (e.g., liposomes, polymers) that aid the endocytosis and/or longer half-life in vivo, and/or reduce immune reactions against the composition in the subject.

In certain aspects, provided herein is a viral particle comprising said vector. In some embodiments, the viral particle is AAV. In certain some embodiments, the AAV comprising the viral vector is administered to a subject, e.g., a human, to treat and/or prevent diseases such as fibrosis, cirrhosis, steatosis, NASH, myocardial infarction, lung fibrosis, idiopathic pulmonary fibrosis, and/or Alzheimer's Disease.

In certain aspects, provided herein is a host cell comprising said vector. In some such embodiments, the host cell is a mammalian cell. In other embodiments, the host cell is a yeast cell, e.g., Pichia pastoris.

In certain aspects, provided herein is a method of producing a polypeptide by culturing the host cell in a culture medium, e.g., wherein the host cell secretes the polypeptide into the medium. In some embodiments, the method further comprises isolating the polypeptide from the medium and optionally further purifying the polypeptide to generate substantially pure polypeptide.

In some aspects, provided herein is a method of decreasing or inhibiting fibrosis in a subject by contacting a cell of the subject with the polypeptide. Also provided herein is a method of decreasing or inhibiting proliferation of HSC by contacting the HSC with the polypeptide. Additionally provided herein is a method of decreasing or inhibiting steatosis in a subject by contacting a cell of the subject with the polypeptide. In some embodiments, the subject in these methods is a mammal, such as a rat, mouse, or human, preferably a human.

In certain aspects, provided herein is a method of increasing the activity of a macrophage (e.g., an uptake of collagens or fibrotic tissues) by contacting a macrophage with the polypeptide. Such increase in the activity of a macrophage contributes to reversal of fibrosis in various tissues (e.g., liver, lung, etc.).

In certain aspects, provided herein is a method of treating or preventing a disorder in a subject in need thereof by administering to the subject the polypeptide. In some embodiments, the disorder is fibrosis (chronic or acute), cirrhosis, steatosis, NASH, and/or lung fibrosis.

In some embodiments, the polypeptide of present disclosure treats or prevents idiopathic pulmonary fibrosis (IPF). In addition to reducing or eliminating the fibrotic tissues, the polypeptide may also alter the expression level of IPF biomarkers. In some instances, the polypeptide may decrease the the expression level of at least one biomarker selected from αSMA, collagen (Colla1), TMP2, MMP2, MMP12, phosphorylated ERK, ERK, phosphorylated SMAD2, and SMAD2.

In other embodiments, the polypeptide of present disclosure treats or prevents myocardial infarction. The polypeptide may improve or increase the function of a heart following myocardial infarction, e.g., relative to improvement seen in a corresponding untreated subject. For example, the polypeptide may increase left ventricular ejection fraction and/or fraction shortening. Furthermore, the polypeptide may inhibit or decrease the fibrosis associated with myocardial infarction.

The present invention also contemplates a method of treating or preventing Alzheimer's Disease (AD). The subject of treatment may have a genetic mutation associated with AD. For example, the subject may have (i) at least one mutation in amyloid precursor protein (APP) selected from K670N/M671L, 1716V, and V7171; and/or (ii) at least one mutation in PSEN1 selected from M146L and L286V. The polypeptide of present disclosure may improve the memory loss and/or behaviors associated with Alzheimer's disease. In addition, the polypeptide may alter the physiological and histopathological changes in the brain. For example, the polypeptide may decrease the amount of amyloid plaques, the amount of amyloid beta, the number of microglia, the level of neuroinflammation (or inflammation of brain), and/or the amount of glial fibrillary acidic protein (GFAP) in the brain of the subject, preferably in hippocampus and/or cortex of the brain of the subject.

The subject may also be treated with an additional agent that treats the disorder.

In some embodiments, the method does not induce amyloid formation in the subject.

The polypetide may be administered to the subject via any of various routes, e.g., an intravenous, subcutaneous, intra-arterial, intraperitoneal, or intramuscular route.

In some embodiments, the subject in these methods is a mammal, e.g., a rat, mouse, or human, preferably a human.

### BRIEF DESCRIPTION OF THE DRAWINGS

FIG. 1A-FIG. 1F show that NP-011 inhibits liver fibrosis and advantages of NP-011 for treating liver disease. (FIG. 1A) Overall schematic schedule for testing efficacy of the human recombinant proteins in liver fibrosis model. (FIG. 1B) Representative images of histological analysis (H&E and Sirius red staining) for the liver of normal mice. TAA-induced liver fibrosis model (Sham), and protein (MFG-E8, NP-011, NP-012, and NP-013) administered liver fibrosis model. Scale bar, 200 μm. (FIG. 1C) Comparison of quantitative fibrotic area in the livers of the mice. Bars represent the means+SD from three replicates in each group. *P<0.05, **P<0.01. (FIG. 1D) Comparison of mRNA expression for fibrotic markers (Colla1, Colla2, and Acta 2) in the liver of normal mice, TAA-induced liver fibrosis model (Sham), and protein (MFG-E8, NP-011, NP-012, and NP-013) administered liver fibrosis model. Bars represent the means+SD from three replicates in each group. *P<0.05. **P<0.01. (FIG. 1E) Luciferase reporter assay for confirming TGF-β signaling pathway transcriptional activity from human HEK-293 FT cells after 0.5 h. 1 h. 2 h treatment of NP-011 (500 ng/ml) or NP-013 (500 ng/ml). Bars represent the means+SD from three replicates in each group. *P<0.05. **P<0.01. (FIG. 1F) Gene Set Enrichment Assay (GSEA) data showing the significant enrichment of Notch signaling gene sets in NP-011 administered group, but not in MFG-E8 administered group, compared to Sham group.

FIG. 2A-FIG. 2F show that NP-011 inhibits fibrosis at low dosage. Resolution of fibrosis by NP-011 at low dosage. (FIG. 2A) Overall schematic schedule for testing efficacy of the NP-011 in liver fibrosis model. (FIG. 2B) Representative images for comparing fibrotic areas (Sirius red stained areas) in the liver of normal mice, TAA-induced liver fibrosis model and NP-011-administered liver fibrosis model at various dose ranges (20 μg/kg˜160 μg/kg). Scale bar, 200 μm. (FIG. 2C) Quantitative analysis of the fibrotic areas in the livers of tested mice in FIG. 2B. Bars represent the means+SD from four mice in each group. **P<0.01. (FIG. 2D) Comparison of α-SMA (Acta2) expression in the liver of normal mice, TAA-induced liver fibrosis model, and NP-011-administered liver fibrosis model by quantitative PCR and immunostaining, respectively. Scale bar, 100 μm. Bars represent the means+SD from three replicates in each group. **P<0.01. (FIG. 2E) Heatmap data shows fibrosis relevant genes observed in livers from normal mice, TAA-induced mice, and NP-011-administered liver fibrosis mice. (FIG. 2F) Comparison of TGF-β1 mRNA expression in advanced liver fibrosis model. Bars represent the means+SD from three replicates in each group. **P<0.01.

FIG. 3A-FIG. 3E show that NP-011 inhibits and prevents liver fibrosis. Therapeutic efficacy of minimal dosage of NP-011 in various model inductions. (FIG. 3A) Overall schematic schedule for testing efficacy of NP-011 in advanced liver fibrosis model. (FIG. 3B) Representative images for analyzing fibrotic areas in livers of normal mice, TAA-induced liver fibrosis model, and 40 μg/kg NP-011-administered liver fibrosis model with different frequencies of administrations (1, 2, 4, and 6 times). Scale bar, 50 μm. Quantitative analysis of the fibrotic areas for livers of normal mice, TAA-induced liver fibrosis model, and 40 μg/kg NP-011-administered liver fibrosis model presented in left panel. Bars represent the means+SD from four mice in each group. **P<0.01. (FIG. 3C) Overall schematic schedule for testing efficacy of NP-011 in progressing liver fibrosis model. (FIG. 3D) Representative images for analyzing fibrotic areas in livers of normal mice, TAA-induced liver fibrosis model, and TAA-injected liver fibrosis model with co-administrations of NP-011. Scale bar, 100 μm. Quantitative analysis of the fibrotic areas of livers in normal mice, TAA-induced liver fibrosis model, and TAA-injected liver fibrosis model with co-administrations of NP-011. Bars represent the means+SD from five mice in each group. **P<0.01. (FIG. 3E) Left panel: Graphical description represents human liver fibrosis model using human embryonic stem cell (hES)-derived hepatocytes and human primary HSCs treated with APAP. Middle panel: Representative images for analyzing hepatocytes and activated myofibroblasts with ALB (albumin; red) and α-SMA (a biomarker for fibrosis Acta2, green). Scale bar, 50 μm. Right panel: Quantitative analysis of the percentage of α-SMA positive cells in human liver fibrosis model. Bars represent the means+SD from five replicates in each group. **P<0.01.

FIG. 4A-FIG. 4D show that NP-011 inhibits and prevents liver cirrhosis. Efficacy test in DMN-induced cirrohosis model in rat. (FIG. 4A) Overall schematic presentation of efficacy test of NP-011 in DMN-induced liver cirrhosis model. (FIG. 4B) Survival curves of DMN-induced cirrhosis model are shown for 10 rat per group. (FIG. 4C) Representative images for H&E staining and Immunohistochemistry analysis of rat liver tissues labelling alpha smooth muscle actin. (FIG. 4D) The quantification score of the fibrosis areas and both piecemeal and lobular necrosis in the livers of normal, DMN-induced liver cirrhosis model, and NP-011 administrated liver cirrhosis model, and quantification of the positive areas of alpha smooth muscle actin.

FIG. 5A-FIG. 5D show that NP-011 inhibits and prevents NASH. Efficacy test in MCD diet NASH model. (FIG. 5A) Overall schematic presentation of efficacy test of NP-011 in MCD diet NASH model. (FIG. 5B) Representative images for Oil red O staining and (FIG. 5C) the quantification of the relative Oil red O positive areas in the liver of normal mice, MCD diet NASH model, and NP-011 administrated liver NASH model show the significant therapeutic efficacy of secretory NP-011. (FIG. 5D) Quantification of the degree of macrovesicular steatosis and microvesicular steatosis and hypertrophy in the livers of each group. Scale bar, 100 μm. Bars represent the means+SD from five mice in each group. **P<0.01. ANOVA followed by Tukey's multiple comparison test.

FIG. 6 shows the interaction of NP-011 with activated HSCs and macrophages. Representative images of immunostaining for human MFG-E8 (NP-011), mouse α-SMA, and mouse F4/80) (a marker for macrophages) in the liver tissues of NP-011 administrated TAA-induced liver fibrosis model. Scale bar=20 μm.

FIG. 7A-FIG. 7D show that NP-011 inhibits proliferation of HSCs. Actions of NP-011 on HSCs. (FIG. 7A and FIG. 7B) PLA assay for studying physical interaction between TGFBRI and integrin αvβ3/αvβ5. Red signals (white arrow heads) indicates the interaction between TGFBRI and Integrin β3 (FIG. 7A), and β5 (FIG. 7B). Quantitative analysis of the number of red signals in each cell. Bars represent the means+SD from five replicates in each group. *P<0.05, **P<0.01. (FIG. 7C) Western blot (WB) and immunoprecipitation (IP) analysis of the regulation of TGF-signaling in human HSCs and physical interaction between TGFBRI and Integrin β3/β5 in the presence of TGF-β1 (10 ng/ml) and/or NP-011 (500 ng/ml). (FIG. 7D) EdU incorporation assay for cell proliferation of human HSCs in the presence of TGF-β1. NP-011, and/or cilengitide trifluoroacetate (CT, inhibitor of integrin avβ3 and avβ5). Bars represent the means+SD from five replicates in each group. **P<0.01.

FIG. 8A-FIG. 8C show that NP-011 decreases MMP2 expression and increases collagenase activity. Regulating pro-fobrotic MMP2 and collagenase activity in HSCs. (FIG. 7A) Comparison of MMP2 mRNA expression in human HSCs in the presence of TGF-β1. NP-011. Bars represent the means+SD from three replicates in each group. **P<0.01. (FIG. 7B and FIG. 7C) Collagenase activity assay of cell lysates (FIG. 7B) and conditioned media (FIG. 7C) from the culture of control HSCs and TGF-β1-treated HSCs with/without NP-011 treatment. Bars represent the means+SD from three replicates in each group. **P<0.01.

FIG. 9A-FIG. 9E show that NP-011 increases macrophage-mediated collagen uptake. (FIG. 9A) Overall schematic schedule for macrophage differentiation from monocyte by treatment of PMA. (FIG. 9B) Representative images for differentiated macrophages (upper panel) and FITC-labelled beads uptake by differentiated macrophages (lower panel). Scale bar=10 μm (FIG. 9C) Flow cytometry results showed that the FITC beads were uptaken by differentiated macrophages. (FIG. 9D) Graphical description of fibrotic collagen uptake assay by differentiated macrophages with/without NP-011. (FIG. 9E) Quantitative analysis of the relative CUI (collagen uptake index) for control and NP-011 treatment. Bars represent the means+SD from three replicates in each group. **P<0.01.

FIG. 10 shows that NP-011 interacts with activated HSCs and macrophages. Representative images of immunostaining for mouse collagen and mouse F4/80 (a marker for macrophages) in the liver tissues of TAA-induced liver fibrosis model and NP-011 administrated TAA-induced liver fibrosis model. Arrow heads indicates the engulfment of collagen by macrophage (F4/80)-positive cells).

FIG. 11A-FIG. 11C show biodistribution and safety profile of NP-011. (FIG. 11A) The organ distribution in healthy male mice at 30 minutes and 60) minutes after intravenously injection of NP-011 (160 μg/kg). (FIG. 11B and FIG. 11C) Quantitative analysis for the number of inflammatory relevant cells and blood biochemistry in male rat (FIG. 11B) and female rat (FIG. 11C) compared between normal and two NP-011 administration group.

FIG. 12 shows a diagram of a model for idiopathic pulmonary fibrosis (IPF).

FIG. 13 shows the images of lung tissues that were stained to show distribution of collagen and αSMA at day 3 (D3), day 5 (D5), day 7 (D7), and day 14 (D14) after treatment with bleomycin.

FIG. 14 shows the changes in expression levels of IPF biomarkers (Colla1, MMP2, MMP12, and TIMP1) at day 3 (D3), day 5 (D5), day 7 (D7), and day 14 (D14) after treatment with bleomycin.

FIG. 15 shows the changes in expression levels of the subfactors of the signaling pathway related to IPF at at day 3 (D3), day 5 (D5), day 7 (D7), and day 14 (D14) after treatment with bleomycin. The subfactors include αSMA, PERK, tERK, pSMAD2(phosphorylated SMAD2), and tSMAD2 (total SMAD2). Actin is shown as a loading control.

FIG. 16A-FIG. 16B show diagrams of a model for IPF for testing in vivo efficacy of NP-011. Two cases with different time points are presented: Case 1 (FIG. 16A) and Case 2 (FIG. 16B).

FIG. 17 shows that NP-011 is efficacious against IPF. The lung tissues were stained to show distribution of collagen and αSMA after injection of NP-011. Mice were injected with NP-011 at day 3 (Case 1 of FIG. 16A).

FIG. 18 shows that NP-011 is efficacious against IPF. The lung tissues were stained to show distribution of collagen and αSMA after injection of NP-011. Mice were injected with NP-011 at day 5 (Case 2 of FIG. 16B).

FIG. 19 shows that NP-011 is efficacious against IPF. NP-011 reverses the bleomycin-induced changes in expression levels of the subfactors of the signaling pathway related to IPF. Mice were injected with NP-011 at day 3 (Case 1 of FIG. 16A). The subfactors include pERK, tERK, pSMAD2, and tSMAD2. Actin is shown as a loading control. The histogram in the right panel shows quantification of the expression level of pSMAD2 normalized to the actin level.

FIG. 20 shows that NP-011 is efficacious against IPF. NP-011 completely reverses the bleomycin-induced changes in expression levels of the subfactors of the signaling pathway related to IPF. Mice were injected with NP-011 at day 5 (Case 1 of FIG. 16B). The subfactors include αSMA, pERK, and tERK. Actin is shown as a loading control. The histogram in the right panel shows quantification of the expression level of pERK normalized to the actin level.

FIG. 21 shows that NP-011 is efficacious against IPF. NP-011 alters the expression levels of IPF biomarkers (e.g., as shown herein collagen (Cola1), MMP2, MMP12, and TIMP1). Specifically, NP-011 reverses the bleomycin-induced increase in expression levels of IPF biomarks.

FIG. 22 shows a diagram of LAD-ligation model for myocardial infarction. The left anterior descending artery (LAD) of a rat is ligated with one single stitch, forming an ischemia that can be seen almost immediately. By closing the LAD, no further blood flow is permitted in that area, while the surrounding myocardial tissue is nearly not affected. This surgical procedure imitates the pathobiological and pathophysiological aspects occurring in infarction-related myocardial ischemia (e.g., Kolk et al. (2009) J Vis Exp, 21: pii 1438). In addition to the LAD-ligation model, FIG. 22 also presents details of the studies presented in FIG. 23-FIG. 26.

FIG. 23A-FIG. 23C show that NP-011 reverses the symptoms of myocardial infarction, and increases heart function. Measurements of left ventricular ejection fraction (EF) and fraction shortening (FS) are shown for mice at 2 weeks (FIG. 23A), 4 weeks (FIG. 23B), or 8 weeks (FIG. 23C) after LAD ligation. Measurements are shown for mice treated with none (AMI; acute myocardial infarction), PBS (phosphate buffered saline), MFG-E8, or NP-011.

FIG. 24A-FIG. 24B shows that NP-011 reverses symptoms of myocardial infarction, and increases the heart function. Time course analysis of the heart from day 1 to day 56 after LAD ligation is shown. Specifically, functional improvement of heart is assessed by measuring left ventricular ejection fraction (EF; FIG. 24A) and fraction shortening (FS; FIG. 24B). Unlike MFG-E8, NP-011 shows a significant improvement over time in heart function.

FIG. 25 shows a change in body weight after time. No significant change in body weight is observed from day 1 to day 56.

FIG. 26A-FIG. 26B show that NP-011 reverses symptoms of myocardial infarction. NP-011 reverses the fibrosis associated with myocardial infarction. FIG. 26A shows the images of H&E-stained cross-sectioned hearts of rats treated with with none (AMI), PBS, MFG-E8, or NP-011. FIG. 26B shows the measurement of fibrosis area compared to overall cross-sectional area of hears from FIG. 26A.

FIG. 27A-FIG. 27E show that NP-011 reverses the behaviors and memory loss associated with Alzheimer's Disease. FIG. 27A shows a diagram of Alzheimer's Disease (AD) model 5XFAD mice and details of the in vivo studies. 5XFAD mice express human APP and PSEN1 transgenes with a total of five AD-linked mutations: the Swedish (K670N/M671L), Florida (1716V), and London (V7171) mutations in APP, and the M146L and L286V mutations in PSEN1. Behavioral and memory tests were performed after injection of NP-011 to 5XFAD. FIG. 27B shows the behavioral analysis of the AD model immediately after injection with NP-011 (at 3.5 months from the initiation of the study as shown in FIG. 27A). FIG. 27C shows the behavioral analysis of the AD model 3 months after injection with NP-011 (at 6.5 months from the initiation of the study as shown in FIG. 27A). FIG. 27D shows the result of the passive avoidance test of the AD model immediately after injection with NP-011 (at 3.5 months from the initiation of the study as shown in FIG. 27A). FIG. 27E shows the result of the passive avoidance test of the AD model 3 months after injection with NP-011 (at 6.5 months from the initiation of the study as shown in FIG. 27A).

FIG. 28A-FIG. 28B show that NP-011 reverses the neuropathological alterations in the brains of Alzheimer's Disease. Specifically, NP-011 decreases the amount of amyloid plaques in AD. FIG. 28A shows hippocampus and cortex of AD brains stained with thioflavin S that detects amyloid plaques, and FIG. 28B shows quantification of the amyloid plaques as stained by thioflavin S.

FIG. 29A-FIG. 29B show that NP-011 decreases the amount of amyloid beta in Alzheimer's Disease. FIG. 29A shows hippocampus and cortex of AD brains stained with the 6E10 antibody that specifically recognizes amyloid beta, and FIG. 29B shows quantification of the amyloid beta in hippocampus and cortex of AD brains.

FIG. 30A-FIG. 30B show that NP-011 decreases the number of microglial cells in Alzheimer's Disease. FIG. 30A shows hippocampus and cortex of AD brains stained with an IBA-1 antibody that recognizes microglia, and FIG. 30B shows quantification of the microglial cells in hippocampus and cortex of AD brains. FIG. 31A-FIG. 31B show that NP-011 decreases the amount of glial fibrillary acidic protein (GFAP) that is upregulated in astrocytes of Alzheimer's Disease. FIG. 31A shows hippocampus and cortex of AD brains stained for GFAP, and FIG. 30B shows quantification of GFAP in hippocampus and cortex of AD brains.

### DETAILED DESCRIPTION OF THE INVENTION

The present invention relates, in part, to compositions and methods for treating and/or preventing diseases related to fibrosis.

### Definitions

Unless defined otherwise, all technical and scientific terms used herein have the same meaning as commonly understood by one of ordinary skill in the art to which this disclosure is related. The headings provided herein are not limitations of the various embodiments, which can be had by reference to the specification as a whole. Moreover, the terms defined immediately below are more fully defined by reference to the specification in its entirety.

The articles “a” and “an” are used herein to refer to one or to more than one (i.e. to at least one) of the grammatical object of the article. By way of example, “an” element means one element or more than one element.

The terms “about” and “approximately” as used in connection with a numerical value throughout the specification and the claims denotes an interval of accuracy, familiar and acceptable to a person skilled in the art. In general, such interval of accuracy is ±10%. Alternatively, and particularly in biological systems, the terms “about” and “approximately” may mean values that are within an order of magnitude, preferably ≤5-fold and more preferably ≤2-fold of a given value.

The term “coding region” refers to regions of a nucleotide sequence comprising codons which are translated into amino acid residues, whereas the term “noncoding region” refers to regions of a nucleotide sequence that are not translated into amino acids (e.g., 5′ and 3′ untranslated regions).

The term “complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal), then the treatment is prophylactic (i.e., it protects the host against developing the unwanted condition); whereas, if it is administered after manifestation of the unwanted condition, the treatment is therapeutic (i.e., it is intended to diminish, ameliorate, or stabilize the existing unwanted condition or side effects thereof).

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

The amount (e.g., of expression of TGF-β, proliferation of HSC, NOTCH signaling) is “higher” or “lower” or “increased” or “decreased” than the amount of the control, if the amount is greater or less, respectively, than the control level by an amount greater than the standard error of the assay employed to assess amount, and preferably at least 5%, 10%, 20%, 30%, 40%, 50%, 60%, 70%, 80%, 90%, 100%, 150%, 200%, 300%, 350%, 400%, 500%, 600%, 700%, 800%, 900%, 1000% or than that amount. Such “significance” can be assessed from any desired or known point of comparison, such as a particular post-treatment versus pre-treatment measurement ratio (e.g., 1-fold, 1.1-fold, 1.2-fold, 1.3-fold, 1.4-fold, 1.5-fold, and the like). Alternately, the amount of the growth factor in the subject can be considered “significantly” higher or lower than the control amount if the amount is at least about two, three, four, or five times, higher or lower, respectively, than the normal amount of the biomarker. Such “significance” can also be applied to any other measured parameter described herein.

A “therapeutically effective amount” of e.g., a polypeptide is an amount capable of producing a medically desirable result in a treated patient, e.g., decrease in fibrosis, steatosis, decrease in expression of TGF-β, with an acceptable benefit: risk ratio, preferably in a human or non-human mammal.

The term “subject” refer to any subject, particularly a mammalian subject, for whom diagnosis, prognosis, or therapy is desired. Mammalian subjects include but are not limited to humans, non-human primates, domestic animals, farm animals, rodents, and the like, which is to be the recipient of a particular treatment.

By “increase in serum half life” or “increase in half life in vivo” means the positive change in circulating half-life of a modified biologically active molecule relative to its non-modified form. In some embodiments, serum half-life is measured by taking blood samples at various time points after administration of the biologically active molecule, and determining the concentration of that molecule in each sample. Measuring the change in serum concentration with time allows calculation of the serum half-life. By comparing the serum half-life of a modified molecule, e.g., conjugated molecule, with an unmodified molecule, the relative increase in serum half-life or t_{1/2} may be determined.

A “kit” is any manufacture (e.g., a package or container) comprising at least one reagent, e.g., a polypeptide described herein. The kit may also comprise additional reagents, carriers, or diluents. The kit may be promoted, distributed, or sold as a unit for performing the methods of the present invention. The kit may comprise one or more reagents necessary to express a composition useful in the methods of the present invention. In certain embodiments, the kit may further comprise a reference standard. One skilled in the art can envision many such control. Reagents in the kit may be provided in individual containers or as mixtures of two or more reagents in a single container. In addition, instructional materials which describe the use of the compositions within the kit can be included. Numeric ranges are inclusive of the numbers defining the range.

### Polypeptides and Representative Biological Activities

The polypeptides provided herein for treatment of various diseases include a MFG-E8 polypeptide comprising an epidermal growth factor (EGF)-like domain, a C1 domain, and optionally a signal peptide, but lacking a functional C2 domain and/or a medin polypeptide or a fragment thereof. The MFG-E8 polypeptide may lack at least 1, 2, 3, 4, 5, 10, 15, 20, 25, 30, 35, 40, 45, 50, 55, 60, 65, 70, 75, 80, 85, 90, 95, 100, or 105 amino acids within the C-terminal domain of MFG-E8 comprising amino acids 226-335. The MFG-E8 polypeptide may comprise at least 180, 190, 200, 210, 215, 220, 225, 230, 235, 240, 245, 250, 255, 260, 265, 270, 275, or 280 amino acids. The MFG-E8 polypeptide may have at least about 75%, 80%, 85%, 86%, 87%, 88%, 89%, 90%, 91%, 92%, 93%, 94%, 95%, 96%, 97%, 98%, 99%, or 100% sequence identity to the amino acids 1-225 of SEQ ID NO: 10. The polypeptide may also have at least about 75%, 80%, 85%, 86%, 87%, 88%, 89%, 90%, 91%, 92%, 93%, 94%, 95%, 96%, 97%, 98%, 99%, or 100% sequence identity to the amino acids 2-225 of SEQ ID NO: 10. The MFG-E8 polypeptide may be of human or of any orthologs, e.g., mouse, rat, chimpanzee, horse, etc.

MFG-E8 is also called lactadherin. Representative human MFG-E8 cDNA and human BRD7 protein sequences are well-known in the art and are publicly available from the National Center for Biotechnology Information (NCBI). For example, four different human MFG-E8 isoforms are known. Human MFG-E8 isoform B (NP_001108086.1) is encodable by the transcript variant 2 (NM_001114614.3), which lacks an alternate in-frame exon compared to variant 1. The resulting isoform B has the same N- and C-termini but is shorter compared to isoform A. Human MFG-E8 isoform D (NP_001297248.1) is encodable by transcript variant 4 (NM_001310319.2), which lacks an alternate in-frame exon in the 5′ coding region compared to transcript variant 1. The encoded isoform D has the same N- and C-termini but is shorter compared to isoform A. Human MFG-E8 isoform C (NP_001297249.1) is encodable by the transcript variant 3 (NM_001310320.2), which includes an alternate internal exon resulting in the use of an alternate translation start site, compared to variant 1. The encoded isoform C has a shorter distinct N-terminus compared to isoform A. Human MFG-E8 isoform E (NP_001297250.1) is encodable by the transcript variant 5 (NM_001310321.2), which includes an alternate 5′ terminal exon resulting in the use of a downstream in-frame translation start site, compared to variant 1. The encoded isoform E has a shorter N-terminus than isoform A. Human MFG-E8 isoform A (NP_005919.2) is encodable by the transcript variant 1 (NM_005928.4), which encodes the longest isoform A. Nucleic acid and polypeptide sequences of MFG-E8 orthologs in organisms other than human are well know and include for example, chimpanzee MFG-E8 (XM_016927386.2→XP_016782875.1; and XM_001165898.6→XP_001165898.1), dog MFG-E8 (XM_022416924.1→XP_022272632.1), horse MFG-E8 (XM_023650241.1→XP_023506009.1; XM_023650222.1→XP_023505990.1; and XM_023650232.1→XP_023506000.1), pig MFG-E8 (XM_021098306.1→XP_020953965.1; and XM_013996945.2→XP_013852399.1), cat MFG-E8 (XM_011282749.2→XP_011281051.1; and XM_011282750.2→XP_011281052.1), sheep MFG-E8 (XM_027957063.1 4→XP_027812864.1), goat MFG-E8 (XM_018065819.1→XP_017921308.1), rat MFG-E8 (NM_001040186.2→NP_001035276.1; and NM_012811.3→NP_036943.1), and mouse MFG-E8 (NM_001045489.1→NP_001038954.1; and NM_008594.2→NP_032620.2).

A representative polypeptide is NP-011. NP-011 and other polypeptides described herein have many biological activities that are useful in inhibiting fibrosis. In some embodiments, polypeptides, such as NP-011, described herein decrease the expression of many genes including TGF-β1, MMP2, and/or fibrosis-related genes, e.g., Colla1, Colla2, Acta2.

In some embodiments, the polypeptides described herein modulate the signaling pathway, e.g., decreases TGF-β signaling or increases NOTCH signaling. TGF-β signaling controls a diverse set of cellular processes, including cell proliferation, recognition, differentiation, apoptosis, and specification of developmental fate, during embryogenesis as well as in mature tissues, in species ranging from flies and worms to mammals. A TGF-β ligand initiates signaling by binding to and bringing together type I and type II receptor serine/threonine kinases on the cell surface. This allows receptor II to phosphorylate the receptor I kinase domain, which then propagates the signal through phosphorylation of the Smad proteins. The activated Smad complexes are translocated into the nucleus and, in conjunction with other nuclear cofactors, regulate the transcription of target genes.

TGF-β and its signaling pathway regulate cell proliferation. In the progression of liver fibrosis, damaged and dead hepatocytes from liver injuries recruit Kupffer cells at their lesion sites. These Kupffer cells secrete substantial quantities of cytokines, including transforming growth factor β1 (TGF-β1) to control liver inflammation. The elevated TGF-β1 causes the activation of quiescent HSCs that proliferate and become extracellular matrix (ECM)-producing myofibroblast-like cells. The activated HSCs further accumulate excessive collagen-rich ECM in the liver, leading to contortions in normal liver architecture. Accordingly, decreasing the expression of TGF-β1 or decreasing TGF-β signaling inhibits activation and/or proliferation of HSCs, and results in inhibition of fibrosis. Similarly, NOTCH signaling is important for liver regeneration, and increase in NOTCH signaling demonstrates NP-011's role in promoting liver regeneration.

Decrease in TGF-B signaling may be monitored via many ways known in the art. For example, the level of expression of downstream target genes (e.g., ATF4, CDKN1A (p21CIPI, WAF1), CDKN1B (P27KIPI), CDKN2B (p15INK4b), COL1A1, COL1A2, DCN, EMP1, FOS, GADD45B, GSC, HERPUD1, IFRD1, IGF1, IGFBP3, IL6, JUN, JUNB, MYC, PDGFB, SERPINE1 (PAI-1), TGFB1I1, TNFSF10) (TRAIL), TSC22D1 (TGFB1I4), TGFB1, TGIF1) and/or phosphorylation of downstream proteins (e.g., SMAD) may be monitored. In addition, there are many commercially available kits that allow monitoring of the TGF-β signaling pathway, e.g., Qiagen Cat #PAHS-035Z, PAMM-035Z, PARN-035Z, PAHS-235Z, PAMM-235Z, PARN-235Z, CRHS-00035Z-100, CRHS-00245Z-100, CRMM-00035Z-100, CRMM-00235Z-100, CCS-017L, CCS-017G, CLS-017L, EAHS-251Z, GH-035A, SEH00508A, SEM02991A. Similarly, the level of expression of downstream target genes (e.g., CDKN1A (p21CIP1, WAF1), CFLAR (Casper), FOSL1 (fra-1), ID1, IL2RA (CD25), NFKB1, PTCRA, CD44, ERBB2 (HER-2, NEU), DTX1, HES1, HESS, HEY1, HEY2, HEYL, JAG1, KRT1, LFNG, LOR, NOTCH1, PPARG, CHUK (IKKα), IFNG, IL17B, IL2RA (CD25), NFKB1, NFKB2, STAT6) and/or phosphorylation of the downstream protein may be monitored to assess the Notch signaling pathway. For example, there are many commercially available kits that allow monitoring of the Notch signaling pathway, e.g., Qiagen Cat #PAHS-059Z, PAMM-059Z, PARN-059Z, CRHS-30 00059Z-100, CRMM-00059Z-100, CCS-014L, CCS-1014G, CLS-014L, CLS-014G, EAHS-611Z, GH-059A.

In some embodiments, as described above, the polypeptides described herein decrease the phosphorylation of SMAD, a protein in the TGF-β signaling pathway.

In some embodiments, the polypeptides described herein disrupt and/or decrease interaction between TGF-β receptor 1 (TGFBR1) and one or more integrins, e.g., integrin β3 and/or integrin β5.

In some embodiments, the polypeptides described herein decreases proliferation of HSCs.

In some embodiments, the polypeptides described herein increases collagenase activity and/or collagen uptake by macrophages.

### Amyloid Formation

Certain polypeptides provided herein lack a medin polypeptide or a fragment thereof, which represents about 50 amino acids of the C2 domain. Medin is a major constituent of amyloid found in the aorta. Medin and the amyloid have been implicated in etiology of diseases including Alzheimer's disease and type 2 diabetes. Repeated administration of a polypeptide comprising the medin polypeptide may increase the risk of inducing amyloid formation. The polypeptides provided herein that lack the medin polypeptide or a fragment thereof reduce the risk of inducing amyloid formation.

### Modification of a Polypeptide

The present invention further provides a modified polypeptide wherein the polypeptide described herein is fused in frame with a heterologous polypeptide and/or one or more chemical moieties. Such modification may be useful, for example, in extending the serum half-life of the polypeptide, serving as a tag during protein purification, or enhancing immunogenicity during production of antibodies to the polypeptide.

As used herein, a “chimeric protein” or “fusion protein” comprises all or part (preferably a biologically active part) of a polypeptide of the present invention linked to a heterologous polypeptide having an amino acid sequence corresponding to a protein which is not substantially homologous to the respective polypeptide. Within the fusion protein, the polypeptide of the present invention and the heterologous polypeptide are preferably fused to each other in such a way as to preserve their respective functions exhibited when expressed independently of the fusion. The heterologous polypeptide can be fused to the amino-terminus or the carboxyl-terminus of the polypeptide of the present invention.

In some embodiments, the fusion protein contains a heterologous signal sequence, immunoglobulin fusion protein, tag (e.g., FLAG, GST, etc.), toxin, or other useful protein sequence. Chimeric and fusion proteins of the present invention can be produced by standard recombinant DNA techniques. In other embodiments, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence. Moreover, many expression vectors are commercially available that already encode a fusion moiety (e.g., a GST polypeptide). A nucleic acid encoding a polypeptide of the present invention can be cloned into such an expression vector so that the fusion moiety is linked in-frame to the polypeptide of the present invention.

The heterologous peptide may optionally correspond to a moiety that alters the solubility, affinity, stability or valency of the polypeptide (e.g., NP-011). For example, a polypeptide such as NP-011 may be operably linked to an immunoglobulin constant region such as a human Cγ1 domain or Cγ4 domain (e.g., the hinge, CH2 and CH3 regions of human IgCγ1, or human IgCγ4, see e.g., Capon et al. U.S. Pat. Nos. 5,116,964; 5,580,756; 5,844,095 and the like, incorporated herein by reference). Such constant regions may retain regions which mediate effector function (e.g., Fc receptor binding) or may be altered to reduce effector function. Such constant regions may also extend the serum half-life of the fusion protein. A resulting fusion protein may have altered solubility, binding affinity, stability and/or valency (i.e., the number of binding sites available per polypeptide) as compared to the independently expressed first peptide, and may increase the efficiency of protein purification. In preferred embodiments, the fusion protein retains the biological activity of the polypeptide.

In other embodiments, a polypeptide may be modified covalently or non-covalently with one or more chemical moieties, e.g., polyethylene glycol (PEG), lipids, or PEG-modified lipids. Such modification, for example, may increase the solubility, stability, and/or serum-half life of the polypeptide.

### Therapeutic Methods-Exemplary Conditions

The compositions and methods provided herein are particularly useful in treating and/or preventing the disorders including those presented below.

### Liver Fibrosis

In hepatic fibrosis, excessive connective tissue accumulates in the liver; this tissue represents scarring in response to chronic, repeated liver cell injury. Commonly, fibrosis progresses, disrupting hepatic architecture and eventually function, as regenerating hepatocytes attempt to replace and repair damaged tissue. When such disruption is widespread, cirrhosis is diagnosed.

Various types of chronic liver injury can cause fibrosis. Self-limited, acute liver injury (e.g., acute viral hepatitis A), even when fulminant, does not necessarily distort the scaffolding architecture and hence does not cause fibrosis, despite loss of hepatocytes. In its initial stages, hepatic fibrosis can regress if the cause is reversible (eg, with viral clearance). After months or years of chronic or repeated injury, fibrosis becomes permanent. Fibrosis develops even more rapidly in mechanical biliary obstruction.

Activation of the hepatic perivascular stellate cells (Ito cells, which store fat) initiates fibrosis. These and adjacent cells proliferate, becoming contractile cells termed myofibroblasts. These cells produce excessive amounts of abnormal matrix (consisting of collagen, other glycoproteins, and glycans) and matricellular proteins. Kupffer cells (resident macrophages), injured hepatocytes, platelets, and leukocytes aggregate. As a result, reactive oxygen species and inflammatory mediators (e.g., platelet-derived growth factor, transforming growth factors, connective tissue growth factor) are released. Thus, stellate cell activation results in abnormal extracellular matrix, both in quantity and composition.

Myofibroblasts, stimulated by endothelin-1, contribute to increased portal vein resistance and increase the density of the abnormal matrix. Fibrous tracts join branches of afferent portal veins and efferent hepatic veins, bypassing the hepatocytes and limiting their blood supply. Hence, fibrosis contributes both to hepatocyte ischemia (causing hepatocellular dysfunction) and portal hypertension. The extent of the ischemia and portal hypertension determines how the liver is affected. For example, congenital hepatic fibrosis affects portal vein branches, largely sparing the parenchyma. The result is portal hypertension with sparing of hepatocellular function.

### Cirrhosis

Cirrhosis is a late stage of hepatic fibrosis that has resulted in widespread distortion of normal hepatic architecture. Cirrhosis is characterized by regenerative nodules surrounded by dense fibrotic tissue. Symptoms may not develop for years and are often nonspecific (e.g., anorexia, fatigue, weight loss). Late manifestations include portal hypertension, ascites, and, when decompensation occurs, liver failure. Diagnosis often requires liver biopsy. Cirrhosis is usually considered irreversible. Treatment is supportive.

Cirrhosis is the seventh leading cause of death in the United States, according to the National Institute of Diabetes and Digestive and Kidney Diseases (NIDDK). Cirrhosis is defined pathologically by the loss of normal microscopic lobular architecture with fibrosis (i.e., the growth of scar tissue due to infection, inflammation, injury, or even healing) and nodular regeneration. Because of chronic damage to the liver, scar tissue slowly replaces normal functioning liver tissue resulting in progressively diminishing blood flow through the liver. As the normal liver tissue is lost, nutrients, hormones, drugs and poisons are not processed effectively by the liver. In addition, protein production and other substances produced by the liver are inhibited.

Symptoms of cirrhosis vary, depending on severity and individuals. Symptoms may include abnormal nerve function, ascites (build-up of fluid in the abdominal cavity), breast enlargement in men, coughing up or vomiting blood, curling of fingers (Dupuytren contracture of the palms), gallstones, hair loss, itching, jaundice, kidney failure, liver encephalopathy, muscle loss, poor appetite, portal hypertension, redness of palms, salivary gland enlargement in cheeks, shrinking of testes, small spider-like veins in skin, weakness, weight loss, etc. The symptoms of cirrhosis may resemble other conditions or medical problems. Mild cirrhosis may not exhibit any symptoms at all.

The most common cause of cirrhosis is alcohol abuse. Other causes include hepatitis and other viruses (e.g., HCV as described in Section 2.2 infra.), use of certain drugs, chemical exposure, bile duct obstruction, autoimmune diseases, obstruction of outflow of blood from the liver (i.e., Budd-Chiari syndrome), heart and blood vessel disturbances, alpha1-antitrypsin deficiency, high blood galactose levels, high blood tyrosine levels, glycogen storage disease, diabetes, malnutrition, hereditary accumulation of too much copper (Wilson Disease) or iron (hemochromatosis).

Cirrhosis is a progressive liver disease, and the damage sustained by the liver is irreversible. However, with proper nutrition, avoidance of certain toxins (i.e., alcohol), vitamin supplementation, and management of cirrhosis complications, further liver damage can often be delayed or stopped. In severe cases of cirrhosis, liver transplantation may be considered.

### Nonalcoholic Steatohepatitis (NASH)

Non-alcoholic steatohepatitis (NASH), also known as non-alcoholic fatty liver disease, describes a hepatic disorder typically characterized by an alcoholic pathogenesis without alcohol consumption. The fat deposit in liver cells is mostly triglyceride, and the severity of NASH is directly related to the amount of fat in the liver. Histologically, if 50% of liver cells had steatosis (fatty liver accumulation), or if the total weight of fat is greater than 5% of the entire liver, then steatohepatitis can be diagnosed. NASH is further characterized by elevated serum aminotransferase activities with hepatic steatosis, inflammation, and occasionally fibrosis that may progress to cirrhosis.

The prevalence of NASH is 3-19% throughout most of the world. There are many possible causes of NASH but there isn't a definite source. The most likely causes are obesity from poor diet, diabetes, long-term use of steroids and use of tetracycline. Some studies have shown sign of steatosis reversal after weight loss.

There is currently no established treatment that exists for this potentially serious disorder. Treatment of patients with nonalcoholic fatty liver has typically been focused on the management of associated conditions such as obesity, diabetes mellitus, and hyperlipidemia as well as discontinuation of potentially hepatotoxic drugs.

### Fibrosis Related Disorders

Fibrosis related disorders that may be amenable to treatment with the methods provided herein include, but are not limited to, collagen disease, interstitial lung disease, human fibrotic lung disease (e.g., obliterative bronchiolitis, idiopathic pulmonary fibrosis, pulmonary fibrosis from a known etiology, tumor stroma in lung disease, systemic sclerosis affecting the lungs, Hermansky-Pudlak syndrome, coal worker's pneumoconiosis, asbestosis, silicosis, chronic pulmonary hypertension, AIDS-associated pulmonary hypertension, sarcoidosis, moderate to severe asthma and the like), fibrotic vascular disease, arterial sclerosis, atherosclerosis, varicose veins, coronary infarcts, cerebral infarcts, myocardial fibrosis, musculoskeletal fibrosis, post-surgical adhesions, human kidney disease (e.g., nephritic syndrome, Alport syndrome, HIV-associated nephropathy, polycystic kidney disease, Fabry's disease, diabetic nephropathy, chronic glomerulonephritis, nephritis associated with systemic lupus, and the like), progressive systemic sclerosis (PSS), primary sclerosing cholangitis (PSC), liver fibrosis, liver cirrhosis, renal fibrosis, pulmonary fibrosis, cystic fibrosis, chronic graft versus host disease, scleroderma (local and systemic), Grave's ophthalmopathy, diabetic retinopathy, glaucoma, Peyronie's disease, penis fibrosis, urethrostenosis after cystoscope, inner accretion after surgery, scarring, myelofibrosis, idiopathic retroperitoneal fibrosis, peritoneal fibrosis from a known etiology, drug-induced ergotism, fibrosis incident to benign or malignant cancer, fibrosis incident to microbial infection (e.g., viral, bacterial, parasitic, fungal, etc.), Alzheimer's disease, fibrosis incident to inflammatory bowel disease (including stricture formation in Crohn's disease and microscopic colitis), stromal cell tumors, mucositis, fibrosis induced by chemical or environmental insult (e.g., cancer chemotherapy, pesticides, radiation (e.g., cancer radiotherapy), and the like), and the like. In some embodiments, the fibrosis related disorder is selected from systemic or local scleroderma, keloids, hypertrophic scars, atherosclerosis, restenosis, pulmonary inflammation and fibrosis, idiopathic pulmonary fibrosis, liver cirrhosis, fibrosis as a result of chronic hepatitis B or C infection, kidney disease, heart disease resulting from scar tissue, macular degeneration, and retinal and vitreal retinopathy. In some embodiments, the fibrosis related disorder results from chemotherapeutic drugs, radiation-induced fibrosis, and injuries and burns.

Inherited disorders associated with hepatic fibrosis include Wilson's disease, hereditary hemochromatosis, non-HFE hereditary hemochromatosis ferroportin, Transferrin receptor 2, Hepcidin, Hemojuvelin, Sitosterolemia/hepatobiliary cholesterol transporter 5 and 8, progressive familial intrahepatic cholestasis type 3, hereditary fructose intolerance, tyrosinemia type I, argininosuccinate lyase deficiency, citrin deficiency, cholesteryl ester storage disease and Wolman disease, anti-1 antitrypsin deficiency, cystic fibrosis, Alstrom syndrome, and congenital hepatic fibrosis.

### Lung Fibrosis

Lung fibrosis (“scarring of the lungs”) is a respiratory disease in which scars are formed in the lung tissues, leading to serious breathing problems. Scar formation, the accumulation of excess fibrous connective tissue (the process called fibrosis), leads to thickening of the walls, and causes reduced oxygen supply in the blood. A consequence is a perpetual shortness of breath.

Lung fibrosis involves gradual exchange of normal lung parenchyma with fibrotic tissue. The replacement of normal lung with scar tissue causes irreversible decrease in oxygen diffusion capacity, and the resulting stiffness or decreased compliance makes lung fibrosis a restrictive lung disease. Lung fibrosis is perpetuated by aberrant wound healing, rather than chronic inflammation. It is the main cause of restrictive lung disease that is intrinsic to the lung parenchyma. In some cases, lung fibrosis has been associated with cigarette smoking. Treatment option for lung fibrosis is limiting, and includes immunosuppressive therapy such as corticosteroids.

### Idiopathic Pulmonary Fibrosis

Idiopathic pulmonary fibrosis (IPF), the most common form of the idiopathic interstitial pneumonias, is a chronic, progressive, irreversible, and usually lethal lung disease of unknown cause. IPF occurs in middle-aged and elderly adults (median age at diagnosis 66 years, range 55-75 years), is limited to the lungs, and is associated with a histopathological or radiological pattern typical of usual interstitial pneumonia.

The main histopathological features of usual interstitial pneumonia, best seen at low magnification, is a heterogeneous appearance with areas of subpleural and paraseptal fibrosis and honeycombing (i.e., cystic fibrotic airspaces lined by bronchiolar epithelium and often filled by mucin and variable numbers of inflammatory cells) alternating with areas of less affected or normal parenchyma (spatial heterogeneity). Small areas of active fibrosis (fibroblast foci) are present in the background of collagen deposition, and they reflect the temporal heterogeneity of the process and indicate current ongoing disease. Inflammation is usually mild and consists of a patchy lymphoplasmacytic interstitial infiltrate. The presence of a usual-interstitial-pneumonia pattern on high-resolution CT is characterised by reticular opacities, often associated with traction bronchiectasis, with little or no ground-glass opacifications. Honeycombing, manifested as subpleural, clustered cystic airspaces with well-defined walls (typically 3-10 mm in diameter), is common and is critical for making a definite diagnosis.

Patients with IPF usually seek medical attention because they suffer chronic and progressive exertional dyspnoea and cough. Bibasilar inspiratory crackles are heard on chest auscultation and frequently finger clubbing is found. The natural history of IPF has been characterised as a steady or slowly progressive lung disorder, and most patients follow this pattern. However, recent findings indicate that IPF is a heterogeneous disease and new clinical phenotypes with distinct patterns of survival are being described. The pathogenic mechanisms are unclear, but a growing body of evidence indicates that the disease is the result of an abnormal behaviour of the alveolar epithelial cells that provoke the migration, proliferation, and activation of mesenchymal cells, with the formation of fibroblast and myofibroblast foci. Activated myofibroblasts secrete exaggerated amounts of extracellular matrix molecules with the subsequent destruction of the lung architecture. No therapy has been clearly shown to prolong survival.

### Myocardial Infarction

A myocardial infarction, commonly known as a heart attack, occurs when a portion of the heart is deprived of oxygen due to blockage of a coronary artery. Coronary arteries supply the heart muscle (myocardium) with oxygenated blood. Without oxygen, muscle cells served by the blocked artery begin to die (infarct).

The most common cause of a myocardial infarction is the rupture of an atherosclerotic plaque on an artery supplying heart muscle. Plaques can become unstable, rupture, and additionally promote the formation of a blood clot that blocks the artery: this can occur in minutes. Blockage of an artery can lead to tissue death in tissue being supplied by that artery. Atherosclerotic plaques are often present for decades before they result in symptoms.

The gradual buildup of cholesterol and fibrous tissue in plaques in the wall of the coronary arteries or other arteries, typically over decades, is termed atherosclerosis. Atherosclerosis is characterized by progressive inflammation of the walls of the arteries. Inflammatory cells, particularly macrophages, move into affected arterial walls. Over time, they become laden with cholesterol products, particularly LDL, and become foam cells. A cholesterol core forms as foam cells die. In response to growth factors secreted by macrophages, smooth muscle and other cells move into the plaque and act to stabilize it. A stable plaque may have a thick fibrous cap with calcification. If there is ongoing inflammation, the cap may be thin or ulcerate. Exposed to the pressure associated with blood flow, plaques, especially those with a thin lining, may rupture and trigger the formation of a blood clot (thrombus).

### Alzheimer'S Disease

Alzheimer's Disease is a dementing disorder characterized by progressive impairments in memory and cognition. It typically occurs in later life; and is associated with a multiplicity of structural, chemical and functional abnormalities involving brain regions concerned with cognition and memory. Epidemiologic studies suggest that the dementia presently occurs in up to 10% of individuals over the age of 65 and it is estimated that in the United States alone, as many as 4 million individuals may be affected with Alzheimer's Disease. The cost of caring for such individuals is well over 80 billion dollars annually and increasing rapidly.

The cause of Alzheimer's disease is poorly understood. About 70% of the risk is believed to be inherited from a person's parents with many genes usually involved. Other risk factors include a history of head injuries, depression, and hypertension. The disease process is associated with plaques and neurofibrillary tangles in the brain. A probable diagnosis is based on the history of the illness and cognitive testing with medical imaging and blood tests to rule out other possible causes. Initial symptoms are often mistaken for normal ageing. Examination of brain tissue is needed for a definite diagnosis. There are no medications or supplements that have been shown to decrease risk.

It has been postulated that extracellular amyloid beta (A_{β}) deposits are the fundamental cause of the disease. Support for this postulate comes from the location of the gene for the amyloid precursor protein (APP) on chromosome 21, together with the fact that people with trisomy 21 (Down Syndrome) who have an extra gene copy almost universally exhibit at least the earliest symptoms of AD by 40 years of age. Also, a specific isoform of apolipoprotein, APOE4, is a major genetic risk factor for AD. While apolipoproteins enhance the breakdown of beta amyloid, some isoforms are not very effective at this task (such as APOE4), leading to excess amyloid buildup in the brain. Further evidence comes from the finding that transgenic mice that express a mutant form of the human APP gene develop fibrillar amyloid plaques and Alzheimer's-like brain pathology with spatial learning deficits.

### Additional Agents

The compositions provided herein may be administered to a subject in combination with any known agents that treat the disorder, e.g., liver fibrosis. Similarly, the methods provided herein may administer the subject any known agents that treat the disorder in combination with the compositions (e.g., NP-011) disclosed herein. Various treatments for fibrosis-related disorders are known to those skilled in the art.

Treatments for fibrotic disorders include anti-inflammatory agents, corticosteroids, penicillamine, and colchicine. See e.g., The Merck Manual. 20th ed. Merck Research Laboratories, 2018.

Because fibrosis represents a response to hepatic damage, primary treatment should focus on the cause (removing the basis of the liver injury). Such treatment may include eliminating hepatitis B virus or hepatitis C virus in chronic viral hepatitis, abstaining from alcohol in alcoholic liver disease, removing heavy metals such as iron in hemochromatosis or copper in Wilson disease, and decompressing bile ducts in biliary obstruction. Such treatments may stop the fibrosis from progressing and, in some patients, also reverse some of the fibrotic changes.

Treatments aimed at reversing the fibrosis are usually too toxic for long-term use (e.g., corticosteroids, penicillamine) or have no proven efficacy (eg, colchicine). Other antifibrotic treatments are under study. Silymarin, present in milk thistle, is a popular alternative medicine used to treat hepatic fibrosis. It appears to be safe (except when combined with certain drugs to treat hepatitis C) but lacks efficacy.

In some embodiments, anti-fibrotic therapy includes administration of profibrotic factor antagonists and/or anti-fibrotic agents.

### Profibrotic Factor Antagonists

Anti-fibrotic therapy encompasses agents that inhibit or antagonize profibrotic factors, such as agents that antagonize one or more growth factors or cytokines involved in the formation and maintenance of fibrotic tissue. In this manner, anti-fibrotic therapy targets fibrocyte, fibrocyte precursor, myofibroblast precursor, and/or hematopoetic monocyte precursor differentiation and fibrotic tissue formation and maintenance.

Pro fibrotic factors that may be targeted with antagonists as part of the therapies of the present invention include, without limitation, a transforming growth factor type β (TGF-β, including TGF-β1-5), VEGF, EGF, RANTES, members of the interleukin family (e.g., IL-1, IL-4, IL-5, IL-6, IL-8 and IL-13), tumor necrosis factor type alpha (TNF-a), platelet-derived growth factor (PDGF), basic fibroblast growth factor (bFGF), monocyte chemoattractant protein type 1 (MCP-1), macrophage inflammatory protein (e.g., MIP-la, MIP-2), connective tissue growth factor (CTGF), endothelin-1, angiotensin-11, rennin, leptin, chemokines (e.g., CCL2, CCL12, CXCL12, CXCR4, CCR3, CCR5, CCR7), SLC/CCL21 and other factors known to promote or be related to the formation, growth, or maintenance of fibrotic tissue.

Anti-fibrotic therapy may include antagonists of the corresponding receptor of one or more of the pro fibrotic factors. Such antagonists may include inactive forms of one or more of the pro fibrotic factors and/or cytokines, such as fragments thereof. Such forms in suitable concentrations may compete with its corresponding profibrotic factors and/or cytokines for binding to its receptor. Similarly, certain antibodies to the receptor may be used to interfere with or prevent binding thereto of the corresponding pro fibrotic factors and/or cytokines.

Anti-fibrotic therapy may also include soluble forms of the receptor of one or more of the pro fibrotic factors and/or cytokines, such that the soluble receptor competes with its corresponding native cellular receptor for the target ligand. The therapy may further include compounds that compete with or otherwise interfere with binding of one or more of the profibrotic factors and/or cytokines with its receptor. For example, the proteoglycan decorin is known to bind to TGF-β, thereby reducing its availability for binding to its receptor. Mannose-6-phosphate is also known to compete with TGF-β for binding to its corresponding receptor. Other known binding inhibitors of TGF-β include latent transforming growth factor-β binding protein (LTBP) and latency associated peptide (LAP), both of which natively bind to the intracellular precursor of TGF-β.

In certain embodiments, anti-fibrotic therapy may include one or more oligoribonucleotides that contain at least one sequence that is antisense with respect to one or more of the profibrotic factors and/or cytokines. Such components may also include one or more expression plasmids having suitable transcriptional control sequences that yield antisense sequences. In other selected embodiments, anti-fibrotic therapy may include one or more double-stranded oligoribonucleotides, or expression plasmids encoding thereof, that are suitable for degrading transcripts of one or more of the profibrotic factors and/or cytokines via RNAmediated interference. In other selected embodiments, anti-fibrotic therapy may include one or more single-stranded oligonucleotide aptamers, or expression plasmids encoding thereof, that are suitable for inhibiting or interfering with the binding of pro fibrotic factors to their cognate receptors.

A suitable profibrotic factor antagonist may include components known to inhibit, attenuate, or interfere with one or more components of the intracellular signaling pathways activated by one or more of the pro fibrotic factors upon binding to its corresponding receptor. For example, anti-fibrotic therapy may include components that inhibit or attenuate downstream signal pathway molecules such as SMAD family members and SARA. A suitable anti-fibrotic therapy may include one or more molecules that are suitable for inhibiting or interfering with the cellular adhesions require for fibrosis. For example, a suitable component may include interfering antibodies to the ICAM-1 and/or CD 11, CD 49 or CD 18 molecules, thereby interfering with the adhesion interaction there between.

In other selected embodiments, a suitable profibrotic factor antagonist may include inhibitors of collagen synthesis, such as proline analogs that interfere with post-translation processing of collagen precursors. Pirfenidone, for example, is an orally active small molecule drug that may inhibit collagen synthesis, downregulate production of multiple cytokines and block fibroblast proliferation.

### TGF-β Antagonists

Cytokines of the transforming growth factor (TGF) beta family play a central role in wound healing and in tissue repair, and are found in all tissues. TGF-β is produced by many parenchymal cell types, as well as infiltrating cells such as lymphocytes, monocytes/macrophages, and platelets. Following wounding or inflammation, such cells such are potential sources of TGF-β. In general, TGF-β stimulates the production of various extracellular matrix proteins, inhibits the degradation of these matrix proteins, and promotes tissue fibrosis, all of which contribute to the repair and restoration of the affected tissue. In many diseases, excessive TGF-β contributes to a pathologic excess of tissue fibrosis that can compromise normal organ function.

Examples of TGF-β antagonists include, but are not limited to: monoclonal and polyclonal antibodies directed against one or more isoforms of TGF-β (Dasch et al., U.S. Pat. No. 5,571,714; see, also, WO 97/13844 and WO 00/66631); TGF-β receptors, soluble forms of such receptors (preferably soluble TGF-P type III receptor), or antibodies directed against TGF-β receptors (Segarini et al., U.S. Pat. No. 5,693,607; Lin et al., U.S. Pat. Nos. 6,001,969, 6,010,872, 6,086,867, 6,201,108: WO 98/48024; WO 95/10610: WO 93/09228; WO 92/00330); latency associated peptide (WO 91/08291); large latent TGF-β (WO 94/09812); fetuin (U.S. Pat. No. 5,821,227); decorin and other proteoglycans such as biglycan, fibromodulin, lumican and endoglin (WO 91110727; Ruoslahti et al., U.S. Pat. Nos. 5,654,270, 5,705,609, 5,726,149; Border, U.S. Pat. No. 5,824,655; WO 91/04748; Letarte et al., U.S. Pat. Nos. 5,830,847, 6,015,693: WO 91110727; WO 93/09800; and WO 94110187); somatostatin (WO 98/08529); mannose-6-phosphate or mannose-1-phosphate (Ferguson, U.S. Pat. No. 5,520,926); prolactin (WO 97/40848); insulin-like growth factor II (WO 98117304); IP-10 (WO 97/00691); arg-gly-asp containing peptides (Pfeffer, U.S. Pat. No. 5,958,411; WO 93/10808); extracts of plants, fungi and bacteria (EP-A-813 875; JP 8119984; and Matsunaga et al., U.S. Pat. No. 5,693,610); antisense oligonucleotides (Chung, U.S. Pat. No. 5,683,988; Fakhrai et al., U.S. Pat. No. 5,772,995; Dzau, U.S. Pat. Nos. 5,821,234, 5,869,462; and WO 94/25588): proteins involved in TGF-β signaling, including SMADs and MADs (EP-A-874 046; WO 97/31020; WO 97/38729; WO 98/03663; WO 98/07735; WO 98/07849; WO 98/45467; WO 98/53068; WO 98/55512; WO 98/56913; WO 98/53830; WO 99/50296; Falb, U.S. Pat. No. 5,834,248; Falb et al., U.S. Pat. No. 5,807,708; and Gimeno et al., U.S. Pat. No. 5,948,639), Ski and Sno (Vogel, 1999, Science, 286:665; and Stroschein et al., 1999, Science, 286:771-774); one or more singlestranded oligonucleotide aptamers, or expression plasmids encoding thereof, that are suitable for inhibiting or interfering with the binding of TGF-β to its cognate receptors: and any mutants, fragments or derivatives of the above-identified molecules that retain the ability to inhibit the activity of TGF-β.

In some embodiments, the TGF-β antagonist is a human or humanized monoclonal antibody that blocks TGF-β binding to its receptor (or fragments thereof such as F(ab)2 fragments, Fv fragments, single chain antibodies and other forms or fragments of antibodies that retain the ability to bind to TGF-β, e.g., a monoclonal antibody from hybridoma 1 D 11.16 (ATCC Accession No. HB 9849 described in Dasch et al., U.S. Pat. No. 5,783,185).

### Anti-Fibrotic Agents

In certain embodiments, the profibrotic factor antagonists can be replaced with, or augmented with, a cytokine known to have anti-fibrotic effects, such as IL-12, IL-10, IFN-g or BMP-7 (OP-1). The nucleic acid sequences encoding IFN-y polypeptides may be accessed from public databases, e.g. Genbank, journal publications, etc. While various mammalian IFN-g polypeptides are of interest, for the treatment of human disease, generally the human protein will be used. Human IFN-g coding sequence may be found in Genbank, accession numbers P01579 and CAA00375. The corresponding genomic sequence may be found in Genbank, accession numbers 100219; M37265; and V00536. See, for example. Gray et al. (1982) Nature 295:501 (Genbank X13274); and Rinderknecht et al. (1984) J. Biol. Chern. 259:6790. IFN-g1b (Actimmune®; human interferon) is a single-chain polypeptide of 140 amino acids. It is made recombinantly in E. coli and is unglycosylated. Rinderknecht et al. (1984) J. Biol. Chern. 259:6790-6797. The IFN-g to be used in anti-fibrotic therapy may be any of natural IFN-g, recombinant IFN-g and the derivatives thereof so far as they have a IFN-g activity, particularly human IFN-g activity.

Anti-fibrotic therapy comprises one or more Serum amyloid P (SAP) agonists, one or more C-reative protein (CRP) antagonists, or a combination thereof. SAP agonists are useful in the treatment of various fibrotic disorders. SAP agonists encompass all compounds and compositions that increase or otherwise mimic endogenous SAP signaling, including compounds that increase SAP activity.

Serum amyloid P (SAP) or pentraxin-2, a member of the pentraxin family, is a 27-kDa protein that is produced by the liver, secreted into the blood, and circulates as stable 135-kDa pentamers. SAP reduces neutrophil adhesion to ECM proteins, inhibits the differentiation of monocytes into fibrocytes, decreases profibrotic macrophages, activates the complement pathway, and promotes phagocytosis of cell debris. SAP reduces bleomycin-induced lung fibrosis. Representative agonists of SAP includes human SAP protein or an active fragment thereof, anti-FcyR antibodies, aggregated antibodies, SAP peptidomimetic, FcyR crosslinkers, and aptamers. CRP antagonists encompass all compounds and compositions that decrease, block, or inhibit CRP signaling. Representative antagonists include an anti-sense nucleic acid that targets the expression of CRP, RNA interference molecules, e.g., short interfering RNA, dsRNA, and Locked Nucleic Acids (LNAs).

### Nucleic Acid Molecules, Vectors, Host Cells

Nucleic acid molecules that encode the polypeptides disclosed herein, e.g., the NP-011 protein, are provided.

Nucleic acid molecules disclosed herein can be in the form of RNA or in the form of DNA. DNA includes cDNA, genomic DNA, and synthetic DNA: and can be double-stranded or single-stranded, and if single stranded can be the coding strand/or non-coding (anti-sense) strand. In certain embodiments, the nucleic acid molecule is isolated. In additional embodiments, a nucleic acid molecule is substantially pure. In some embodiments, the nucleic acid is cDNA or is derived from cDNA. In some embodiments, the nucleic acid is recombinantly produced.

A nucleic acid molecule of the present invention can be amplified using cDNA, mRNA, or genomic DNA as a template and appropriate oligonucleotide primers according to standard PCR amplification techniques. The nucleic acid molecules so amplified can be cloned into an appropriate vector and characterized by DNA sequence analysis. Furthermore, oligonucleotides corresponding to all or a portion of a nucleic acid molecule of the present invention can be prepared by standard synthetic techniques, e.g., using an automated DNA synthesizer.

In some embodiments, the nucleic acid molecule comprises a protein coding sequence operably linked to a control sequence that controls the expression of the coding sequence in a host cell or in vitro (see below). In particular embodiments, the coding sequence is a cDNA. The disclosure also relates to vectors containing nucleic acid molecules that comprise a protein (e.g., NP-011) coding sequence operably linked to a control sequence that controls the expression of the coding sequence in a host cell or in vitro.

In some embodiments, the nucleic acid molecule comprises a coding sequence for a protein that is fused in the same reading frame to a heterologous polynucleotide sequence. In some embodiments, the heterologous polynucleotide sequence encodes a leader peptide sequence that facilitates the secretion of the expressed protein from the host cell transformed with the polypeptide (e.g., NP-011) encoding nucleic acid molecule(s). A protein containing a leader sequence is referred to as a preprotein and can have the leader sequence cleaved by the host cell to form the processed form of the protein. Such leader peptide sequences and their use facilitating the secretion of recombinant proteins in host cells is generally known in the art. In additional embodiments, the heterologous polynucleotide sequence encodes additional 5′ amino acid residues that can function for example, to facilitate purification, add or improve protein stability and/or therapeutic properties of the recombinantly expressed protein. In preferred embodiments, NP-011 and/or similar proteins are secreted from the host cells. In certain preferred embodiments, NP-011 and/or similar proteins lack the N-terminal methionine and do not comprise a heterologous sequence. In other preferred embodiments, NP-011 comprises a heterologous sequence that extends the half-life in vivo.

In some embodiments, a nucleic acid sequence encoding the protein or a vector comprising said nucleic acid sequence is constructed by chemical synthesis using an oligonucleotide synthesizer. Such oligonucleotides can be designed based on the amino acid sequence of the desired polypeptide and codon optimization based on the host cell preferences. Standard methods can routinely be applied to synthesize and isolate polynucleotide sequences encoding the polypeptide (e.g., NP-011).

The vector may be a construct, which is capable of delivering, and in some embodiments, expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

Once assembled (by synthesis or another method), the nucleic acid sequences encoding the polypeptide can routinely be operably linked to a control sequence appropriate for expression in a desired host. In some embodiments, the nucleic acid sequence(s) encoding the polypeptide is inserted into one or more expression vectors and operably linked to a control sequence(s) appropriate for expression of the protein in a desired host. In order to obtain high expression levels of a transfected coding sequence in a host, the coding sequence can be operably linked to or associated with transcriptional and translational expression control sequences that are functional in the chosen expression host.

In certain embodiments, recombinant expression vectors are used to amplify and express DNA encoding the polypeptide. Recombinant expression vectors are replicable DNA constructs which have synthetic or cDNA-derived DNA fragments encoding the polypeptide (e.g., NP-011) operably linked to suitable transcriptional or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence which is transcribed into mRNA and translated into protein, and (3) appropriate transcription and translation initiation and termination sequences, as described in detail below. Such regulatory elements can include an operator sequence to control transcription. The ability to replicate in a host, usually conferred by an origin of replication, and a selection gene to facilitate recognition of transformants can additionally be incorporated. DNA regions are operably linked when they are functionally related to each other. For example, DNA for a signal peptide (secretory leader) is operably linked to DNA for a polypeptide if it is expressed as a precursor which participates in the secretion of the polypeptide; a promoter is operably linked to a coding sequence if it controls the transcription of the sequence; or a ribosome binding site is operably linked to a coding sequence if it is positioned so as to permit translation. Structural elements intended for use in yeast expression systems include a leader sequence enabling extracellular secretion of translated protein by a host cell. Alternatively, where a recombinant protein is expressed without a leader or transport sequence, the protein can include an N-terminal methionine residue. This residue can optionally be subsequently cleaved from the expressed recombinant protein to provide a final protein. In certain embodiments, the disclosure provides a composition, e.g., a pharmaceutical composition, comprising a nucleic acid or vector of as described above or elsewhere herein, optionally further comprising one or more carriers, diluents, excipients, or other additives.

Also provided is a host cell transformed with the nucleic acid molecule or cDNA molecules and/or the vectors disclosed herein. The disclosure also provides host cells transformed with the disclosed nucleic acid molecule or molecules operably linked to a control sequence and optionally inserted into a vector.

In additional embodiments, the disclosure provides a method of making the polypeptides provided herein comprising culturing a transformed host cell herein under suitable conditions for producing the the polypeptide. The disclosure optionally provides isolating the polypeptide secreted from the host cell. The disclosure also optionally provides the polypeptide produced using this method and pharmaceutical compositions comprising the the polypeptide and a pharmaceutically acceptable carrier.

The choice of expression control sequence and expression vector will depend upon the choice of host. A wide variety of expression host/vector combinations can be employed (see also below for additional examples). Useful expression vectors for eukaryotic hosts, include, for example, vectors comprising expression control sequences from SV40, bovine papilloma virus, adenovirus and cytomegalovirus. Useful expression vectors for bacterial hosts include known bacterial plasmids, such as plasmids from E. coli, including pCRI, pBR322, pMB9 and their derivatives, and also wider host range plasmids, such as M13 and filamentous single-stranded DNA phages.

A host cell may be a cell or a population of cells harboring or capable of harboring a recombinant nucleic acid. Host cells can be prokaryotic (e.g., E. coli), or eukaryotic. Prokaryotes include gram negative or gram positive organisms, for example E. coli or bacilli. The host cells can be fungal cells including yeast such as Saccharomyces cerevisiae, Pichia pastoris, or Schizosaccharomyces pombe. The host cells also be any of various animal cells, such as insect cells (e.g., Sf-9) or established cell lines of mammalian origin. Examples of suitable mammalian host cell lines include HEK-293, HEK293F, and HEK-293T, the COS-7 lines of monkey kidney cells, described by Gluzman (Cell 23:175 (1981)), and other cell lines including, for example, L cells, C127, 3T3, Chinese hamster ovary (CHO), HeLa and BHK cell lines. Mammalian expression vectors can comprise nontranscribed elements such as an origin of replication, a suitable promoter and enhancer linked to the gene to be expressed, and other 5′ or 3′ flanking nontranscribed sequences, and 5′ or 3′ nontranslated sequences, such as necessary ribosome binding sites, a polyadenylation site, splice donor and acceptor sites, and transcriptional termination sequences. Baculovirus systems for production of heterologous proteins Insect cells are reviewed by Luckow and Summers, BioTechnology 6:47 (1988). Cell-free translation systems could also be employed. In preferred embodiments, the host cell is Pichia pastoris.

### Delivery of Nucleic Acid Molecules, Vectors and Hosts

In some embodiments, the nucleic acid molecules, the vectors, and host cells are provided in pharmaceutical compositions. In some embodiments, a component of pharmaceutical compositions aid delivery of nucleic acid into the intended recipient.

In some embodiments, the DNA constructs are delivered to cells by transfection, i.e., by delivery of “naked” DNA or in a complex with a colloidal dispersion system. A colloidal system includes macromolecule complexes, nanocapsules, microspheres, beads, and lipid-based systems including oil-in-water emulsions, micelles, mixed micelles, and liposomes. The preferred colloidal system of this invention is a lipid-complexed or liposome-formulated DNA. In the former approach, prior to formulation of DNA, e.g., with lipid, a plasmid containing a transgene bearing the desired DNA constructs may first be experimentally optimized for expression (e.g., inclusion of an intron in the 5′ untranslated region and elimination of unnecessary sequences (Felgner, et al., Ann NY Acad Sci 126-139, 1995). Formulation of DNA, e.g. with various lipid or liposome materials, may then be effected using known methods and materials and delivered to the recipient mammal. See, e.g., Canonico et al, Am J Respir Cell Mol Biol 10:24-29, 1994; Tsan et al, Am J Physiol 268; Alton et al., Nat Genet. 5:135-142, 1993 and U.S. Pat. No. 5,679,647 by Carson et al.

The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs, which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

The surface of the targeted delivery system may be modified in a variety of ways. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand. Naked DNA or DNA associated with a delivery vehicle, e.g., liposomes, can be administered to several sites in a subject.

Nucleic acids can be delivered in any desired vector. These include viral or non-viral vectors, including adenovirus vectors, adeno-associated virus vectors, retrovirus vectors, lentivirus vectors, and plasmid vectors. Exemplary types of viruses include HSV (herpes simplex virus), AAV (adeno associated virus), HIV (human immunodeficiency virus), BIV (bovine immunodeficiency virus), and MLV (murine leukemia virus). Nucleic acids can be administered in any desired format that provides sufficiently efficient delivery levels, including in virus particles, in liposomes, in nanoparticles, and complexed to polymers.

The nucleic acids encoding a protein or nucleic acid of interest may be in a plasmid or viral vector, or other vector as is known in the art. Such vectors are well known and any can be selected for a particular application. In one embodiment of the present invention, the gene delivery vehicle comprises a promoter and a demethylase coding sequence. Preferred promoters are tissue-specific promoters and promoters which are activated by cellular proliferation, such as the thymidine kinase and thymidylate synthase promoters. Other preferred promoters include promoters which are activatable by infection with a virus, such as the α- and β-interferon promoters, and promoters which are activatable by a hormone, such as estrogen. Other promoters which can be used include the Moloney virus LTR, the CMV promoter, and the mouse albumin promoter. A promoter may be constitutive or inducible.

In another embodiment, naked polynucleotide molecules are used as gene delivery vehicles, as described in WO 90/11092 and U.S. Pat. No. 5,580,859. Such gene delivery vehicles can be either growth factor DNA or RNA and, in certain embodiments, are linked to killed adenovirus. Curiel et al., Hum. Gene. Ther. 3:147-154, 1992. Other vehicles which can optionally be used include DNA-ligand (Wu et al., J. Biol. Chem. 264:16985-16987, 1989), lipid-DNA combinations (Felgner et al., Proc. Natl. Acad. Sci. USA 84:7413 7417, 1989), liposomes (Wang et al., Proc. Natl. Acad. Sci. 84:7851-7855, 1987) and microprojectiles (Williams et al., Proc. Natl. Acad. Sci. 88:2726-2730, 1991).

A gene delivery vehicle can optionally comprise viral sequences such as a viral origin of replication or packaging signal. These viral sequences can be selected from viruses such as astrovirus, coronavirus, orthomyxovirus, papovavirus, paramyxovirus, parvovirus, picornavirus, poxvirus, retrovirus, togavirus or adenovirus. In a preferred embodiment, the growth factor gene delivery vehicle is a recombinant retroviral vector. Recombinant retroviruses and various uses thereof have been described in numerous references including. for example, Mann et al., Cell 33:153, 1983, Cane and Mulligan, Proc. Nat'l. Acad. Sci. USA 81:6349, 1984, Miller et al., Human Gene Therapy 1:5-14, 1990, U.S. Pat. Nos. 4,405,712, 4,861,719, and 4,980,289, and PCT Application Nos. WO 89/02,468, WO 89/05,349, and WO 90/02,806. Numerous retroviral gene delivery vehicles can be utilized in the present invention, including for example those described in EP 0,415,731; WO 90/07936; WO 94/03622; WO 93/25698; WO 93/25234; U.S. Pat. No. 5,219,740; WO 9311230; WO 9310218; Vile and Hart, Cancer Res. 53:3860-3864, 1993; Vile and Hart, Cancer Res. 53:962-967, 1993; Ram et al., Cancer Res. 53:83-88, 1993; Takamiya et al., J. Neurosci. Res. 33:493-503, 1992; Baba et al., J. Neurosurg. 79:729-735, 1993 (U.S. Pat. No. 4,777,127, GB 2,200,651, EP 0,345,242 and WO91/02805).

Other viral vector systems that can be used to deliver a polynucleotide of the present invention have been derived from herpes virus, e.g., Herpes Simplex Virus (U.S. Pat. No. 5,631,236 by Woo et al., issued May 20, 1997 and WO 00/08191 by Neurovex), vaccinia virus (Ridgeway (1988) Ridgeway, “Mammalian expression vectors,” In: Rodriguez R L, Denhardt D T, ed. Vectors: A survey of molecular cloning vectors and their uses. Stoneham: Butterworth; Baichwal and Sugden (1986) “Vectors for gene transfer derived from animal DNA viruses: Transient and stable expression of transferred genes,” In: Kucherlapati R, ed. Gene transfer. New York: Plenum Press; Coupar et al. (1988) Gene, 68:1-10), and several RNA viruses. Preferred viruses include an alphavirus, a poxivirus, an arena virus, a vaccinia virus, a polio virus, and the like. They offer several attractive features for various mammalian cells (Friedmann (1989) Science, 244:1275-1281; Ridgeway, 1988, supra; Baichwal and Sugden, 1986, supra; Coupar et al., 1988; Horwich et al. (1990) J. Virol., 64:642-650).

### Methods of Producing a Polypeptide

In some embodiments, vectors comprising nucleic acids encoding the polypeptide (e.g., NP-011) may be transfected into host cells. As disclosed herein, the host cells may be cultured in a culture medium to produce the polypeptide. In some embodiments, the polypeptide is secreted into the medium. The polypeptide may be isolated from the medium using any suitable technique.

The polypeptides of the present invention (e.g., NP-011) produced by a transformed host cell can be purified according to any suitable method. Such standard methods include chromatography (e.g., ion exchange, affinity and sizing column chromatography), centrifugation, differential solubility, or by any other standard technique for protein purification. Affinity tags such as hexahistidine, maltose binding domain, influenza coat sequence and glutathione-S-transferase can be attached to the protein to allow easy purification by passage over an appropriate affinity column. The polypeptide can also be physically characterized using such techniques as proteolysis, nuclear magnetic resonance and x-ray crystallography.

For example, supernatants from systems that secrete the polypeptides into culture media can be first concentrated using a commercially available protein concentration filter, for example, an Amicon or Millipore Pellicon ultrafiltration unit. Following the concentration step, the concentrate can be applied to a suitable purification matrix. Alternatively, an anion exchange resin can be employed, for example, a matrix or substrate having pendant diethylaminoethyl (DEAE) groups. The matrices can be acrylamide, agarose, dextran, cellulose or other types commonly employed in protein purification. Alternatively, a cation or anion exchange step can be employed. Suitable cation exchangers include various insoluble matrices comprising sulfopropyl or carboxymethyl groups. Finally, one or more reversed-phase high performance liquid chromatography (RP-HPLC) steps employing hydrophobic RP-HPLC media, e.g., silica gel having pendant methyl or other aliphatic groups, can be employed to further purify the polypeptide.

The polypeptide produced in cell culture can be isolated, for example, by initial extraction from cell pellets, followed by one or more concentration, salting-out, aqueous ion exchange or size exclusion chromatography steps. High performance liquid chromatography (HPLC) can be employed for final purification steps. Cells employed in expression of a recombinant protein can be disrupted by any convenient method, including freeze-thaw cycling, sonication, mechanical disruption, or use of cell lysing agents.

An “isolated” or “purified” protein or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, 20%, 10%, or 5% (by dry weight) of heterologous protein (also referred to herein as a “contaminating protein”). When the protein or biologically active portion thereof is recombinantly produced, it is also preferably substantially free of culture medium, i.e., culture medium represents less than about 20%, 10%, or 5% of the volume of the protein preparation. When the protein is produced by chemical synthesis, it is preferably substantially free of chemical precursors or other chemicals, i.e., it is separated from chemical precursors or other chemicals which are involved in the synthesis of the protein. Accordingly such preparations of the protein have less than about 30%, 20%, 10%, 5% (by dry weight) of chemical precursors or compounds other than the polypeptide of interest.

### Pharmaceutical Composition

In another aspect, the present invention provides pharmaceutically acceptable compositions which comprise a therapeutically-effective amount of the polypeptide disclosed herein formulated together with one or more pharmaceutically acceptable carriers (additives) and/or diluents. The pharmaceutical compositions of the present invention may be specially formulated for storage and use by combining a purified agent of the present invention encompassed by the present invention with a pharmaceutically acceptable vehicle (e.g., a carrier or excipient). Those of skill in the art generally consider pharmaceutically acceptable carriers, excipients, and/or stabilizers to be inactive ingredients of a formulation or pharmaceutical composition.

Suitable pharmaceutically acceptable vehicles include, but are not limited to, nontoxic buffers such as phosphate, citrate, and other organic acids: salts such as sodium chloride; antioxidants including ascorbic acid and methionine; preservatives such as octadecyldimethylbenzyl ammonium chloride, hexamethonium chloride, benzalkonium chloride, benzethonium chloride, phenol, butyl or benzyl alcohol, alkyl parabens, such as methyl or propyl paraben, catechol, resorcinol, cyclohexanol, 3-pentanol, and m-cresol; low molecular weight polypeptides (e.g., less than about 10 amino acid residues); proteins such as serum albumin, gelatin, or immunoglobulins; hydrophilic polymers such as polyvinylpyrrolidone; amino acids such as glycine, glutamine, asparagine, histidine, arginine, or lysine; carbohydrates such as monosaccharides, disaccharides, glucose, mannose, or dextrins; chelating agents such as EDTA; sugars such as sucrose, mannitol, trehalose or sorbitol; salt-forming counter-ions such as sodium; metal complexes such as Zn-protein complexes; and non-ionic surfactants such as TWEEN or polyethylene glycol (PEG). (Remington: The Science and Practice of Pharmacy, 22.sup.nd Edition, 2012, Pharmaceutical Press, London.). Formulations to be used for in vivo administration must be sterile. This is readily accomplished by filtration through sterile filtration membranes.

Examples of pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Pharmaceutically acceptable salts may be part of the pharmaceutical composition comprising the polypeptide of present invention. The salts may be relatively non-toxic, inorganic and organic acid addition to the formulation. The salts may be added to the polypeptide at any stage of the purification (including after purification). Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like (See, for example, Berge et al. (1977) “Pharmaceutical Salts”, J. Pharm. Sci. 66:1-19).

Wetting agents, emulsifiers and lubricants, such as sodium lauryl sulfate and magnesium stearate, as well as release agents, preservatives and antioxidants can also be present in the compositions. Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Pharmaceutical compositions of this invention suitable for parenteral administration may comprise one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In certain embodiments, pharmaceutical formulations include polypeptide complexed with liposomes. Methods to produce liposomes are known to those of skill in the art. For example, some liposomes can be generated by reverse phase evaporation with a lipid composition comprising phosphatidylcholine, cholesterol, and PEG-derivatized phosphatidylethanolamine (PEG-PE). Liposomes can be extruded through filters of defined pore size to yield liposomes with the desired diameter.

In certain embodiments, sustained-release preparations comprising the polypeptide of present invention can be produced. Suitable examples of sustained-release preparations include semi-permeable matrices of solid hydrophobic polymers containing the polypeptide, where the matrices are in the form of shaped articles (e.g., films or microcapsules). Examples of sustained-release matrices include polyesters, hydrogels such as poly(2-hydroxyethyl-methacrylate) or poly(vinyl alcohol), polylactides, copolymers of L-glutamic acid and 7 ethyl-L-glutamate, non-degradable ethylene-vinyl acetate, degradable lactic acid-glycolic acid copolymers such as the LUPRON DEPOT (injectable microspheres composed of lactic acid-glycolic acid copolymer and leuprolide acetate), sucrose acetate isobutyrate, and poly-D-(−)-3-hydroxy butyric acid.

The compositions described herein may be administered by any suitable means, including parenteral, subcutaneous, intraperitoneal, intrapulmonary, and intranasal.

Parenteral infusions include intramuscular, intravenous, intraarterial, intraperitoneal, or subcutaneous administration. In addition, the compositions can be suitably administered by pulse infusion.

In some embodiments, the polypeptides of present invention can be conjugated or modified according to well-known pharmacological methods in the art (e.g., pegylation, glycosylation, oligomerization, etc.) in order to further enhance desirable biological activities, such as increased bioavailability and decreased proteolytic degradation.

In some embodiments, the polypeptide is lyophilized and/or stored in a lyophilized form. In some embodiments, a formulation comprising the polypeptide described herein is lyophilized.

### Kits

A “kit” is any manufacture (e.g. a package or container) comprising at least one composition, e.g. a polypeptide, of the present invention. The kit may also comprise additional diluents. The kit may comprise one or more agents necessary to resuspend the lyophilized composition and/or a filtration unit to remove aggregates. The kit may be promoted, distributed, or sold as a unit for performing the methods of the present invention. The kit may comprise one or more reagents necessary to express a composition useful in the methods of the present invention. Reagents in the kit may be provided in individual containers or as mixtures of two or more reagents in a single container. In addition, instructional materials which describe the use of the compositions within the kit may be included.

### Sequences

The term “sequence identity or homology” refers to the sequence similarity between two polypeptide molecules or between two nucleic acid molecules. When a position in both of the two compared sequences is occupied by the same base or amino acid monomer subunit, e.g., if a position in each of two DNA molecules is occupied by adenine, then the molecules are homologous or sequence identical at that position. The percent of homology or sequence identity between two sequences is a function of the number of matching or homologous identical positions shared by the two sequences divided by the number of positions compared ×100. For example, if 6 of 10, of the positions in two sequences are the same then the two sequences are 60% homologous or have 60% sequence identity. By way of example, the DNA sequences ATTGCC and TATGGC share 50% homology or sequence identity. Generally, a comparison is made when two sequences are aligned to give maximum homology. Unless otherwise specified “loop out regions”, e.g., those arising from, from deletions or insertions in one of the sequences are counted as mismatches. The comparison of sequences and determination of percent homology between two sequences can be accomplished using a mathematical algorithm.

In addition to naturally-occurring allelic variants of a nucleic acid molecule of the present invention that can exist in the population, the skilled artisan will further appreciate that sequence changes can be introduced by mutation thereby leading to changes in the amino acid sequence of the encoded protein, without altering the biological activity of the protein encoded thereby. For example, one can make nucleotide substitutions leading to amino acid substitutions at “non-essential” amino acid residues. A “non-essential” amino acid residue is a residue that can be altered from the wild-type sequence without altering the biological activity, whereas an “essential” amino acid residue is required for biological activity. For example, amino acid residues that are not conserved or only semi-conserved among homologs of various species may be non-essential for activity and thus would be likely targets for alteration. Alternatively, amino acid residues that are conserved among the homologs of various species (e.g., murine and human) may be essential for activity and thus would not be likely targets for alteration.

Accordingly, another aspect of the present invention pertains to nucleic acid molecules encoding a polypeptide of the present invention that contain changes in amino acid residues that are not essential for activity. Such polypeptides differ in amino acid sequence from the naturally-occurring proteins which correspond to the markers of the present invention, yet retain biological activity. In one embodiment, a polypeptide has an amino acid sequence that is at least about 40% identical, 50%, 60%, 70%, 75%, 80%, 83%, 85%, 87.5%, 90%, 91%, 92%, 93%, 94%, 95%, 96%, 97%, 98%, 99% or identical to the amino acid sequence of a polypeptide described herein.

Nucleic acid variants may contain alterations in the coding regions, non-coding regions, or both. In some embodiments, the nucleic acid variants contain alterations that produce silent substitutions, additions, or deletions, but do not alter the properties or activities of the encoded polypeptide. In some embodiments, the nucleic acid variants are produced by silent substitutions due to the degeneracy of the genetic code. Nucleic acid variants can be produced for a variety of reasons, e.g., to optimize codon expression for a particular host (change codons in the human mRNA to those preferred by a bacterial host such as E. coli). Vectors and cells comprising the nucleic acids described herein are also provided.

An isolated nucleic acid molecule encoding a variant protein can be created by introducing one or more nucleotide substitutions, additions or deletions into the nucleotide sequence of nucleic acids of the present invention, such that one or more amino acid residue substitutions, additions, or deletions are introduced into the encoded protein. Mutations can be introduced by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed recombinantly and the activity of the protein can be determined.

Non-conservative substitutions include those in which (a) a residue having an electropositive side chain (e.g., Arg, His, or Lys) is substituted for, or by, an electronegative residue (e.g., Glu or Asp), (b) a hydrophilic residue (e.g., Ser or Thr) is substituted for, or by, a hydrophobic residue (e.g., Ala, Leu, Ile, Phe, or Val), (c) a Cys or Pro is substituted for, or by, any other residue, or (d) a residue having a bulky hydrophobic or aromatic side chain (e.g., Val, His, Ile, or Trp) is substituted for, or by, one having a smaller side chain (e.g., Ala or Ser) or no side chain (e.g., Gly).

Other substitutions can be readily identified. For example, for the amino acid alanine, a substitution can be taken from any one of D-Ala, Gly, beta-Ala, L-Cys and D-Cys. For lysine, a replacement can be any one of D-Lys, Arg. D-Arg, homo-Arg, Met, D-Met, ornithine, or D-ornithine. Generally, substitutions in functionally important regions that can be expected to induce changes in the properties of isolated polypeptides are those in which (a) a polar residue (e.g., Ser or Thr) is substituted for (or by) a hydrophobic residue (e.g., Leu, Ile, Phe, or Ala); (b) a Cys residue is substituted for (or by) any other residue; (c) a residue having an electropositive side chain (e.g., Lys, Arg, or His), is substituted for (or by) a residue having an electronegative side chain (e.g., Glu or Asp); or (d) a residue having a bulky side chain (e.g., Phe) is substituted for (or by) one not having such a side chain (e.g., Gly). The likelihood that one of the foregoing non-conservative substitutions can alter functional properties of the protein is also correlated to the position of the substitution with respect to functionally important regions of the protein: some non-conservative substitutions can accordingly have little or no effect on biological properties.

There is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid and the amino acid sequence encoded by that nucleic acid, as defined by the genetic code.

GENETIC CODE

| | |
|---|---|
| Alanine (Ala, A) | GCA, GCC, GCG, GCT |
| Arginine (Arg, R) | AGA, ACG, CGA, CGC, CGG, CGT |
| Asparagine (Asn, N) | AAC, AAT |
| Aspartic acid (Asp, D) | GAC, GAT |
| Cysteine (Cys, C) | TGC, TGT |
| Glutamic acid (Glu, E) | GAA, GAG |
| Glutamine (Gln, Q) | CAA, CAG |
| Glycine (Gly, G) | GGA, GGC, GGG, GGT |
| Histidine (His, H) | CAC, CAT |
| Isoleucine (Ile, I) | ATA, ATC, ATT |
| Leucine (Leu, L) | CTA, CTC, CTG, CTT, TTA, TTG |
| Lysine (Lys, K) | AAA, AAG |
| Methionine (Met, M) | ATG |
| Phenylalanine (Phe, F) | TTC, TTT |
| Proline (Pro, P) | CCA, CCC, CCG, CCT |
| Serine (Ser, S) | AGC, AGT, TCA, TCC, TCG, TCT |
| Threonine (Thr, T) | ACA, ACC, ACG, ACT |
| Tryptophan (Trp, W) | TGG |
| Tyrosine (Tyr, Y) | TAC, TAT |
| Valine (Val, V) | GTA, GTC, GTG, GTT |
| Termination signal | TAA, TAG, TGA |
| (end) | |

An important and well known feature of the genetic code is its redundancy, whereby, for most of the amino acids used to make proteins, more than one coding nucleotide triplet may be employed (illustrated above). Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In view of the foregoing, the nucleotide sequence of a DNA or RNA coding for a polypeptide of the invention (or any portion thereof) can be used to derive the fusion protein or polypeptide amino acid sequence, using the genetic code to translate the DNA or RNA into an amino acid sequence. Likewise, for a polypeptide amino acid sequence, corresponding nucleotide sequences that can encode the fusion protein or polypeptide can be deduced from the genetic code (which, because of its redundancy, will produce multiple nucleic acid sequences for any given amino acid sequence). Thus, description and/or disclosure herein of a nucleotide sequence which encodes a fusion protein or polypeptide should be considered to also include description and/or disclosure of the amino acid sequence encoded by the nucleotide sequence. Similarly, description and/or disclosure of a fusion protein or polypeptide amino acid sequence herein should be considered to also include description and/or disclosure of all possible nucleotide sequences that can encode the amino acid sequence.

Finally, nucleic acid and amino acid sequence information for nucleic acid and polypeptide molecules useful in the present invention are well known in the art and readily available on publicly available databases, such as the National Center for Biotechnology Information (NCBI). For example, exemplary nucleic acid and amino acid sequences derived from publicly available sequence databases are provided in Table 1 below.

TABLE 1
SEQ ID NO: 1 Human milk fat globule-EGF factor 8 protein (MFGE8),
transcript variant 2, cDNA (NM_001114614.3, CDS from 61 to 1068)

| | |
|---|---|
| 1 | agaaccccgc ggggtctgag cagcccagcg tgcccattcc agcgcccgcg tccccgcagc |
| 61 | atgccgcgcc cccgcctgct ggccgcgctg tgcggcgcgc tgctctgcgc ccccagcctc |
| 121 | ctcgtcgccc tggatatctg ttccaaaaac ccctgccaca acggtggttt atgcgaggag |
| 181 | atttcccaag aagtgcgagg agatgtcttc ccctcgtaca cctgcacgtg ccttaagggc |
| 241 | tacgcgggca accactgtga gacgaaatgt gtcgagccac tgggcctgga gaatgggaac |
| 301 | attgccaact cacagatcgc cgcctcgtct gtgcgtgtga ccttcttggg tttgcagcat |
| 361 | tgggtcccgg agctggcccg cctgaaccgc gcaggcatgg tcaatgcctg gacacccagc |
| 421 | agcaatgacg ataacccctg gatccaggtg aacctgctgc ggaggatgtg ggtaacaggt |
| 481 | gtggtgacgc agggtgccag ccgcttggcc agtcatgagt acctgaaggc cttcaaggtg |
| 541 | gcctacagcc ttaatggaca cgaattcgat ttcatccatg atgttaataa aaaacacaag |
| 601 | gagtttgtgg gtaactggaa caaaaacgcg gtgcatgtca acctgtttga gacccctgtg |
| 661 | gaggctcagt acgtgagatt gtaccccacg agctgccaca cggcctgcac tctgcgcttt |
| 721 | gagctactgg gctgtgagct gaacggatgc gccaatcccc tgggcctgaa gaataacagc |
| 781 | atccctgaca agcagatcac ggcctccagc agctacaaga cctggggctt gcatctcttc |
| 841 | agctggaacc cctcctatgc acggctggac aagcagggca acttcaacgc ctgggttgcg |
| 901 | gggagctacg gtaacgatca gtggctgcag atcttccctg gcaactggga caaccactcc |
| 961 | cacaagaaga acttgtttga gacgcccatc ctggctcgct atgtgcgcat cctgcctgta |
| 1021 | gcctggcaca accgcatcgc cctgcgcctg gagctgctgg gctgttagtg gccacctgcc |
| 1081 | acccccaggt cttcctgctt tccatgggcc cgctgcctct tggcttctca gcccctttaa |
| 1141 | atcaccatag ggctggggac tggggaaggg gagggtgttc agaggcagca ccaccacaca |
| 1201 | gtcacccctc cctccctctt tcccaccctc cacctctcac gggccctgcc ccagccccta |
| 1261 | agccccgtcc cctaaccccc agtcctcact gtcctgtttt cttaggcact gagggatctg |
| 1321 | agtaggtctg ggatggacag gaaagggcaa agtagggcgt gtggtttccc tgcccctgtc |
| 1381 | cggaccgccg atcccaggtg cgtgtgtctc tgtctctcct agcccctctc tcacacatca |
| 1441 | cattcccatg gtggcctcaa gaaaggcccg gaagcgccag gctggagata acagcctctt |
| 1501 | gcccgtcggc cctgcgtcgg ccctggggta ccatgtggcc acaactgctg tggccccctg |
| 1561 | tccccaagac acttcccctt gtctccctgg ttgcctctct tgccccttgt cctgaagccc |
| 1621 | agcgacacag aagggggtgg ggcgggtcta tggggagaaa gggagcgagg tcagaggagg |
| 1681 | gcatgggttg gcagggtggg cgtttggggc cctctatgct ggcttttcac cccagaggac |
| 1741 | acaggcagct tccaaaatat atttatcttc ttcacgggaa |

SEQ ID NO: 2 Human lactadherin isoform B (NP_001108086.1)

| | |
|---|---|
| 1 | mprprllaal cgallcapsl lvaldicskn pchngglcee isqevrgdvf psytctclkg |
| 61 | yagnhcetkc veplglengn iansqiaass vrvtflglqh wvpelarlnr agmvnawtps |
| 121 | snddnpwiqv nllrrmwvtg vvtqgasrla sheylkafkv ayslnghefd fihdvnkkhk |
| 181 | efvgnwnkna vhvnlfetpv eagyvrlypt schtactlrf ellgcelngc anplglknns |
| 241 | ipdkqitass syktwglhlf swnpsyarld kqgnfnawva gsygndqwlq ifpgnwdnhs |
| 301 | hkknlfetpi laryvrilpv awhnrialrl ellgc |

SEQ ID NO: 3 Human milk fat globule-EGF factor 8 protein (MFGE8),
transcript variant 4, cDNA (NM_001310319.2, CDS from 61 to 1092)

| | |
|---|---|
| 1 | agaaccccgc ggggtctgag cagcccagcg tgcccattcc agcgcccgcg tccccgcagc |
| 61 | atgccgcgcc cccgcctgct ggccgcgctg tgcggcgcgc tgctctgcgc ccccagcctc |
| 121 | ctcgtcgccc tggaatgtgt cgagccactg ggcctggaga atgggaacat tgccaactca |
| 181 | cagatcgccg cctcgtctgt gcgtgtgacc ttcttgggtt tgcagcattg ggtcccggag |
| 241 | ctggcccgcc tgaaccgcgc aggcatggtc aatgcctgga cacccagcag caatgacgat |
| 301 | aacccctgga tccaggtgaa cctgctgcgg aggatgtggg taacaggtgt ggtgacgcag |
| 361 | ggtgccagcc gcttggccag tcatgagtac ctgaaggcct tcaaggtggc ctacagcctt |
| 421 | aatggacacg aattcgattt catccatgat gttaataaaa aacacaagga gtttgtgggt |
| 481 | aactggaaca aaaacgcggt gcatgtcaac ctgtttgaga cccctgtgga ggctcagtac |
| 541 | gtgagattgt accccacgag ctgccacacg gcctgcactc tgcgctttga gctactgggc |
| 601 | tgtgagctga acggatgcgc caatcccctg ggcctgaaga ataacagcat ccctgacaag |
| 661 | cagatcacgg cctccagcag ctacaagacc tggggcttgc atctcttcag ctggaacccc |
| 721 | tcctatgcac ggctggacaa gcagggcaac ttcaacgcct gggttgcggg gagctacggt |
| 781 | aacgatcagt ggctgcaggt ggacctgggc tcctcgaagg aggtgacagg catcatcacc |
| 841 | cagggggccc gtaactttgg ctctgtccag tttgtggcat cctacaaggt tgcctacagt |
| 901 | aatgacagtg cgaactggac tgagtaccag gaccccagga ctggcagcag taagatcttc |
| 961 | cctggcaact gggacaacca ctcccacaag aagaacttgt ttgagacgcc catcctggct |
| 1021 | cgctatgtgc gcatcctgcc tgtagcctgg cacaaccgca tcgccctgcg cctggagctg |
| 1081 | ctgggctgtt agtggccacc tgccaccccc aggtcttcct gctttccatg ggcccgctgc |
| 1141 | ctcttggctt ctcagcccct ttaaatcacc atagggctgg ggactgggga aggggagggt |
| 1201 | gttcagaggc agcaccacca cacagtcacc cctccctccc tctttcccac cctccacctc |
| 1261 | tcacgggccc tgccccagcc cctaagcccc gtcccctaac ccccagtcct cactgtcctg |
| 1321 | ttttcttagg cactgaggga tctgagtagg tctgggatgg acaggaaagg gcaaagtagg |
| 1381 | gcgtgtggtt tccctgcccc tgtccggacc gccgatccca ggtgcgtgtg tctctgtctc |
| 1441 | tcctagcccc tctctcacac atcacattcc catggtggcc tcaagaaagg cccggaagcg |
| 1501 | ccaggctgga gataacagcc tcttgcccgt cggccctgcg tcggccctgg ggtaccatgt |
| 1561 | ggccacaact gctgtggccc cctgtcccca agacacttcc ccttgtctcc ctggttgcct |
| 1621 | ctcttgcccc ttgtcctgaa gcccagcgac acagaagggg gtggggcggg tctatgggga |
| 1681 | gaaagggagc gaggtcagag gagggcatgg gttggcaggg tgggcgtttg gggccctcta |
| 1741 | tgctggcttt tcaccccaga ggacacaggc agcttccaaa atatatttat cttcttcacg |
| 1801 | ggaa |

SEQ ID NO: 4 Human lactadherin isoform D (NP_001297248.1)

| | |
|---|---|
| 1 | mprprllaal cgallcapsl lvalecvepl glengnians qiaassvrvt flglqhwvpe |
| 61 | larlnragmv nawtpssndd npwiqvnllr rmwvtgvvtq gasrlashey lkafkvaysl |
| 121 | nghefdfihd vnkkhkefvg nwnknavhvn lfetpveaqy vrlyptscht actlrfellg |
| 181 | celngcanpl glknnsipdk qitasssykt wglhlfswnp syarldkqgn fnawvagsyg |
| 241 | ndqwlqvdlg sskevtgiit qgarnfgsvq fvasykvays ndsanwteyq dprtgsskif |
| 301 | pgnwdnhshk knlfetpila ryvrilpvaw hnrialrlel lgc |

SEQ ID NO: 5 Human milk fat globule-EGF factor 8 protein (MFGE8),
transcript variant 3, cDNA (NM_001310320.2, CDS from 138 to 186)

| | |
|---|---|
| 1 | agaaccccgc ggggtctgag cagcccagcg tgcccattcc agcgcccgcg tccccgcagc |
| 61 | atgccgcgcc cccgcctgct ggccgcgctg tgcggcgcgc tgctctgcgc ccccagcctc |
| 121 | ctcgtcgccc tggggtgatg tggccttttc cagaaggagg aaacaccata cctatcttac |
| 181 | acacagatat ctgttccaaa aacccctgcc acaacggtgg tttatgcgag gagatttccc |
| 241 | aagaagtgcg aggagatgtc ttcccctcgt acacctgcac gtgccttaag ggctacgcgg |
| 301 | gcaaccactg tgagacgaaa tgtgtcgagc cactgggcct ggagaatggg aacattgcca |
| 361 | actcacagat cgccgcctcg tctgtgcgtg tgaccttctt gggtttgcag cattgggtcc |
| 421 | cggagctggc ccgcctgaac cgcgcaggca tggtcaatgc ctggacaccc agcagcaatg |
| 481 | acgataaccc ctggatccag gtgaacctgc tgcggaggat gtgggtaaca ggtgtggtga |
| 541 | cgcagggtgc cagccgcttg gccagtcatg agtacctgaa ggccttcaag gtggcctaca |
| 601 | gccttaatgg acacgaattc gatttcatcc atgatgttaa taaaaaacac aaggagtttg |
| 661 | tgggtaactg gaacaaaaac gcggtgcatg tcaacctgtt tgagacccct gtggaggctc |
| 721 | agtacgtgag attgtacccc acgagctgcc acacggcctg cactctgcgc tttgagctac |
| 781 | tgggctgtga gctgaacgga tgcgccaatc ccctgggcct gaagaataac agcatccctg |
| 841 | acaagcagat cacggcctcc agcagctaca agacctgggg cttgcatctc ttcagctgga |
| 901 | acccctccta tgcacggctg gacaagcagg gcaacttcaa cgcctgggtt gcggggagct |
| 961 | acggtaacga tcagtggctg caggtggacc tgggctcctc gaaggaggtg acaggcatca |
| 1021 | tcacccaggg ggcccgtaac tttggctctg tccagtttgt ggcatcctac aaggttgcct |
| 1081 | acagtaatga cagtgcgaac tggactgagt accaggaccc caggactggc agcagtaaga |
| 1141 | tcttccctgg caactgggac aaccactccc acaagaagaa cttgtttgag acgcccatcc |
| 1201 | tggctcgcta tgtgcgcatc ctgcctgtag cctggcacaa ccgcatcgcc ctgcgcctgg |
| 1261 | agctgctggg ctgttagtgg ccacctgcca cccccaggtc ttcctgcttt ccatgggccc |
| 1321 | gctgcctctt ggcttctcag cccctttaaa tcaccatagg gctggggact ggggaagggg |
| 1381 | agggtgttca gaggcagcac caccacacag tcacccctcc ctccctcttt cccaccctcc |
| 1441 | acctctcacg ggccctgccc cagcccctaa gccccgtccc ctaaccccca gtcctcactg |
| 1501 | tcctgttttc ttaggcactg agggatctga gtaggtctgg gatggacagg aaagggcaaa |
| 1561 | gtagggcgtg tggtttccct gcccctgtcc ggaccgccga tcccaggtgc gtgtgtctct |
| 1621 | gtctctccta gcccctctct cacacatcac attcccatgg tggcctcaag aaaggcccgg |
| 1681 | aagcgccagg ctggagataa cagcctcttg cccgtcggcc ctgcgtcggc cctggggtac |
| 1741 | catgtggcca caactgctgt ggccccctgt ccccaagaca cttccccttg tctccctggt |
| 1801 | tgcctctctt gccccttgtc ctgaagccca gcgacacaga agggggtggg gcgggtctat |
| 1861 | ggggagaaag ggagcgaggt cagaggaggg catgggttgg cagggtgggc gtttggggcc |
| 1921 | ctctatgctg gcttttcacc ccagaggaca caggcagctt ccaaaatata tttatcttct |
| 1981 | tcacgggaa |

SEQ ID NO: 6 Human lactadherin isoform C (NP_001297249.1)

| | |
|---|---|
| 1 | mwpfpeggnt ipilhtdics knpchngglc eeisqevrgd vfpsytctcl kgyagnhcet |
| 61 | kcveplglen gniansqiaa ssvrvtflgl qhwypelarl nragmvnawt pssnddnpwi |
| 121 | qvnllrrmwv tgvvtqgasr lasheylkaf kvayslnghe fdfihdvnkk hkefvgnwnk |
| 181 | navhvnlfet pveagyvrly ptschtactl rfellgceln gcanplglkn nsipdkqita |
| 241 | sssyktwglh lfswnpsyar ldkqgnfnaw vagsygndqw lqvdlgsske vtgiitqgar |
| 301 | nfgsvqfvas ykvaysndsa nwteyqdprt gsskifpgnw dnhshkknlf etpilaryvr |
| 361 | ilpvawhnri alrlellgc |

SEQ ID NO: 7 Human milk-fat globule-EGF factor 8 protein (MFGE8),
transcript variant 5, cDNA (NM_001310321.2, CDS from 1005 to 1832)

| | |
|---|---|
| 1 | acctccactg ttgacaaact tagacaaagc cccggggacc gggctgggca gaggggcggc |
| 61 | ttcttccgct gcgccctggc gggacagggg gatgcggccc tgctgtctct gcgctggggc |
| 121 | ttttgggctg ggactcggga catcgggtga cagccctgcc gcccccaggg atgcggctta |
| 181 | cagataatga caaaggaatc cgctgtgtcg ggcctctctt ttccctggtg aaaaatgagg |
| 241 | ccagggaact gcgtttgact ttcgaacccc ttccacctgg gagattctag gactctagta |
| 301 | tggataagtc ttgtctggat aactttgtcc tggccatctc cctgccaact ccagttggct |
| 361 | ggacagttca ttggattttt gcgctcccaa ttgtccgtgc ctggtcacat aagggaaggg |
| 421 | ccggggagtc ggtgcaatgg acgcaggccg taagtggggc ccgggagggg acccagaggc |
| 481 | ttcgaggagc ttggaagagg gctgcctgct gatgggagtc tcctgactcc ctccctcccg |
| 541 | cggccttggc cggctgctgt atcttccccg gtcctcctcc gcctcccagg aggcctccgg |
| 601 | aggccagctg ggccccttgc aggctggact tgcggatgcc ccgtgccatt caccgtggag |
| 661 | cgctgggagg gagtcagggc caggactctt taggtggccc ctccatcatt ttctcataga |
| 721 | aatgggattg actgaagcaa gatatctgtt ccaaaaaccc ctgccacaac ggtggtttat |
| 781 | gcgaggagat ttcccaagaa gtgcgaggag atgtcttccc ctcgtacacc tgcacgtgcc |
| 841 | ttaagggcta cgcgggcaac cactgtgaga cgaaatgtgt cgagccactg ggcctggaga |
| 901 | atgggaacat tgccaactca cagatcgccg cctcgtctgt gcgtgtgacc ttcttgggtt |
| 961 | tgcagcattg ggtcccggag ctggcccgcc tgaaccgcgc aggcatggtc aatgcctgga |
| 1021 | cacccagcag caatgacgat aacccctgga tccaggtgaa cctgctgcgg aggatgtggg |
| 1081 | taacaggtgt ggtgacgcag ggtgccagcc gcttggccag tcatgagtac ctgaaggcct |
| 1141 | tcaaggtggc ctacagcctt aatggacacg aattcgattt catccatgat gttaataaaa |
| 1201 | aacacaagga gtttgtgggt aactggaaca aaaacgcggt gcatgtcaac ctgtttgaga |
| 1261 | cccctgtgga ggctcagtac gtgagattgt accccacgag ctgccacacg gcctgcactc |
| 1321 | tgcgctttga gctactgggc tgtgagctga acggatgcgc caatcccctg ggcctgaaga |
| 1381 | ataacagcat ccctgacaag cagatcacgg cctccagcag ctacaagacc tggggcttgc |
| 1441 | atctcttcag ctggaacccc tcctatgcac ggctggacaa gcagggcaac ttcaacgcct |
| 1501 | gggttgcggg gagctacggt aacgatcagt ggctgcaggt ggacctgggc tcctcgaagg |
| 1561 | aggtgacagg catcatcacc cagggggccc gtaactttgg ctctgtccag tttgtggcat |
| 1621 | cctacaaggt tgcctacagt aatgacagtg cgaactggac tgagtaccag gaccccagga |
| 1681 | ctggcagcag taagatcttc cctggcaact gggacaacca ctcccacaag aagaacttgt |
| 1741 | ttgagacgcc catcctggct cgctatgtgc gcatcctgcc tgtagcctgg cacaaccgca |
| 1801 | tcgccctgcg cctggagctg ctgggctgtt agtggccacc tgccaccccc aggtcttcct |
| 1861 | gctttccatg ggcccgctgc ctcttggctt ctcagcccct ttaaatcacc atagggctgg |
| 1921 | ggactgggga aggggagggt gttcagaggc agcaccacca cacagtcacc cctccctccc |
| 1981 | tctttcccac cctccacctc tcacgggccc tgccccagcc cctaagcccc gtcccctaac |
| 2041 | ccccagtcct cactgtcctg ttttcttagg cactgaggga tctgagtagg tctgggatgg |
| 2101 | acaggaaagg gcaaagtagg gcgtgtggtt tccctgcccc tgtccggacc gccgatccca |
| 2161 | ggtgcgtgtg tctctgtctc tcctagcccc tctctcacac atcacattcc catggtggcc |
| 2221 | tcaagaaagg cccggaagcg ccaggctgga gataacagcc tcttgcccgt cggccctgcg |
| 2281 | tcggccctgg ggtaccatgt ggccacaact gctgtggccc cctgtcccca agacacttcc |
| 2341 | ccttgtctcc ctggttgcct ctcttgcccc ttgtcctgaa gcccagcgac acagaagggg |
| 2401 | gtggggcggg tctatgggga gaaagggagc gaggtcagag gagggcatgg gttggcaggg |
| 2461 | tgggcgtttg gggccctcta tgctggcttt tcaccccaga ggacacaggc agcttccaaa |
| 2521 | atatatttat cttcttcacg ggaa |

SEQ ID NO: 8 Human lactadherin isoform E (NP_001297250.1)

| | |
|---|---|
| 1 | mvnawtpssn ddnpwiqvnl lrrmwvtgvv tqgasrlash eylkafkvay slnghefdfi |
| 61 | hdvnkkhkef vgnwnknavh vnlfetpvea qyvrlyptsc htactlrfel lgcelngcan |
| 121 | plglknnsip dkqitasssy ktwglhlfsw npsyarldkq gnfnawvags ygndqwlqvd |
| 181 | lgsskevtgi itqgarnfgs vqfvasykva ysndsanwte yqdprtgssk ifpgnwdnhs |
| 241 | hkknifetpi laryvrilpv awhnrialrl ellgc |

SEQ ID NO: 9 Human milk fat globule-EGF factor 8 protein (MFGE8),
transcript variant 1, cDNA (NM_005928.4, CDS from 61 to 1224)

| | |
|---|---|
| 1 | agaaccccgc ggggtctgag cagcccagcg tgcccattcc agcgcccgcg tccccgcagc |
| 61 | atgccgcgcc cccgcctgct ggccgcgctg tgcggcgcgc tgctctgcgc ccccagcctc |
| 121 | ctcgtcgccc tggatatctg ttccaaaaac ccctgccaca acggtggttt atgcgaggag |
| 181 | atttcccaag aagtgcgagg agatgtcttc ccctcgtaca cctgcacgtg ccttaagggc |
| 241 | tacgcgggca accactgtga gacgaaatgt gtcgagccac tgggcctgga gaatgggaac |
| 301 | attgccaact cacagatcgc cgcctcgtct gtgcgtgtga ccttcttggg tttgcagcat |
| 361 | tgggtcccgg agctggcccg cctgaaccgc gcaggcatgg tcaatgcctg gacacccagc |
| 421 | agcaatgacg ataacccctg gatccaggtg aacctgctgc ggaggatgtg ggtaacaggt |
| 481 | gtggtgacgc agggtgccag ccgcttggcc agtcatgagt acctgaaggc cttcaaggtg |
| 541 | gcctacagcc ttaatggaca cgaattcgat ttcatccatg atgttaataa aaaacacaag |
| 601 | gagtttgtgg gtaactggaa caaaaacgcg gtgcatgtca acctgtttga gacccctgtg |
| 661 | gaggctcagt acgtgagatt gtaccccacg agctgccaca cggcctgcac tctgcgcttt |
| 721 | gagctactgg gctgtgagct gaacggatgc gccaatcccc tgggcctgaa gaataacagc |
| 781 | atccctgaca agcagatcac ggcctccagc agctacaaga cctggggctt gcatctcttc |
| 841 | agctggaacc cctcctatgc acggctggac aagcagggca acttcaacgc ctgggttgcg |
| 901 | gggagctacg gtaacgatca gtggctgcag gtggacctgg gctcctcgaa ggaggtgaca |
| 961 | ggcatcatca cccagggggc ccgtaacttt ggctctgtcc agtttgtggc atcctacaag |
| 1021 | gttgcctaca gtaatgacag tgcgaactgg actgagtacc aggaccccag gactggcagc |
| 1081 | agtaagatct tccctggcaa ctgggacaac cactcccaca agaagaactt gtttgagacg |
| 1141 | cccatcctgg ctcgctatgt gcgcatcctg cctgtagcct ggcacaaccg catcgccctg |
| 1201 | cgcctggagc tgctgggctg ttagtggcca cctgccaccc ccaggtcttc ctgctttcca |
| 1261 | tgggcccgct gcctcttggc ttctcagccc ctttaaatca ccatagggct ggggactggg |
| 1321 | gaaggggagg gtgttcagag gcagcaccac cacacagtca cccctccctc cctctttccc |
| 1381 | accctccacc tctcacgggc cctgccccag cccctaagcc ccgtccccta acccccagtc |
| 1441 | ctcactgtcc tgttttctta ggcactgagg gatctgagta ggtctgggat ggacaggaaa |
| 1501 | gggcaaagta gggcgtgtgg tttccctgcc cctgtccgga ccgccgatcc caggtgcgtg |
| 1561 | tgtctctgtc tctcctagcc cctctctcac acatcacatt cccatggtgg cctcaagaaa |
| 1621 | ggcccggaag cgccaggctg gagataacag cctcttgccc gtcggccctg cgtcggccct |
| 1681 | ggggtaccat gtggccacaa ctgctgtggc cccctgtccc caagacactt ccccttgtct |
| 1741 | ccctggttgc ctctcttgcc ccttgtcctg aagcccagcg acacagaagg gggtggggcg |
| 1801 | ggtctatggg gagaaaggga gcgaggtcag aggagggcat gggttggcag ggtgggcgtt |
| 1861 | tggggccctc tatgctggct tttcacccca gaggacacag gcagcttcca aaatatattt |
| 1921 | atcttcttca cgggaa |

SEQ ID NO: 10 Human lactadherin isoform A (NP_005919.2)

| | |
|---|---|
| 1 | mprprllaal cgallcapsl lvaldicskn pchngglcee isqevrgdvf psytctclkg |
| 61 | yagnhcetkc veplglengn iansqiaass vrvtflglqh wypelarlnr agmvnawtps |
| 121 | snddnpwiqv nllrrmwvtg vvtqgasrla sheylkafkv ayslnghefd fihdvnkkhk |
| 181 | efvgnwnkna vhvnlfetpv eagyvrlypt schtactlrf ellgcelngc anplglknns |
| 241 | ipdkqitass syktwglhlf swnpsyarld kqgnfnawva gsygndqwlq vdlgsskevt |
| 301 | giitqgarnf gsvqfvasyk vaysndsanw teyqdprtgs skifpgnwdn hshkknlfet |
| 361 | pilaryvril pvawhnrial rlellgc |

*The amino acid sequence (amino acids 24-225) that corresponds to
NP-011(a) is underlined.
Amino acids 1-23 corresponds to the signal peptide.
SEQ ID NO: 11 Nucleic acid encoding human NP-011

| | |
|---|---|
| 1 | agaaccccgc ggggtctgag cagcccagcg tgcccattcc agcgcccgcg tccccgcagc |
| 61 | atgccgcgcc cccgcctgct ggccgcgctg tgcggcgcgc tgctctgcgc ccccagcctc |
| 121 | ctcgtcgccc tggatatctg ttccaaaaac ccctgccaca acggtggttt atgcgaggag |
| 181 | atttcccaag aagtgcgagg agatgtcttc ccctcgtaca cctgcacgtg ccttaagggc |
| 241 | tacgcgggca accactgtga gacgaaatgt gtcgagccac tgggcatgga gaatgggaac |
| 301 | attgccaact cacagatcgc cgcctcgtct gtgcgtgtga ccttcttggg tttgcagcat |
| 361 | tgggtcccgg agctggcccg cctgaaccgc gcaggcatgg tcaatgcctg gacacccagc |
| 421 | agcaatgacg ataacccctg gatccaggtg aacctgctgc ggaggatgtg ggtaacaggt |
| 481 | gtggtgacgc agggtgccag ccgcttggcc agtcatgagt acctgaaggc cttcaaggtg |
| 541 | gcctacagcc ttaatggaca cgaattcgat ttcatccatg atgttaataa aaaacacaag |
| 601 | gagtttgtgg gtaactggaa caaaaacgcg gtgcatgtca acctgtttga gacccctgtg |
| 661 | gaggctcagt acgtgagatt gtaccccacg agctgccaca cggcctgcac tctgcgcttt |
| 721 | gagctactgg gctgtgagct gaacggatgc gccaatcccc tgggcctgaa gaataacagc |
| 781 | atccctgaca agcagatcac ggcctccagc agctacaaga cctggggctt gcatctcttc |
| 841 | agctggaacc cctcctatgc acggctggac aagcagggca acttcaacgc ctgggttgcg |
| 901 | gggagctacg gtaacgatca gtggctgcag gtggacctgg gctcctcgaa ggaggtgaca |
| 961 | ggcatcatca cccagggggc ccgtaacttt ggctctgtcc agtttgtggc atcctacaag |
| 1021 | gttgcctaca gtaatgacag tgcgaactgg actgagtacc aggaccccag gactggcagc |
| 1081 | agtaagatct tccctggcaa ctgggacaac cactcccaca agaagaactt gtttgagacg |
| 1141 | cccatcctgg ctcgctatgt gcgcatcctg cctgtagcct ggcacaaccg catcgccctg |
| 1201 | cgcctggagc tgctgggctg ttagtggcca cctgccaccc ccaggtcttc ctgctttcca |
| 1261 | tgggcccgct gcctcttggc ttctcagccc ctttaaatca ccatagggct ggggactggg |
| 1321 | gaaggggagg gtgttcagag gcagcaccac cacacagtca cccctccctc cctctttccc |
| 1381 | accctccacc tctcacgggc cctgccccag cccctaagcc ccgtccccta acccccagtc |
| 1441 | ctcactgtcc tgttttctta ggcactgagg gatctgagta ggtctgggat ggacaggaaa |
| 1501 | gggcaaagta gggcgtgtgg tttccctgcc cctgtccgga ccgccgatcc caggtgcgtg |
| 1561 | tgtctctgtc tctcctagcc cctctctcac acatcacatt cccatggtgg cctcaagaaa |
| 1621 | ggcccggaag cgccaggctg gagataacag cctcttgccc gtcggccctg cgtcggccct |
| 1681 | ggggtaccat gtggccacaa ctgctgtggc cccctgtccc caagacactt ccccttgtct |
| 1741 | ccctggttgc ctctcttgcc ccttgtcctg aagcccagcg acacagaagg gggtggggcg |
| 1801 | ggtctatggg gagaaaggga gcgaggtcag aggagggcat gggttggcag ggtgggcgtt |
| 1861 | tggggccctc tatgctggct tttcacccca gaggacacag gcagcttcca aaatatattt |
| 1921 | atcttcttca cgggaa |

SEQ ID NO: 12 Human NP-011

| | |
|---|---|
| 1 | mprprllaa1 cgallcapsl lvaldicskn pchngglcee isqevrgdvf psytctclkg |
| 61 | yagnhcetkc veplgmengn iansqiaass vrvtflglqh wypelarlnr agmvnawtps |
| 121 | snddnpwiqv nllrrmwvtg vvtqgasrla sheylkafkv ayslnghefd fihdvnkkhk |
| 181 | efvgnwnkna vhvnlfetpv eagyvrlypt schtactlrf ellgcelngc anplglknns |
| 241 | ipdkqitass syktwglhlf swnpsyarld kqgnfnawva gsygndqwlq vdlgsskevt |
| 301 | giitqgarnf gsvqfvasyk vaysndsanw teyqdprtgs skifpgnwdn hshkknlfet |
| 361 | pilaryvrll pvawhnrlal rlellgc |

*The amino acid sequence (amino acids 24-225) that corresponds to
NP-011 is underlined.
Amino acids 1-23 corresponds to the signal peptide.
SEQ ID NO: 13 Rat milk fat globule-EGF factor 8 protein (Mfge8),
transcript variant 1, cDNA (NM_001040186.2, CDS from 82 to 1533)

| | |
|---|---|
| 1 | attcccctgt gagaggagcg gacgcaggaa ctctccggtc ccagcatcgg agcttgtgga |
| 61 | ccatttcccg cgtcccgcag catgcagttc tcccgtgtgc tggccgcgct gtgcggtgtg |
| 121 | ctgctctgcg cctccggcct cttcgctgcg tccggtgact tctgtgactc cagcctgtgc |
| 181 | ctgaatggtg ggacctgctt gatgggccaa gacaatgaca tctactgcct ctgccctgaa |
| 241 | ggcttcacag gccttgtgtg caacgagact gagaaaggac cgtgttcccc aaacccttgc |
| 301 | ttccacgatg ccaaatgcct ggtgactgag gacacacagc gaggggacat cttcactgag |
| 361 | tacatctgcc agtgccctgt gggctactcg ggcatccact gtgaactcga gaccacctcc |
| 421 | tacctggatg gagagtacct gtccagccca gccgtcccta ccacagccgt ccccaccaca |
| 481 | gccatcccca ccacagccgt ccccaccaca gccgtcccca ccacagccgt ccccaccccg |
| 541 | gcccccaacc ccgatctttc caaccaccta gcctcccgct gttccacaaa gctgggcttg |
| 601 | gaagggggcg ccattgccga ttcacagatt tctgcctcgt ctgtgtatat gggcttcatg |
| 661 | ggcttgcagc gctggggccc ggagctggct cgcctgtatc gcacagggat tgtcaatgca |
| 721 | tggacagcca gcagctatga tagcaagccc tggatccagg tggactttct gcggaagatg |
| 781 | cgggtatcag gtgtgatgac acagggtgcc agccgtgccg ggagggcgga atacctgaag |
| 841 | accttcaagg tggcttacag cctcgatgga cgcaggttcg agttcatcca ggatgaaagc |
| 901 | ggaaccggag acaaggagtt tatgggtaac caggacaaca acagcctgaa gattaacatg |
| 961 | ttcaacccca ctctggaggc acagtacata aggctgtacc ctgtctcgtg ccaccgcggc |
| 1021 | tgcaccctcc gcttcgagct cctgggctgc gagttgcatg gatgctctga gcccctgggc |
| 1081 | ctgaagaata acacgattcc tgacagccaa ataacagcct ccagcagcta caagacgtgg |
| 1141 | aacctgcgtg cctttggctg gtacccccac ttggggcggc tggacaatca gggcaagatc |
| 1201 | aatgcctgga cagctcagag caacagtgcc aaggaatggc tgcaggttga cctgggcact |
| 1261 | cagaaaaaag tgacaggaat tatcacccag ggggcccgtg actttggcca catccagtat |
| 1321 | gtggcatcct ataaggtagc ccacagtgat gatggtgtgc agtggaccgt atatgaggaa |
| 1381 | caaggaacca gcaaggtctt ccagggcaac ttggacaaca actcccacaa gaagaacatc |
| 1441 | tttgagaaac ctttcatggc tcgctatgtg cgtgtccttc cactgtcctg gcataaccgt |
| 1501 | atcaccctgc gcctggagct gctgggctgt tagtgcccag tccttccagc ccaagtgacg |
| 1561 | aggacggcca gaggctgagg ggcctcctgg ccctgcctcc caggccctgc tgccttctgt |
| 1621 | ggctgacacc ttctcaatcc tccctcttga ttgcactggg actacaggca ggaagggcaa |
| 1681 | gggggtttca gagttgcccc tcacccttcc cctcaccctg cagcccccac aggcctcctg |
| 1741 | ctagcccctt ctctcaggca ttctggggga gttggacagg tctgagatga atagagaaga |
| 1801 | agagtgaagt tggggtatgt gggctgctgt accaaccacc ccaagtccta aactttctcc |
| 1861 | aggggttgac tcaagactaa agggaacctc tggttgccca cccgtctctg cacaccgcac |
| 1921 | atccctccat gttccattcc tggaaggaga ggcccacgtc cgcttgctgc cccttgggtc |
| 1981 | accagatcct gcctcttatc tcctgagacc cctcttgacc ctcgctctgg agcctcggtt |
| 2041 | gacaagagga ctgtcgggtc tggagagata gatgggctct gggtggttgg cgagctggct |
| 2101 | atgggacctc tgctggcttg ctacccaagc taacaagcag attccaaaat acatttgtgc |
| 2161 | tctccactgg aaaaaaaaaa aaaaaaaa |

SEQ ID NO: 14 Rat lactadherin isoform 1 (NP_001035276.1)

| | |
|---|---|
| 1 | mqfsrvlaal cgvllcasgl faasgdfcds slclnggtcl mgqdndiycl cpegftglvc |
| 61 | netekgpcsp npcfhdakcl vtedtqrgdi fteyicqcpv gysgihcele ttsyldgey1 |
| 121 | sspavpttav pttaipttav pttavpttav ptpapnpdls nhlasrcstk lgleggaiad |
| 181 | sqisassvym gfmglqrwgp elarlyrtgi vnawtassyd skpwlqvdfl rkmrvsgvmt |
| 241 | qgasragrae ylktfkvays ldgrrfefiq desgtgdkef mgnqdnnslk inmfnptlea |
| 301 | qyirlypvsc hrgctlrfel lgcelhgcse plglknntip dsqitasssy ktwnlrafgw |
| 361 | yphlgrldnq gkinawtaqs nsakewlqvd lgtqkkvtgi itqgardfgh iqyvasykva |
| 421 | hsddgvqwtv yeeqgtskvf qgnldnnshk knifekpfma ryvrvlplsw hnritlrlel |
| 481 | lgc |

SEQ ID NO: 15 Rat milk fat globule-EGF factor 8 protein (Mfge8),
transcript variant 2, cDNA (NM_012811.3, CDS from 82 to 1365)

| | |
|---|---|
| 1 | attcccctgt gagaggagcg gacgcaggaa ctctccggtc ccagcatcgg agcttgtgga |
| 61 | ccatttcccg cgtcccgcag catgcagttc tcccgtgtgc tggccgcgct gtgcggtgtg |
| 121 | ctgctctgcg cctccggcct cttcgctgcg tccggtgact tctgtgactc cagcctgtgc |
| 181 | ctgaatggtg ggacctgctt gatgggccaa gacaatgaca tctactgcct ctgccctgaa |
| 241 | ggcttcacag gccttgtgtg caacgagact gagaaaggac cgtgttcccc aaacccttgc |
| 301 | ttccacgatg ccaaatgcct ggtgactgag gacacacagc gaggggacat cttcactgag |
| 361 | tacatctgcc agtgccctgt gggctactcg ggcatccact gtgaactcgg ctgttccaca |
| 421 | aagctgggct tggaaggggg cgccattgcc gattcacaga tttctgcctc gtctgtgtat |
| 481 | atgggcttca tgggcttgca gcgctggggc ccggagctgg ctcgcctgta tcgcacaggg |
| 541 | attgtcaatg catggacagc cagcagctat gatagcaagc cctggatcca ggtggacttt |
| 601 | ctgcggaaga tgcgggtatc aggtgtgatg acacagggtg ccagccgtgc cgggagggcg |
| 661 | gaatacctga agaccttcaa ggtggcttac agcctcgatg gacgcaggtt cgagttcatc |
| 721 | caggatgaaa gcggaaccgg agacaaggag tttatgggta accaggacaa caacagcctg |
| 781 | aagattaaca tgttcaaccc cactctggag gcacagtaca taaggctgta ccctgtctcg |
| 841 | tgccaccgcg gctgcaccct ccgcttcgag ctcctgggct gcgagttgca tggatgctct |
| 901 | gagcccctgg gcctgaagaa taacacgatt cctgacagcc aaataacagc ctccagcagc |
| 961 | tacaagacgt ggaacctgcg tgcctttggc tggtaccccc acttggggcg gctggacaat |
| 1021 | cagggcaaga tcaatgcctg gacagctcag agcaacagtg ccaaggaatg gctgcaggtt |
| 1081 | gacctgggca ctcagaaaaa agtgacagga attatcaccc agggggcccg tgactttggc |
| 1141 | cacatccagt atgtggcatc ctataaggta gcccacagtg atgatggtgt gcagtggacc |
| 1201 | gtatatgagg aacaaggaac cagcaaggtc ttccagggca acttggacaa caactcccac |
| 1261 | aagaagaaca tctttgagaa acctttcatg gctcgctatg tgcgtgtcct tccactgtcc |
| 1321 | tggcataacc gtatcaccct gcgcctggag ctgctgggct gttagtgccc agtccttcca |
| 1381 | gcccaagtga cgaggacggc cagaggctga ggggcctcct ggccctgcct cccaggccct |
| 1441 | gctgccttct gtggctgaca ccttctcaat cctccctctt gattgcactg ggactacagg |
| 1501 | caggaagggc aagggggttt cagagttgcc cctcaccctt cccctcaccc tgcagccccc |
| 1561 | acaggcctcc tgctagcccc ttctctcagg cattctgggg gagttggaca ggtctgagat |
| 1621 | gaatagagaa gaagagtgaa gttggggtat gtgggctgct gtaccaacca ccccaagtcc |
| 1681 | taaactttct ccaggggttg actcaagact aaagggaacc tctggttgcc cacccgtctc |
| 1741 | tgcacaccgc acatccctcc atgttccatt cctggaagga gaggcccacg tccgcttgct |
| 1801 | gccccttggg tcaccagatc ctgcctctta tctcctgaga cccctcttga ccctcgctct |
| 1861 | ggagcctcgg ttgacaagag gactgtcggg tctggagaga tagatgggct ctgggtggtt |
| 1921 | ggcgagctgg ctatgggacc tctgctggct tgctacccaa gctaacaagc agattccaaa |
| 1981 | atacatttgt gctctccact ggaaaaaaaa aaaaaaaaaa |

SEQ ID NO: 16 Rat lactadherin isoform 2 (NP_036943.1)

| | |
|---|---|
| 1 | mqfsrvlaal cgvllcasgl faasgdfcds slclnggtcl mgqdndiycl cpegftglvc |
| 61 | netekgpcsp npcfhdakcl vtedtqrgdi fteyicqcpv gysgihcelg cstklglegg |
| 121 | aiadsqisas svymgfmglq rwgpelarly rtgivnawta ssydskpwiq vdflrkmrvs |
| 181 | gvmtqgasra graeylktfk vaysldgrrf efiqdesgtg dkefmgnqdn nslkinmfnp |
| 241 | tleagyirly pvschrgctl rfellgcelh gcseplglkn ntipdsqita sssyktwnlr |
| 301 | afgwyphlgr ldnqgkinaw taqsnsakew lqvdlgtqkk vtgiitqgar dfghlqyvas |
| 361 | ykvahsddgv qwtvyeeqgt skvfqgnldn nshkknifek pfmaryvrvl plswhnritl |
| 421 | rlellgc |

SEQ ID NO: 17 Mouse milk fat globule-EGF factor 8 protein (Mfge8),
transcript variant 2, cDNA (NM_001045489.1, CDS from 94 to 1374)

| | |
|---|---|
| 1 | ggcgcctgat ttattccgga gtgagaggag cggacgcagg aactctcgag tcccagcatc |
| 61 | agagcgcgtg gaccttttcc cgcgtcccgc agcatgcagg tctcccgtgt gctggccgcg |
| 121 | ctgtgcggca tgctactctg cgcctctggc ctcttcgccg cgtctggtga cttctgtgac |
| 181 | tccagcctgt gcctgaacgg tggcacctgc ttgacgggcc aagacaatga catctactgc |
| 241 | ctctgccctg aaggcttcac aggccttgtg tgcaatgaga ctgagagagg accatgctcc |
| 301 | ccaaaccctt gctacaatga tgccaaatgt ctggtgactt tggacacaca gcgtggggac |
| 361 | atcttcaccg aatacatctg ccagtgccct gtgggctact cgggcatcca ctgtgaaacc |
| 421 | ggttgttcta cacagctggg catggaaggg ggcgccattg ctgattcaca gatttccgcc |
| 481 | tcgtctgtgt atatgggttt catgggcttg cagcgctggg gcccggagct ggctcgtctg |
| 541 | taccgcacag ggatcgtcaa tgcctggaca gccagcaact atgatagcaa gccctggatc |
| 601 | caggtgaacc ttctgcggaa gatgcgggta tcaggtgtga tgacgcaggg tgccagccgt |
| 661 | gccgggaggg cggagtacct gaagaccttc aaggtggctt acagcctcga cggacgcaag |
| 721 | tttgagttca tccaggatga aagcggtgga gacaaggagt ttttgggtaa cctggacaac |
| 781 | aacagcctga aggttaacat gttcaacccg actctggagg cacagtacat aaagctgtac |
| 841 | cctgtttcgt gccaccgcgg ctgcaccctc cgcttcgagc tcctgggctg tgagttgcac |
| 901 | ggatgttctg agcccctggg cctgaagaat aacacaattc ctgacagcca gatgtcagcc |
| 961 | tccagcagct acaagacatg gaacctgcgt gcttttggct ggtaccccca cttgggaagg |
| 1021 | ctggataatc agggcaagat caatgcctgg acggctcaga gcaacagtgc caaggaatgg |
| 1081 | ctgcaggttg acctgggcac tcagaggcaa gtgacaggaa tcatcaccca gggggcccgt |
| 1141 | gactttggcc acatccagta tgtggcgtcc tacaaggtag cccacagtga tgatggtgtg |
| 1201 | cagtggactg tatatgagga gcaaggaagc agcaaggtct tccagggcaa cttggacaac |
| 1261 | aactcccaca agaagaacat cttcgagaaa cccttcatgg ctcgctacgt gcgtgtcctt |
| 1321 | ccagtgtcct ggcataaccg catcaccctg cgcctggagc tgctgggctg ttaatgctca |
| 1381 | gtcctgccag cccaaacgat gaggatggcc agaggctgag gggcctcctg gccctgcctc |
| 1441 | ccaggccctg ctgccttctg tggctgacga ccttcttggc cttcccttct gattgtactg |
| 1501 | gggctggagg caggaagggc caggggattt cagagttgcc cttcaccctt tccctcaccc |
| 1561 | tgcagccccc acaggcctcc tgctagcccc cttctctcag gcattctggg ggagttggac |
| 1621 | aggtctgaga tgaatagaga agaagagtga agttggggta tgtgggctat ctgtaccaac |
| 1681 | caccccaagt cctaaacttc ctgccagggc ttgactcagg actgaaggga gcccctgact |
| 1741 | gcccatccct ctctgcacac cacacattcc tccatgttcc attccgggaa ggagaggccc |
| 1801 | acgtccgctt gctgtccctt gggtcaccag gtcctgcctc ttatctcctg agacgcctct |
| 1861 | tgacccttgc actggagcct cagttgacaa ggagactggc gggtctggag aggtcggtgg |
| 1921 | ctctgggtgg ttgacaggtt ggctgtggga cctctgctgg cttgctaccc aagttaacaa |
| 1981 | gcagattcca aaatacattc gtgttctcca ctggaaaaaa aaaaaaaaaa aa |

SEQ ID NO: 18 Mouse lactadherin isoform 2 (NP_001038954.1)

| | |
|---|---|
| 1 | mqvsrvlaal cgmllcasgl faasgdfcds slclnggtcl tgqdndiycl cpegftglvc |
| 61 | netergpcsp npcyndakcl vtldtqrgdi fteyicqcpv gysgihcetg cstqlgmegg |
| 121 | aiadsqisas svymgfmglq rwgpelarly rtgivnawta snydskpwiq vnllrkmrvs |
| 181 | gvmtqgasra graeylktfk vaysldgrkf efiqdesggd keflgnldnn slkvnmfnpt |
| 241 | leagyiklyp vschrgctlr fellgcelhg cseplglknn tipdsqmsas ssyktwnlra |
| 301 | fgwyphlgrl dnqgkinawt aqsnsakewl qvdlgtqrqv tgiitqgard fghiqyvasy |
| 361 | kvahsddgvq wtvyeeqgss kvfqgnldnn shkknifekp fmaryvrvlp vswhnritlr |
| 421 | lellgc |

SEQ ID NO: 19 Mouse milk fat globule-EGF factor 8 protein (Mfge8),
transcript variant 1, cDNA (NM 008594.2, CDS from 94 to 1485)

| | |
|---|---|
| 1 | ggcgcctgat ttattccgga gtgagaggag cggacgcagg aactctcgag tcccagcatc |
| 61 | agagcgcgtg gaccttttcc cgcgtcccgc agcatgcagg tctcccgtgt gctggccgcg |
| 121 | ctgtgcggca tgctactctg cgcctctggc ctcttcgccg cgtctggtga cttctgtgac |
| 181 | tccagcctgt gcctgaacgg tggcacctgc ttgacgggcc aagacaatga catctactgc |
| 241 | ctctgccctg aaggcttcac aggccttgtg tgcaatgaga ctgagagagg accatgctcc |
| 301 | ccaaaccctt gctacaatga tgccaaatgt ctggtgactt tggacacaca gcgtggggac |
| 361 | atcttcaccg aatacatctg ccagtgccct gtgggctact cgggcatcca ctgtgaaacc |
| 421 | gagaccaact actacaacct ggatggagaa tacatgttca ccacagccgt ccccaatact |
| 481 | gccgtcccca ccccggcccc cacccccgat ctttccaaca acctagcctc ccgttgttct |
| 541 | acacagctgg gcatggaagg gggcgccatt gctgattcac agatttccgc ctcgtctgtg |
| 601 | tatatgggtt tcatgggctt gcagcgctgg ggcccggagc tggctcgtct gtaccgcaca |
| 661 | gggatcgtca atgcctggac agccagcaac tatgatagca agccctggat ccaggtgaac |
| 721 | cttctgcgga agatgcgggt atcaggtgtg atgacgcagg gtgccagccg tgccgggagg |
| 781 | gcggagtacc tgaagacctt caaggtggct tacagcctcg acggacgcaa gtttgagttc |
| 841 | atccaggatg aaagcggtgg agacaaggag tttttgggta acctggacaa caacagcctg |
| 901 | aaggttaaca tgttcaaccc gactctggag gcacagtaca taaagctgta ccctgtttcg |
| 961 | tgccaccgcg gctgcaccct ccgcttcgag ctcctgggct gtgagttgca cggatgttct |
| 1021 | gagcccctgg gcctgaagaa taacacaatt cctgacagcc agatgtcagc ctccagcagc |
| 1081 | tacaagacat ggaacctgcg tgcttttggc tggtaccccc acttgggaag gctggataat |
| 1141 | cagggcaaga tcaatgcctg gacggctcag agcaacagtg ccaaggaatg gctgcaggtt |
| 1201 | gacctgggca ctcagaggca agtgacagga atcatcaccc agggggcccg tgactttggc |
| 1261 | cacatccagt atgtggcgtc ctacaaggta gcccacagtg atgatggtgt gcagtggact |
| 1321 | gtatatgagg agcaaggaag cagcaaggtc ttccagggca acttggacaa caactcccac |
| 1381 | aagaagaaca tcttcgagaa acccttcatg gctcgctacg tgcgtgtcct tccagtgtcc |
| 1441 | tggcataacc gcatcaccct gcgcctggag ctgctgggct gttaatgctc agtcctgcca |
| 1501 | gcccaaacga tgaggatggc cagaggctga ggggcctcct ggccctgcct cccaggccct |
| 1561 | gctgccttct gtggctgacg accttcttgg ccttcccttc tgattgtact ggggctggag |
| 1621 | gcaggaaggg ccaggggatt tcagagttgc ccttcaccct ttccctcacc ctgcagcccc |
| 1681 | cacaggcctc ctgctagccc ccttctctca ggcattctgg gggagttgga caggtctgag |
| 1741 | atgaatagag aagaagagtg aagttggggt atgtgggcta tctgtaccaa ccaccccaag |
| 1801 | tcctaaactt cctgccaggg cttgactcag gactgaaggg agcccctgac tgcccatccc |
| 1861 | tctctgcaca ccacacattc ctccatgttc cattccggga aggagaggcc cacgtccgct |
| 1921 | tgctgtccct tgggtcacca ggtcctgcct cttatctcct gagacgcctc ttgacccttg |
| 1981 | cactggagcc tcagttgaca aggagactgg cgggtctgga gaggtcggtg gctctgggtg |
| 2041 | gttgacaggt tggctgtggg acctctgctg gcttgctacc caagttaaca agcagattcc |
| 2101 | aaaatacatt cgtgttctcc actggaaaaa aaaaaaaaaa aaa |

SEQ ID NO: 20 Mouse lactadherin isoform 1 (NP_032620.2)

| | |
|---|---|
| 1 | mqvsrvlaal cgmllcasgl faasgdfcds slclnggtcl tgqdndiycl cpegftglvc |
| 61 | netergpcsp npcyndakcl vtldtqrgdi fteyicqcpv gysgihcete tnyynldgey |
| 121 | mfttavpnta vptpaptpdl snnlasrcst qlgmeggaia dsqisassvy mgfmglqrwg |
| 181 | pelarlyrtg ivnawtasny dskpwiqvnl lrkmrvsgvm tqgasragra eylktfkvay |
| 241 | sldgrkfefi qdesggdkef lgnldnnslk vnmfnptlea qyiklypvsc hrgctlrfel |
| 301 | lgcelhgcse plglknntip dsqmsasssy ktwnlrafgw yphlgrldnq gkinawtaqs |
| 361 | nsakewlqvd lgtqrqvtgi itqgardfgh iqyvasykva hsddgvqwtv yeeqgsskvf |
| 421 | qgnldnnshk knifekpfma ryvrvlpvsw hnritlrlel lgc |

*Included in Table 1 are RNA nucleic acid molecules (e.g., thymines replaced with uridines), nucleic acid molecules encoding orthologs of the encoded proteins.
*Also included in Table 1 are orthologs of the proteins.
Unless otherwise indicated, the practice of the disclosure employs conventional techniques of cell biology, cell culture, molecular biology, transgenic biology, microbiology, recombinant DNA, and immunology, which are within the skill of the art.
The following exaples are offered by way of illustration and not by way of limitation.

### EXAMPLES

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary embodiments, but should be defined only in accordance with the following claims and their equivalents.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

### Example 1: Materials and Methods

### Rodent Fibrosis Model Induction and Efficacy Tests of NP-011

To compare the efficacy of different derivatives of MFG-E8 (NP-011, NP-012, and NP-013) and the full-length protein, MFG-E8, 200 mg/kg of TAA (thioacetamide, Sigma-Aldrich, St. Louis, MO, USA) was administrated into 5- to 6-week-old male C57BL/6 mice (n=4 for each group, 3 times per week for 8 weeks) and each 160 μg/kg of proteins were administrated intraperitoneally. The mice were sacrificed at 3 days after administration of proteins for analysis. Various doses of NP-011 (20 μg/kg ˜160 μg/kg, n=5 for each group) were further tested in the same TAA-induced liver fibrosis model. To test anti-fibrotic effects of multiple administrations of NP-011, TAA was injected into the mice for 12 weeks, and then 40 μg/kg of NP-011 was intraperitoneally administered into the TAA-induced liver fibrosis model one to six times with a 5-day interval (n=4 for each group). For the progressive liver fibrosis model, TAA was injected into the mice for 4 weeks, then 40 μg/kg of NP-011 and TAA were intraperitoneally co-administered into the mice 3 times a week for another 4 weeks (n=5 for each group). In all animal experiments, the mice were sacrificed three days after the last NP-011 administration: all experimental procedures were approved by the Institutional Animal Care and Use Committee of Korea University (KOREA-2016-0254).

### DMN-Induced Cirrhosis Model Induction and Efficacy Tests of NP-011

Animal model for liver cirrhosis was designed and conducted by National Center of Efficacy Evaluation for the Development of Health Products Targeting Digestive Disorders (NCEED, Incheon, Korea). Liver cirrhosis was induced by intraperitoneal injection of dimethylnitrosamine (DMN in saline, 10 mg/ml/kg) for 3 consecutive days each week during a 6-week period using male 6-week-old Sprague-Dawley rats (Orient Bio, Gapyeong, Korea). To minimize the body weight deviation between groups, at the first day of the experiment and the week 3, we randomly separated rats two times into 7 groups: G1 (vehicle control, n=10), G2 (fibrosis control, n=12), G3 (NP-011 treated, once a week, n=10), G4 (NP-011 treated, twice a week, n=10), G5 (NP-011 treated, three times a week, n=10), and G6 (NP-011 treated, daily, n=10). Administration of NP-011 was initiated from the week 3 using intravenous (G3-G6) with 40 μg/kg intravenously. General observation was conducted every day and body weight was measured twice for a week. At the end of week 6, all groups of surviving rats were sacrificed for autopsy and calculated the survival rate of each group. Rats were anesthetized with isoflurane and collected blood sample and main organs. After fixing the liver tissue in 10% neutral-buffered formalin solution, histologic assessment and immunohistochemistry were performed with hematoxylin & eosin staining and α-smooth muscle actin antibody by PostBio Inc., (Guri, Korea).

### MCD Diet-Induced NASH Mouse

6-weeks-old male C57BL/6 mouse fed standard chow or Methionine and choline-deficient (MCD) diet for 10 weeks. The procedures employed for the handling and care of the animals were approved by the Korea University Institutional Animal Care and Use Committee (KUIACUC-2018-3). After 5 weeks, mice were intraperitoneally injected with saline or NP-011 (20 μg, 40 μg, 80 μg, or 160 μg/kg body weight, once a week, n=5) for more 5 weeks with MCD diet. During the experiment, body weight was measured weekly.

### Immunofluorescence Assay

The liver tissues were fixed in 4% paraformaldehyde (PFA) and dehydrated in a graded ethanol series. The tissues were then cleared in xylene and embedded in paraffin. Paraffin-embedded tissue sections were stained with Sirius red (American MasterTech Scientific, Lodi, CA, USA) for the evaluation of liver fibrosis. For immunofluorescence staining, sectioned tissues underwent antigen retrieval with citric acid and tissues blocked with 10% donkey serum containing PBS were probed with the primary antibody against alpha-smooth muscle actin (α-SMA) or albumin (ALB) at 4° C. overnight. For visualization of the staining, the sections were washed with 0.1% bovine serum albumin (BSA) containing phosphate buffered saline (PBS) and stained with fluorescently labeled secondary antibodies (Invitrogen/Thermo Fisher Scientific, Carlsbad, CA, USA). Digital images were captured using a microscope (Nikon, Tokyo, Japan) and analyzed using ImageJ software.

### Histopathological Analysis and NAS Score

Liver tissue was fixed in 4% paraformaldehyde, embedded in paraffin, sectioned and stained with hematoxylin and eosin (H&E) according to a standard procedure. Histological scoring of the liver lesions was assessed by grading system for rodent NAFLD, as described (1): Macrovesicular steatosis (0-3), microvesicular steatosis (0-3), hypertrophy (0-3), numbers of inflammatory foci/field (0-3). All cells of randomly picked five images were counted for each histological feature and calculated. For Oil Red O staining, OCT-embedded frozen livers were sectioned at 4 μm, and stained with Oil Red O staining kit (Lifeline cell technology, Frederick, MD, USA, Cat.LL-0052). Oil Red O stained area were measured by Image J. For immunostaining of frozen-sections, OCT-embedded frozen livers were sectioned at 4 μm, slides were permeabilized and masking by 0.1% Triton X-100 and 10% donkey serum. The liver sections were incubated with primary and secondary antibodies specified in Table 2.

### TGF-β Luciferase Signaling Reporter Assay

The luciferase signaling reporter array was performed according to the manufacturer's protocol (Qiagen, Hilden, Germany). The detailed methods for whole transcriptome analysis was described in supplementary materials and methods.

### Whole Transcriptome Analysis of Mouse Livers

The normal mouse livers (n=3), TAA-induced fibrotic livers (n=3), and NP-011-administrated livers (n=3) were homogenized in cold Trizol (Sigma, USA), and next generation sequencing (NGS) analysis was performed by BGI Tech Solutions (Hong Kong). Gene Set Enrichment Analysis (GSEA) analysis was conducted using GSEAv17 (Broad Institute, Cambridge, MA, USA). The detailed methods for whole transcriptome analysis was described in supplementary materials and methods.

### Quantitative Reverse-Transcription PCR (RT-qPCR)

Quantitative polymerase chain reaction (qPCR) was performed using the CFX96 real-time PCR detection system (Bio-Rad, Hercules, CA, USA) with iQ™ SYBRR Green Supermix (Bio-Rad). The specific primers used are provided in Table 3. The mRNA levels were normalized to the level of GAPDH (glyceraldehyde-3-phosphate dehydrogenase).

### Cell Culture and Reagents

The human hepatic stellate cell (HSC) line, hTERT-HSC, was cultured in Dulbecco's modified Eagle's medium (DMEM; GE Healthcare Life Sciences, Marlborough, MA, USA) supplemented with 10% fetal bovine serum (FBS; Gibco/Thermo Fisher Scientific, Waltham, MA, USA), 100 U/mL penicillin, and 100 mg/mL streptomycin (Gibco). The human HEK-293 FT cells kindly provided from Prof. Hyunggee Kim (Korea university) were cultured in Dulbecco's modified Eagle's medium (DMEM; GE Healthcare Life Sciences, IL, USA) supplemented with 10% fetal bovine serum (FBS; Gibco, NY, USA), 100 U/mL of penicillin and 100 mg/mL of streptomycin (Gibco).

### Western Blot and Immunoprecipitation (IP) Analysis

Protein samples were prepared by solubilizing HSCs in RIPA lysis buffer (LPS solution) containing proteinase inhibitors (Roche, Basel, Switzerland). A total of 40 μg of protein from cells was separated by SDS-PAGE (Bio-Rad) and transferred to PVDF transfer membranes (Pall Corporation, Port Washington, NY, USA). The membranes were incubated for 60 min with 5% skim milk in TBS-T (10 mM Tris-HCl pH 7.9, 150 mM NaCl, and 0.05% Tween-20) to block nonspecific antibody binding sites. After blocking, the membranes were immunoblotted with primary antibodies overnight at 4° C. Antibodies used in the present study are provided in Table 2. For immunoprecipitation (IP) analysis, a total of 400 μg of protein was incubated at 4° C. for 12 hours with 1 μg of TGFβRI antibody, conjugated to protein A/G sepharose beads (Santa Cruz Biotechnology, Inc., Dallas, TX, USA) washed in lysis buffer, then separated on SDS-PAGE gels. To detect each band in western blot and IP analysis, the membranes were incubated for 2 hours with horseradish peroxidase (HRP)-conjugated secondary antibodies (Thermo Fisher Scientific) at room temperature. After rinsing with TBS-T, the membranes were developed with the Pierce™ ECL western blotting substrate (Thermo Fisher Scientific) to detect bands using a chemiluminescence imaging system (GE Healthcare Life Sciences).

### Efficacy Test of NP-011 in 3D Human Liver Fibrosis Model

In order to establish human liver fibrosis model, the liver spheroids were formed by mixture of hepatocytes (Hepatosight-SR, NEXEL, Seoul, South Korea) and hTert-HSCs (kindly provided from Dr. David Brenner, University of California at San Diego, Ca, USA) in ultra-low attachment 96 well plate (Corning) with the cell density ratio 2 to 1, respectively. The liver spheroids were cultured for 21 days, and 50 mM of acetaminophen (APAP) was treated to induce the fibrosis. To test the efficacy of NP-011 against the APAP-induced 3D liver fibrosis model, 500 ng/ml of NP-011 was treated for 48 hr.

### In Vitro Studies for HSC Activation

To investigate the effects of NP-011 on TGF-β1-mediated HSC activation, human HSC lines (hTERT-HSCs) were grown in the presence of serum, then starved in DMEM containing 0.2% FBS 24 hr before TGF-β1 treatment. The serum-starved HSCs were pre-treated with 10 ng/ml TGF-β1 for 1 hour, and the HSCs were exposed to 100-1500 ng/ml NP-011 for 6 hours. For blocking integrin avβ3/β5 in the HSCs, the HSCs were pre-treated with 1 μM CT (Selleck Chemicals, Houston, TX, USA) for 2 hours before treating with TGF-β1. The activation and deactivation of HSCs was quantitatively determined by 5-ethynyl-2′-deoxyuridine (EdU) assay.

### Proximity Ligation Assay (PLA)

For the PLA incorporation assay, human HSCs were seeded at 2×10⁴ cells per well on the 18 mm circular cover glass in 12-well plates and cultured for 24 hours. After serum starvation for 24 hours, the cells were treated with 10 ng/ml of TGF-β1 and/or 500 ng/ml of NP-011, and then, further incubated for 30 minutes. The PLA incorporation was accessed using the Duolink In Situ Red Starter Kit (Merck) according to the manufacturer's instructions. Digital images of PLA-positive cells were captured using a microscope (Nikon) and analyzed using ImageJ software <https://imagej.nih.gov/ij/>.

### EdU Incorporation Assays

For the EdU incorporation assay, human HSCs were seeded at 2×10⁴ cells per well in 12-well plates and cultured for 24 hr. After treatment with TGF-β1 and/or NP-011, the serum-starved HSCs were incubated with EdU (10 μM) for an additional 6 hours and EdU incorporation was accessed using the Click-iT EdU Imaging Kit (Thermo Fisher Scientific) according to the manufacturer's instructions. Digital images of EdU-positive cells were captured using a microscope (Nikon) and analyzed using ImageJ software.

### Collagenase Activity Assay

The collagenase activity assay was performed according to the manufacturer's protocol (Chondrex, WA, USA). Enzyme sources were mixed with 180 μl of solution B. Enzymatic reaction was initiated by mixing with 200 μl of 1.0 mg/mL FITC-labeled bovine collagen I substrate, followed by incubation at 37° C. for 1 hour. For negative controls, the enzymatic reaction was performed either without the collagen substrate or the enzyme source. To stop the enzymatic reaction, 10 μl of 10 mM o-phenanthroline and 10 μl of 38.5 μM elastase were added to every sample, followed by incubation at 37° C. for 10 mins. Finally, a 400 μl aliquot of extraction buffer was mixed well with the reaction solution, which was then centrifuged at 10,000 rpm for 5 mins. The supernatant (200 μl) was used for the measurement of FL intensity at 520 nm with the spectrofluorometer during 490-nm excitation in black 96 well plate.

### THP-1 Differentiation and Fluorescent Bead Phagocytosis Assay

THP-1 cells were purchased from ATCC and cultured in RPMI-1640 (Gibco, 31800-022) containing 10% of heat inactivated fetal bovine serum (gibco, 16000-044) and 50 μM β-mercaptoethanol in 37° C., 5% CO₂ incubator. THP-1 cells were differentiated into macrophages using 200 ng/ml of phorbol 12-myristate 13-acetate (PMA, Sigma, P8139) for 48 hours followed by 72 hours in PMA-free medium. In phagocytosis assay, THP-1 cells were seeded at 1.05×10⁵ cells/cm². Carboxylate-Modified Microspheres, 2.0 μm, yellow-green fluorescent beads (Invitrogen, F8827) were washed in THP-1 cell culture media and resuspended at a final dilution of 1:500 in serum free RPMI-1640. Cells were incubated with fluorescent beads for 4 hours in 37° C., 5% CO₂ incubator. Cells were detached with TrypLE (Gibco, 12604-021) and measured by flow cytometry (Accuri C6 Plus).

### Biodistribution Study

6-weeks-old, male C57BL/6 mouse were randomized into two groups, the control group (Saline) and the NP-011 treated group (NP-011, 160 μg/kg body weight in saline) with 3 animals for each time-point. Mice were injected intravenously and maintained for different time-points. Mice were sacrificed by CO₂ inhalation, and the organs (brain, heart, lung, liver, kidney, and spleen) were collected directly in PBS to remove the fat and blood traces. The organs stored at −80° C. immediately. The procedures employed for the handling and care of the animals were approved by the Korea University Institutional Animal Care and Use Committee (KUIACUC-2018-0027 and KUIACUC-2018-0040). Whole organs were homogenized by Lysis buffer (RayBiotech, Norcross, GA, USA, Cat.EL-LYSIS) with complete™ Protease Inhibitor Cocktail (Sigma-Aldrich. Cat.11697498001) at 4° C. After homogenization, samples were centrifuged for 20 min at 13,000 rpm at 4° C. and the supernatant was collected for ELISA analysis. The Bio-Rad Bradford was used to calculate protein concentration of each samples. To detect the NP-011, Human MFG-E8 Quantikine ELISA kit (R&D. Cat.DFGE80) was performed following manufacturer's instructions. The result was measured at 450 nm, 540 nm and 570 nm by SpectraMax® iD3 (Molecular Devices. San Jose, CA, USA).

### Repeated Dose 28-Day Chronic Toxicity Study

4-week repeated toxicity study of NP-011 was conducted by ChemOn Inc. (Non-clinical CRO company, Korea, Gyeonggi-DO). Total 30 SD rats (15 males and 15 females, 6 weeks-old) were randomly divided into 3 groups; G1 (vehicle control), G2 (NP-011 0.2 mg/kg, daily), and G3 (NP-011 2.2 mg/kg, daily), each 5 male rats and 5 female rats per group. The vehicle and NP-011 were administrated via tail vein of rates. The body weight and consumption of food and water were measured once before the start and every week during the test in all groups. After the end of the study, all rats were sacrificed with blood sampling for the examination of ophthalmology, urine, hematology, biochemistry, and histopathology. The histopathological assessment was performed in all major organs and area (brain, liver, heart, spleen, lung, kidney, bone marrow-sternum, injection site, testis/uterus, and thymus).

### Statistical Analysis

Numerical values were expressed as the mean+SEM of at least three independent experiments performed in triplicate for in vitro studies. To evaluate the anti-fibrotic effects of NP-011 in vivo, at least three animals per group were used in each experiment and data were obtained from two to three independent experiments unless indicated. The percentages of areas positive for the Sirius red staining or immunostaining of total image area were measured using ImageJ software and expressed as relative values compared to those in normal livers or control cell cultures, which were arbitrarily set as 1. The Student's t-test was used to analyze the statistical significance of differences between the paired groups. One-way analysis of variance (ANOVA) was used to test the statistical significance of differences among multiple groups (more than two groups). The data are expressed as means and 95% confidence intervals (CI). All statistical tests were two-sided and data with P<0.05 or P<0.01 were assumed to be statistically significant.

TABLE 2

| Antibody | Vendor | Application | Designation |
|---|---|---|---|
| β-actin | Abcam | WB | ab25894 |
| p-Smad2 | Abcam | WB | ab188334 |
| Smad2 | Abcam | WB | ab33875 |
| TGFBRI | Abcam | IP | ab31013 |
| Integrin B3 | Abcam | WB, IP | ab25894 |
| Integrin B5 | Cell | WB, IP | #3629 |
| | Signaling | | |
| Mouse α-SMA | Abcam | IF (Frozen) | ab21027 |
| Mouse F4/80 | Bio-rad | IF (Frozen) | MCA497 |
| Human MFG-E8 | R&D | IF (Frozen) | RD27890 |
| Donkey anti-Goat IgG (H + L) | Invitrogen | IF (Frozen) | A11058 |
| Highly Cross-Adsorbed | | | |
| Secondary antibody, | | | |
| Alexa Fluor 594 | | | |
| Donkey anti-Rabbit IgG (H + L) | Invitrogen | IF (Frozen) | A21207 |
| Highly Cross-Adsorbed | | | |
| Secondary antibody, | | | |
| Alexa Fluor 594 | | | |
| Donkey anti-Mouse IgG (H + L) | Invitrogen | IF (Frozen) | A21202 |
| Highly Cross-Adsorbed | | | |
| Secondary antibody, | | | |
| Alexa Fluor 488 | | | |
| Goat anti-Rabbit IgG (H + L) | Invitrogen | WB | 31460 |
| Secondary Antibody, HRP | | | |
| Donkey anti-Sheep IgG (H + L) | Invitrogen | WB | A16041 |
| Secondary Antibody, HRP | | | |
| α-SMA | Santacruz | IF | sc-53142 |
| Donkey anti-Rabbit IgG (H + L) | Invitrogen | IF | A21207 |
| Highly Cross-Adsorbed | | | |
| Secondary Antibody, | | | |
| Alexa Fluor 594 | | | |

TABLE 3

| Species | Primer | Sequences (5′ −> 3′) |
|---|---|---|
| Mouse | Gapdh | F: GTT GTC TCC TGC GAC TTC A |
| | | R: GGT GGT CCA GGG TTT CTT A |
| Mouse | Col1a1 | F: CAA TGC AAT GAA GAA CTG GAC TGT |
| | | R: TCC TAC ATC TTC TGA GTT TGG TGA |
| Mouse | Col1a2 | F: GCA GGG TTC CAA CGA TGT TG |
| | | R: GCA GCC ATC GAC TAG GAC AGA |
| Mouse | Acta2 | F: CTG ACA GAG GCA CCA CTG AA |
| | (αSMA) | R: CAT CTC CAG AGT CCA GCA CA |
| Mouse | Tgfb | F: TGA CGT CAC TGG AGT TGT ACG G |
| | | R: GGT TCA TGT CAT GGA TGG TGC |
| Mouse | Mmp2 | F: AAC TTT GAG AAG GAT GGC AAG T |
| | | R: TGC CAC CCA TGG TAA ACA A |
| Mouse | Mmp9 | F: CTG GAC AGC CAG ACA CTA AAG |
| | | R: CTC GCG GCA AGT CTT CAG AG |
| Human | GAPDH | F: GCT CTC TGC TCC TCC TGT TC |
| | | R: CCA TGG TGT CTG AGC GAT GT |
| Human | MMP2 | F: AGC TCC CGG AAA AGA TTG ATG |
| | | R: CAG GGT GCT GGC TGA GTA GAT |
| Human | MMP9 | F: CAC GCA CGA CGT CTT CCA |
| | | R: AAG CGG TCC TGG CAG AAA T |

### Example 2: NP-011 Inhibits Liver Fibrosis

Human MFG-E8 contains three domains: a signal peptide at the N-terminus, an epidermal growth factor (EGF)-like domain that bears an arginine-glycine-aspartate (RGD) motif, and C domains (C1 and C2). Although it is well known that MFG-E8 modulates inflammatory responses by RGD motif-mediated binding to immune cells and engulfing phosphatidylserine (PS)-expressing apoptotic cells, it is unclear how MFG-E8 is responsible for the anti-fibrotic effect. A recent report showed that the C2 domain of MFG-E8 plays a key role in recognizing PS in apoptotic cells, suggesting the C2 domain may be important for the over all function of MFG-E8, including its anti-fibrotic effect. To test this, it was synthesized herein two different truncated forms of MFG-E8 along with the EGF domain to maintain the RGD motif for cell binding (NP-011: EGF-C1 domain and NP-012: EGF-C2 domain). As a control, full length MFG-E8 (NP-013) was also synthesized.

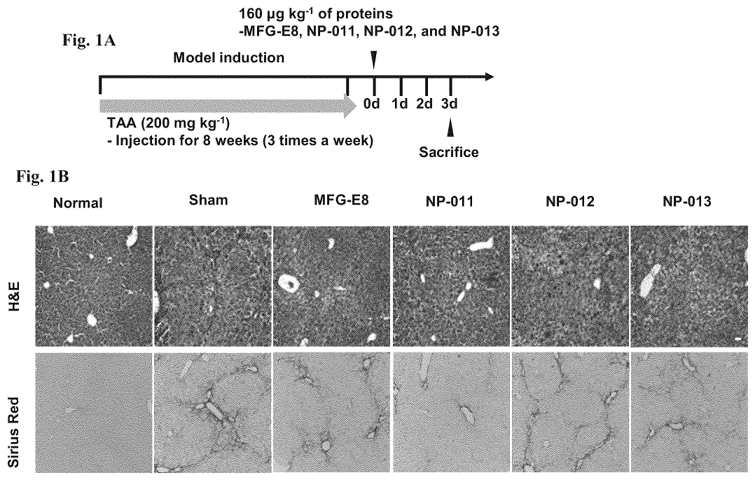

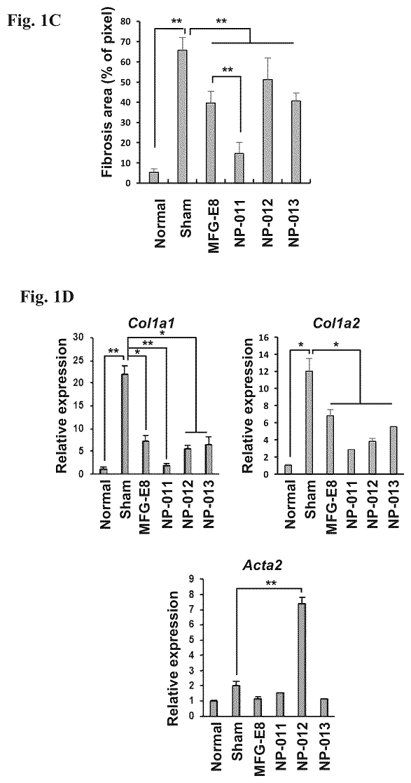

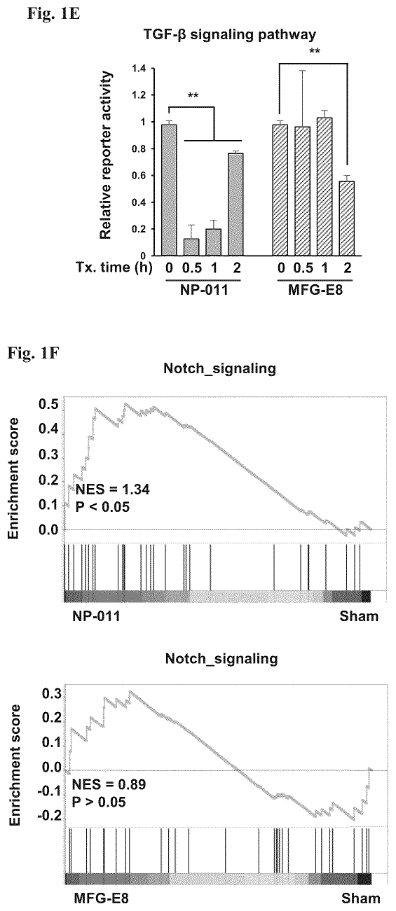

A subsequent efficacy test in a thioacetamide (TAA)-induced liver fibrosis mouse model (FIG. 1A) revealed that the administration of commercial MFG-E8 or NP-013 effectively reduced the fibrotic area (Sirius red-stained area, FIG. 1B and FIG. 1C) and downregulated the expression level of liver fibrosis-related genes (Colla1, Colla2, and Acta2) (FIG. 1D). However, it is compelling that the administration of NP-011 eliminated the fibrotic area more substantially and downregulated the expression level of fibrotic genes in the injured liver more significantly than the administration of MFG-E8 and NP-013 (FIG. 1B to FIG. 1C). This is surprising because NP-011 lacks the C2 domain that was thought to be important for the function of MFG-E8, including the anti-fibrotic effect of MFG-E8. Unlike the strong efficacy of NP-011, the administration of NP-012 was less efficacious in reducing the fibrotic area than MFG-E8 and NP-013 (FIG. 1B and FIG. 1C) and significantly upregulated the expression of Acta2 (alpha smooth muscle actin, α-SMA) in the injured liver (FIG. 1D). Signaling reporter assay revealed that NP-011 rapidly suppressed TGF-β signaling within 30 min whereas MFG-E8 could suppress the TGF-β signaling 2 h after treatment (FIG. 1E). GSEA further identified that the administration of NP-011 significantly enriched the Notch signaling in whole transcriptome analysis of injured liver (FIG. 1F) which is associated with the regeneration processes of injured liver (15). These results indicate that deletion of the C2 domain of MFG-E8 unexpectedly results in more potent anti-fibrotic effect compared to the full-length protein.

### Example 3: NP-011 Significantly Reverses Liver Fibrosis at Minimal Dosage

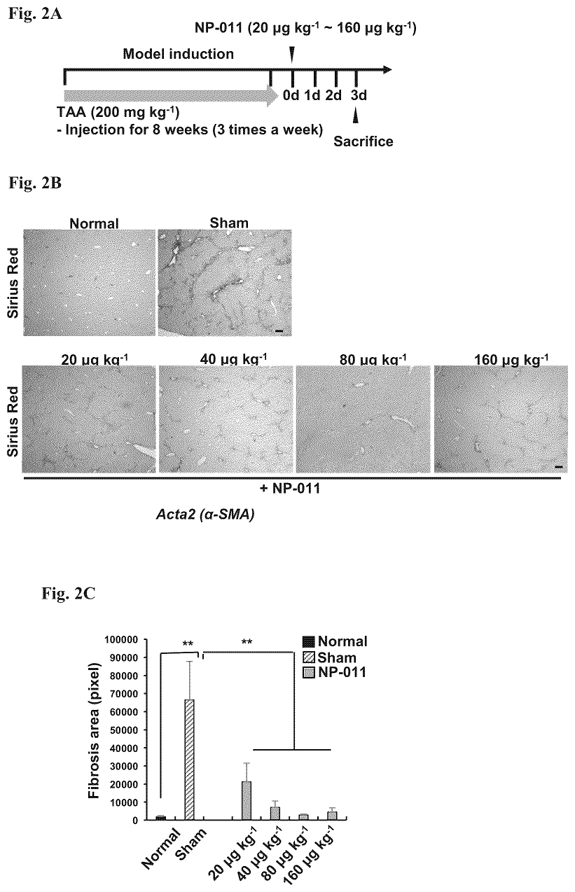

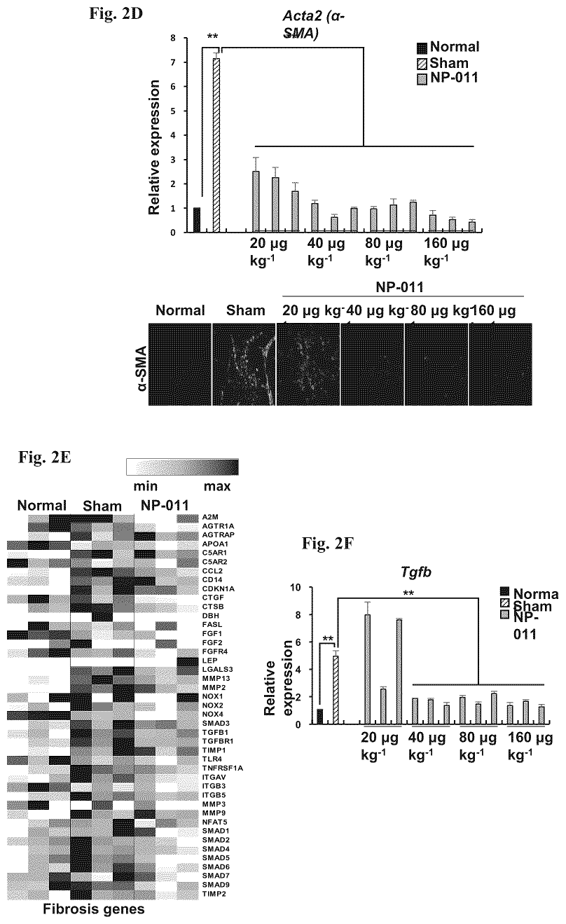

To explore the effective dosage of NP-011, different doses of NP-011 (20 μg/kg, 40 μg/kg, 80 μg/kg, and 160 μg/kg) were administered in a TAA-induced liver fibrosis model and analyzed the fibrotic factors 3 days after NP-011 administration (FIG. 2A). The administration of TAA significantly increased the fibrosis area in the mouse liver (FIG. 2B and FIG. 2C, Sham). In contrast to sham-treated liver tissues, all the NP-011-administrated groups ranging from 20 to 160 μg/kg showed remarkably diminished fibrosis area (FIG. 2B and FIG. 2C). Consistent with the decrease in fibrosis, a key fibrosis marker, Acta2 (α-SMA, a marker for myofibroblast-like cells differentiated from HSCs), and other fibrosis-related genes were markedly and significantly downregulated at both mRNA and protein levels after NP-011 administration (FIG. 2D and FIG. 2E). It is noteworthy that the administration of higher doses than 40 μg/kg resulted in the stable and profound suppression of TGF-β mRNA expression with slight individual variations (FIG. 2F). Taken together, these results imply that NP-011 shows great therapeutic efficacy at low dosage ranges: most effectively, the administration of 40 μg/kg of NP-011 might be the minimum efficacious dose, resulting in a constant reduction of TGF-β expression in injured liver.

### Example 4: The Minimum Dose of NP-011 Shows Therapeutic Efficacy in Different Models Associated with Fibrosis

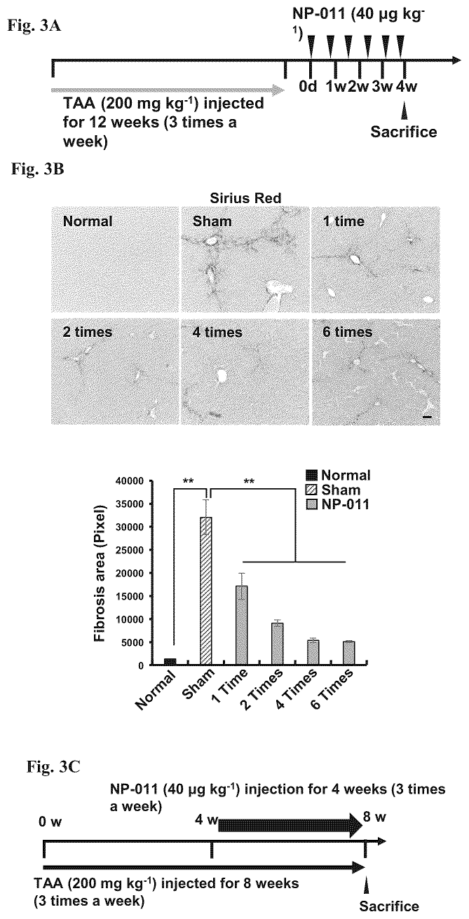

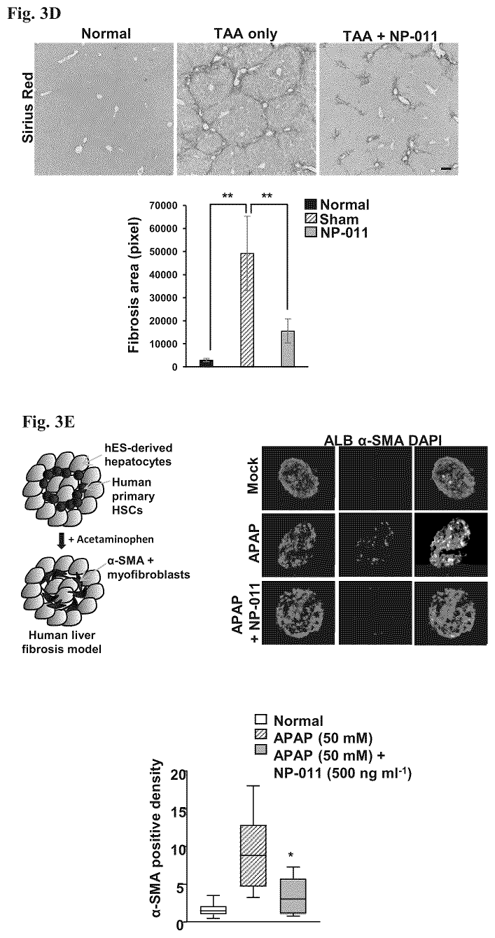

The minimum efficacious dose of NP-011 (40 μg/kg) was further tested in different liver fibrosis models. Firstly, the efficacy of repeated administrations of NP-011 was tested in chronic model of liver fibrosis. For this, TAA injections were extended from 8 to 12 weeks (3 times a week), and then 40 μg/kg of NP-011 was administered to the mice one to six times with 5-day intervals (FIG. 3A). The 12-week TAA injections resulted in the development of sustained fibrosis areas in the liver despite the TAA injections were halted 30 days before sacrificing the mice (FIG. 3B, sham). In contrast, the administration of NP-011 significantly resolved the fibrotic areas in injured livers and the fibrotic regression was positively correlated with the times of administration (FIG. 3B).

Liver fibrosis is a progressive disease and thus evaluation of the efficacy of NP-011 on progressing liver fibrosis may provide a better translation of the animal study to the clinical realm. To confirm the therapeutic efficacy of NP-011 in the progressing liver fibrosis, the NP-011 (40 μg/kg, three times a week) was concurrently administrated with TAA for last 4 weeks of model induction (FIG. 3C). As expected, the concurrent administrations of TAA and NP-011 markedly diminished the fibrosis areas in the livers, compared with the liver that received TAA only (FIG. 3D). We also used APAP-induced in vitro human fibrosis model and found that NP-011 significantly reduced APAP-induced HSC activation in 3D hepatic spheroids consisting of Hepatocytes and hepatic stellate cells (FIG. 3E).

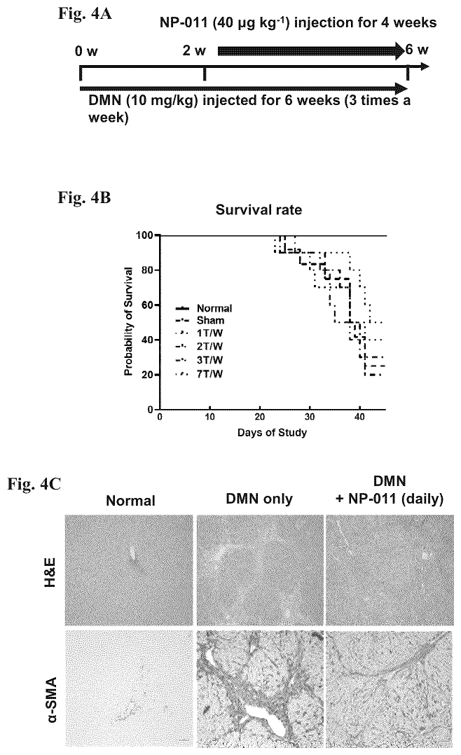

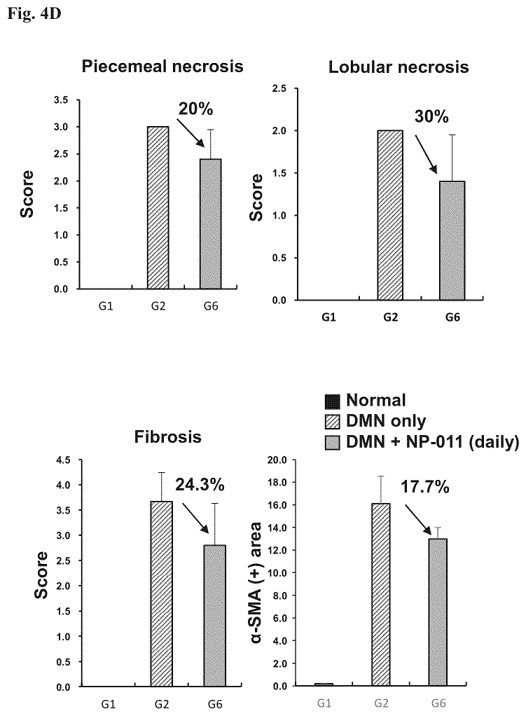

The efficacy of NP-011 was further tested in dimethylnitrosamine (DMN)-induced liver cirrhosis model (FIG. 4A) (16). The DMN injection for 6 weeks in rats resulted the 75% lethality with severe fibrosis in the liver (FIG. 4B and FIG. 4C). Histopathological analysis showed that significant necrotic (piecemeal and lobular) and fibrotic events occurred in the liver of DMN-induced cirrhosis model (FIG. 4D). Importantly, daily administrations of NP-011 reduced the lethality of animals down to 50% with profound attenuation of the liver fibrosis (FIG. 4B and FIG. 4C), and also reduced piecemeal necrosis (˜20%), lobular necrosis (˜30%), fibrosis score (˜24.3%) and HSC activation (˜17.7%) (FIG. 4D).

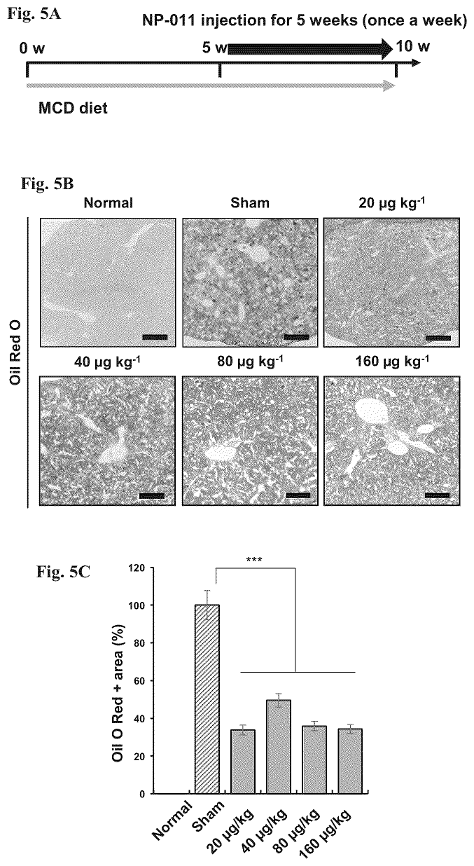

Finally, the efficacy of NP-011 was assessed in methionine-choline deficient (MCD) diet-induced NASH model (FIG. 5A). As reported in various research, MCD diet resulted fat accumulation in the liver, but administrations of NP-011 significantly reduced the fat accumulations in all tested doses (FIG. 5B and C). Furthermore, significant decreases in the macro-/micro-vesicular steatosis and hypertrophy were observed following NP-011 administration (FIG. 5D). Taken together, NP-011 showed therapeutic efficacies, not only in the severe liver fibrosis but also in NASH at minimum dose.

### Example 5: NP-011 Resolves Liver Fibrosis by Interaction with Hepatic Stellate Cells (HSCS) and Macrophages in Injured Liver

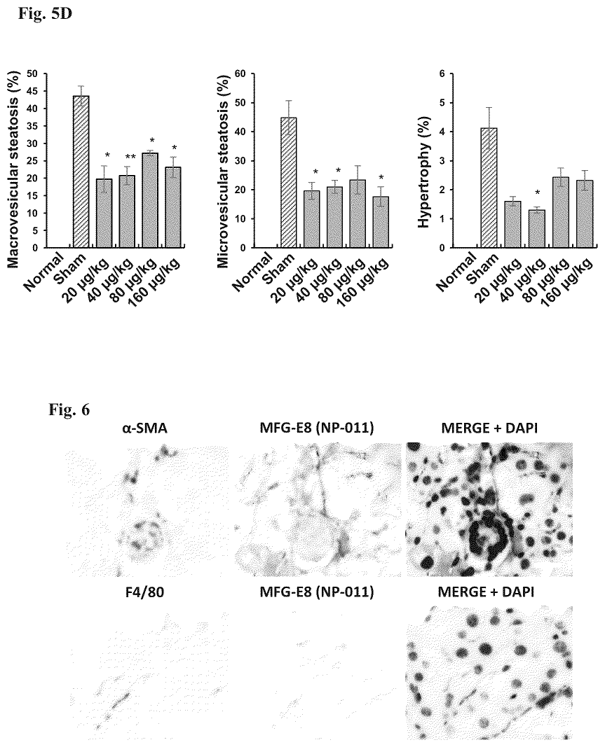

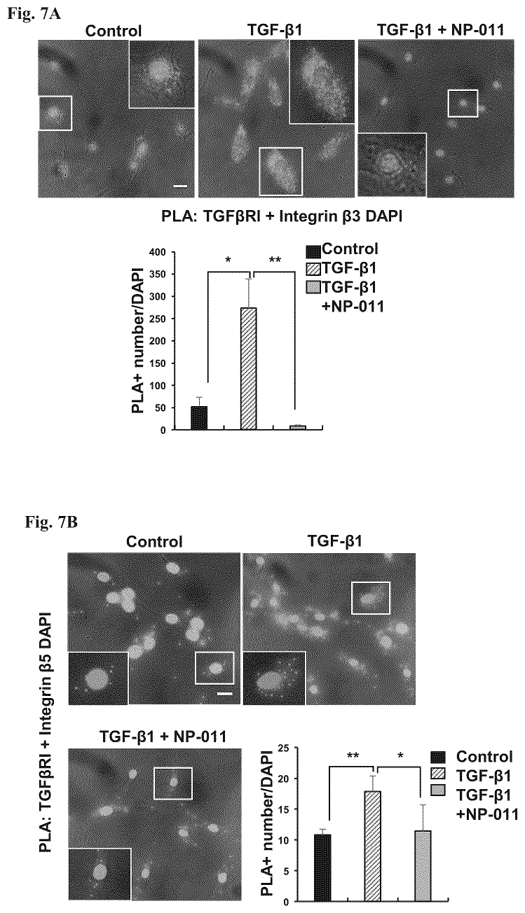

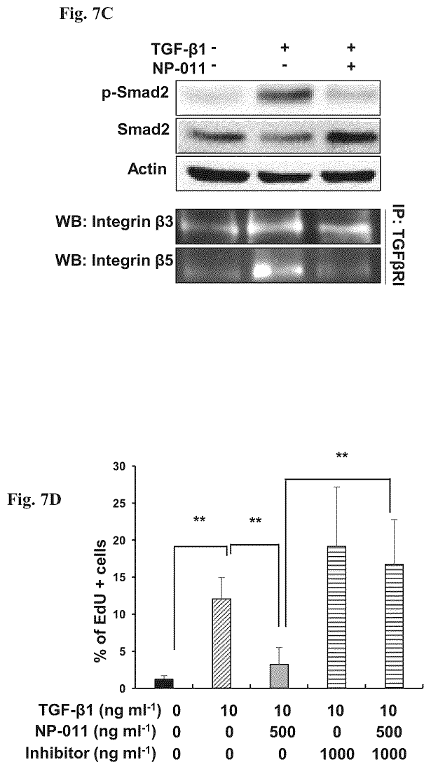

It was investigated herein the mechanism of action for NP-011's rapid and effective suppression TGF-β signaling observed in diseased liver (FIG. 1E and 2F). One day after administration of NP-011 in TAA-induced fibrotic liver. NP-011 preferentially bound to α-SMA-positive HSCs, and some of NP-011 interacted with macrophages in the liver (FIG. 6). Integrin 60 vβ3 and αvβ5 on the surface of HSCs have been previously implicated in regulating fibrosis. Additionally, the crosstalk between integrin and TGF-β signaling may be important in the regulation of pathological epithelial to mesenchymal transition (EMT) and myofibroblast differentiation. Proximity Ligation Assay (PLA) revealed direct physical interactions between TGFBRI and integrin β3/β5, and the interactions became stronger upon TGF-β1 treatment in HSCs (FIG. 7A and FIG. 7B). However, NP-011 treatment in TGF-β1-treated HSCs significantly decreased the interactions between TGFBRI and integrin β3/β5 (FIG. 7A and FIG. 7B). Western blot analysis showed that NP-011 treatment significantly attenuated the phosphorylation of a downstream molecule of TGF-β signaling. Smad2, in TGF-β1-treated HSCs, and the interactions between TGFBRI and integrin β3/β5 were further confirmed by immunoprecipitation assay (FIG. 7C). As a consequences of NP-011 treatment in TGF-β1-treated HSCs, the proliferative HSCs returned to quiescent status (FIG. 7D). However, treatment of integrin β3/β5 inhibitor in the presence of NP-011 abrogated the suppressive role of NP-011 in the proliferation of TGF-β1-treated HSCs (FIG. 7D). Therefore, these results indicate that NP-011 directly binds to integrin β3/β5 and interferes with its interaction with TGFβRI, resulting in suppression of the TGF-β cascade and decrease in HSC proliferation.

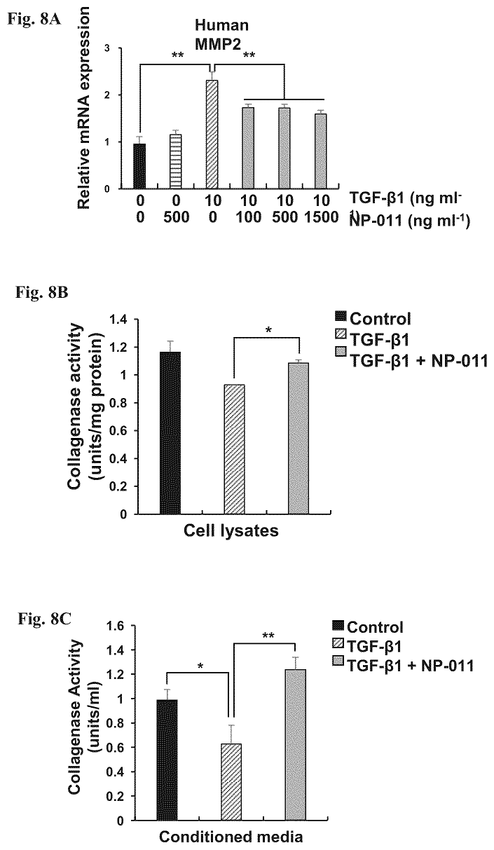

Effects of NP-011 in the expression of pro-fibrotic MMP2 and collagenase activity in HSCs were further tested based on the previous finding that showed secretion of pro-fibrotic MMP2 and expression of collagenase mRNA in rat HSCs. Expression of MMP2 mRNA in HSCs was increased in HSCs after TGF-β1 treatment. However, NP-011 treatment in TGF-β1-treated HSCs significantly down-regulated the increased expression of MMP2 (FIG. 8A). In contrast, the collagenase activity was down-regulated in HSCs after TGF-β1 treatment, and the decreased collagenase activity was recovered in TGF-β1-treated HSCs after NP-011 treatment both in cell lysates and conditioned media (FIG. 8B and C).

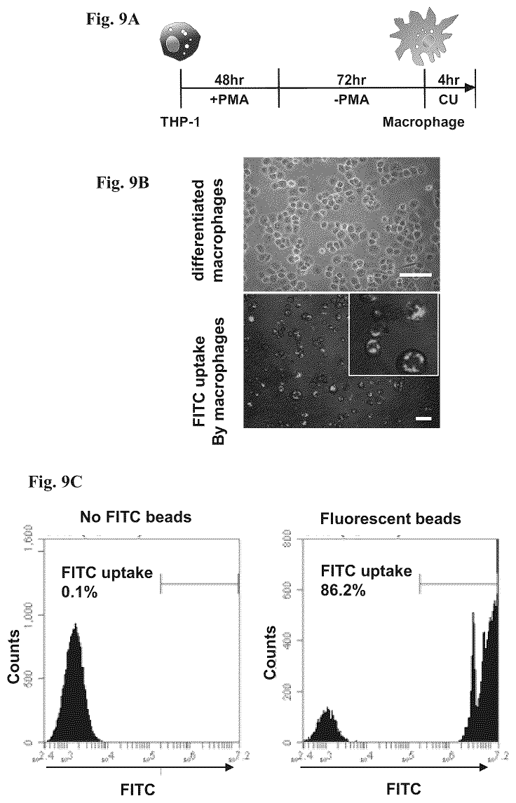

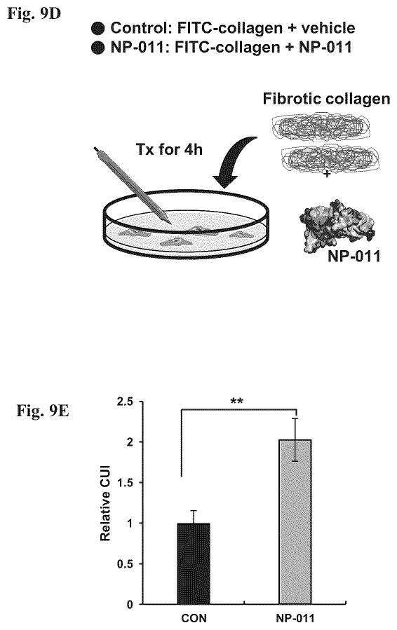

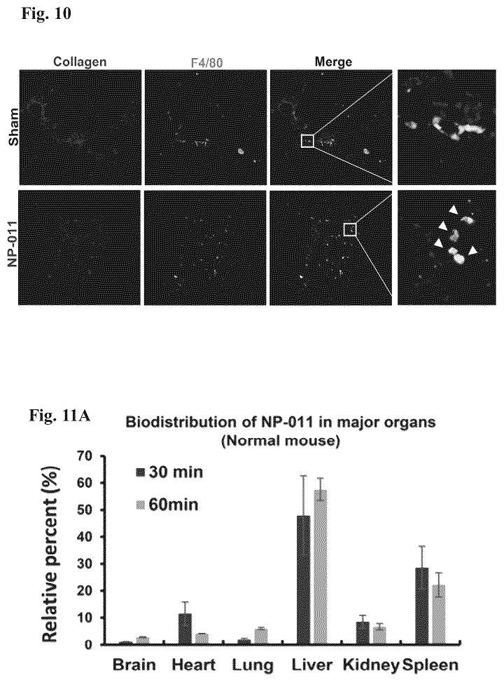

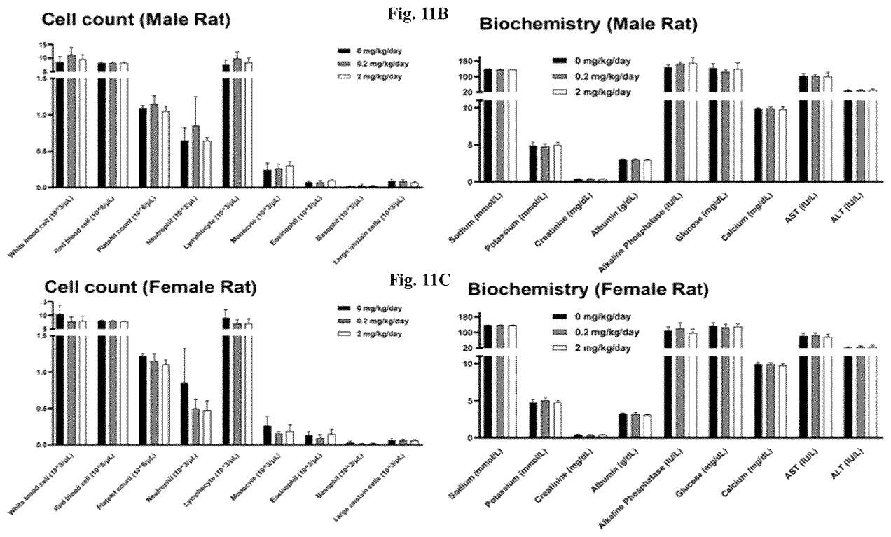

Elimination of accumulated collagen by immune response is another key factor for resolving fibrosis as seen in lung fibrosis model. In order to assess this, THP-1 monocytes were differentiated into macrophages by PMA treatment (FIG. 9A), and the phagocytic capability of differentiated macrophages was confirmed by fluorescence and flow cytometry analysis (FIG. 9B and C). Collagen uptake assay revealed that green fluorescence-labelled collagen was significantly up-taken by differentiated macrophages in the presence of NP-011 unlike the control culture (FIG. 9D and E). In fact, engulfment of collagen by macrophages was demonstrated by immunostaining for collagen and F4/80 after NP-011 administration in TAA-induced liver fibrosis model (FIG. 10, arrow heads in lower panel). Taken together, these results indicate that that NP-011 suppresses TGF-β signaling in injured liver, and then inhibits and resolves liver fibrosis by deactivating active HSCs and interacting with macrophages.

### Example 6: Bio-Distribution and Safety Profiles of NP-011

Bio-distribution and safety test of NP-011 were further assessed in a rodent model. When the NP-011 was intravenously administrated into mice, the administrated NP-011 was preferentially delivered into liver, and then about 48% and 58% of administrated NP-011 were detected in the liver within 30 min and 60 min, respectively (FIG. 11). Furthermore, no adverse effects were observed in hematology and biochemistry analysis of blood serum both in male and female rat when 0.2 mg/kg and 2 mg/kg of NP-011 was intravenously administrated into rats for 4 weeks daily (FIG. 11). Thus, these results indicate that NP-011 preferentially targets liver after its administration and has excellent safety profiles.

NP-011 shows a powerful anti-fibrotic effect for inhibiting, preventing, and reversing liver fibrosis. NP-011 is superior for clinical application over the full length MFG-E8 (lactadherin) by truncation of the C2 domain for the following reasons: 1) Medin site is eliminated in NP-011 by structural truncation of MFG-E8. Medin is aortic medial amyloid that occurs in people older than 60 years, and accumulated medin is engaged in Alzheimer's disease and type 2 diabetes. MFG-E8 (Lactadherin) has the medin in its component, and it is positioned within C2 domain. Therefore, through the structural truncation of C2 domain from MFG-E8, NP-011 eliminates the concern for inducing amyloid formation in patients upon administration, and the concern for side effects that may cause Alzheimer's disease and diabetes upon repeated administrations into patients. Because the most common liver disease, non-alcoholic fatty liver disease (NAFLD), is closely related with type 2 diabetes, it is a crucial factor for treating liver disease. 2) NP-011 exhibits better binding affinity against collagen by removing the glycosylation sites of MFG-E8. MFG-E8 contains glycosylation sites in the C2 domain. By removing the C2 domain, NP-011 has no glycosylation that might interfere with the binding of the discoidin domain (C1 domain) of MFG-E8 to the accumulated collagen in liver fibrosis. The well-known discoidin domain receptor (DDR) has discoidin domain, stalk region, and transmembrane domain in its structure. Although the mechanism of collagen binding of DDR has not been identified, binding of discoidin domain of DDR to collagen has been reported. Interestingly, the discoidin domain of DDR has no glycosylation (glycosylation sites are only located at stalk region of DDR), therefore, discoidin domain of NP-011 might be similar to the discoidin domain of DDR, and may facilitate binding of NP-011 to accumulated collagen in the liver.

NP-011 has therapeutic efficacy in various liver disease models including nonalcoholic steatohepatitis (NASH). Clinically, nonalcoholic steatohepatitis (NASH) is frequently found in the patient of liver fibrosis because patients with NASH are likely to progress to fibrosis and cirrhosis, and patients with NASH have an almost 10-fold more risk of liver disease-related death than patients with simple steatosis. Thus, the therapeutic capacity of NP-011 in both NASH and fibrosis represent a novel therapy for inhibiting and reversing complex and progressive liver diseases.

High manufacturability is another advantage of NP-011 for clinical application. NP-011 was produced using a yeast system, and this system has several advantages including facile genetic manipulation and rapid growth, as well as eukaryotic features including a secretory pathway for correct protein processing and post-translational modification. Currently, the production yield of NP-011 from yeast resulted 40 mg/L, and this yield provides reliable manufacturing for clinical applications. For example, the production of NP-011 at a general Good Manufacturing Practice (GMP) run scale of 200 L would provide about 3,400 injections for 60-kg adults with the 40 μg/kg of efficacious dosage. Furthermore, NP-011 is produced as a secreted protein (authentic NP-011) without a methionine at the N-terminus of the protein, any tag (e.g., FLAG-tag or his-tag), or random glycosylations, which could induce significant problems upon administration of medications. Thus, in conclusion, NP-011 provides a highly effective and reliable new protein therapy for treating, inhibiting, preventing, and reversing liver fibrosis as well as other liver diseases disclosed herein.

### Example 7: NP-011 Reverses, Inhibits, and Prevents Idiopathic Pulmonary Fibrosis (IPF)

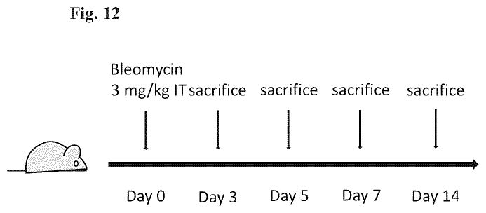

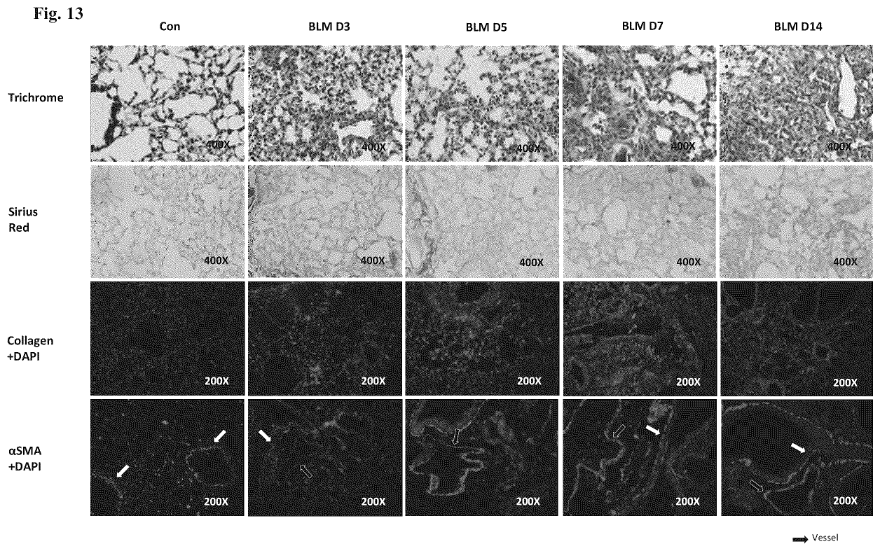

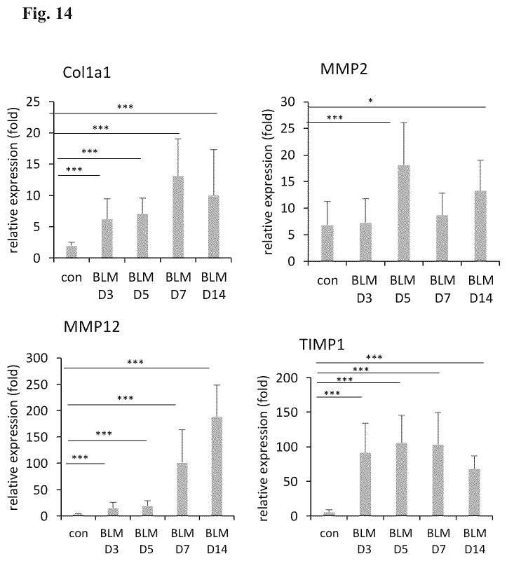

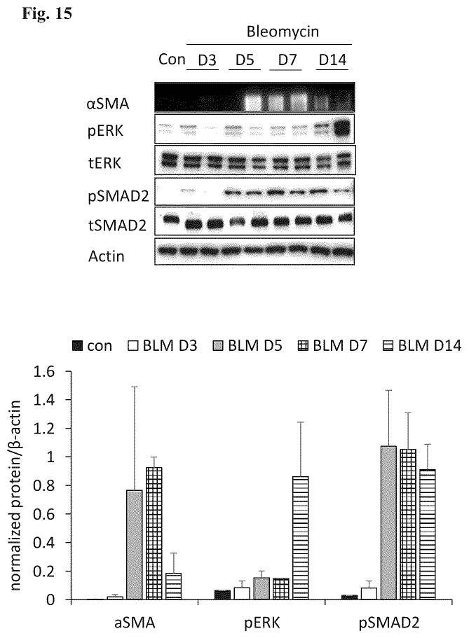

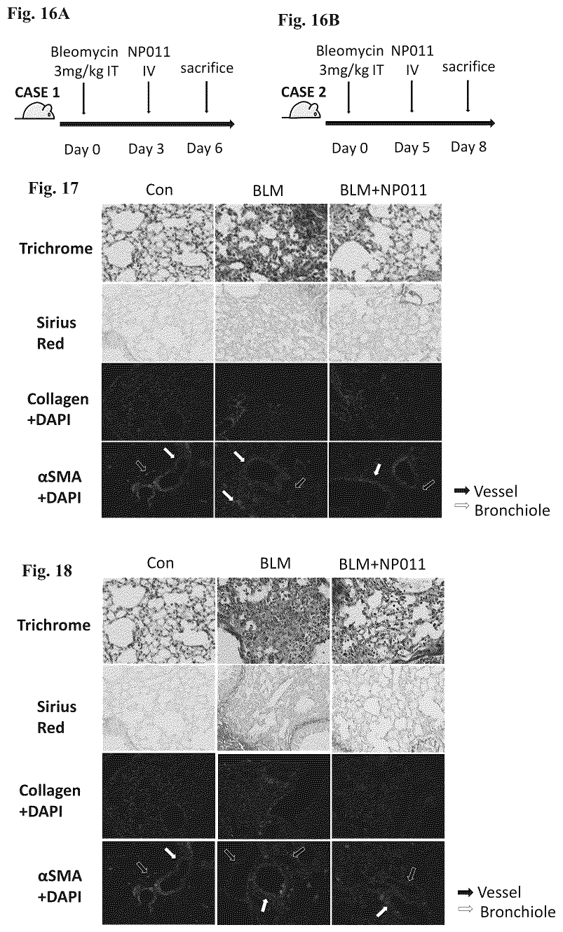

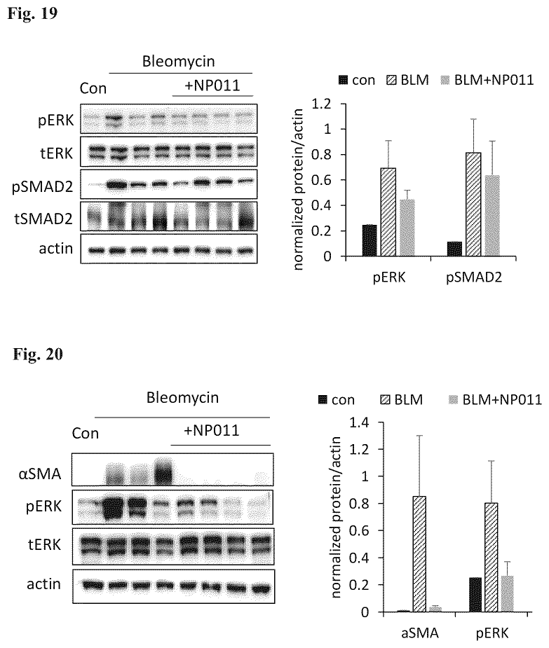

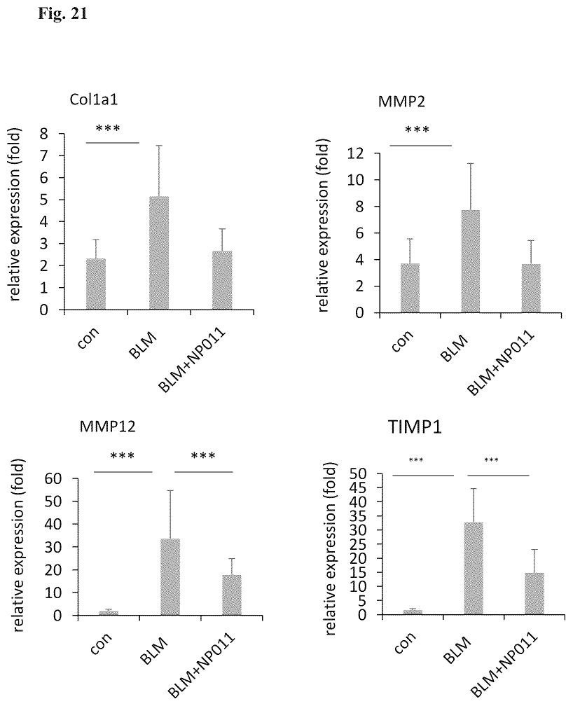

As demonstrated in FIG. 12-FIG. 21, NP-011 is highly efficacious against IPF, and reverses and inhibits IPF in vivo. A model for IPF was induced by injection of bleomycin. Reversal of IPF by NP-011 is evident in histological studies of the lung tissues (FIG. 17 and FIG. 18, as compared to FIG. 13). In addition, NP-011 reverses the bleomycin-induced changes in the expression level of IPF biomarkers such as αSMA, collagen (Colla1), TMP2, MMP2, MMP12, pERK, tERK, pSMAD2 (phosphorylated SMAD2), and tSMAD2 (total SMAD2) (FIG. 12-FIG. 21). Thus, NP-011 provides a highly effective therapy for treating, inhibiting, preventing, and reversing IPF.

### Example 8: NP-011 Reverses, Inhibits, and Prevents Symptoms of Myocardial Infarction and Improves Heart Function

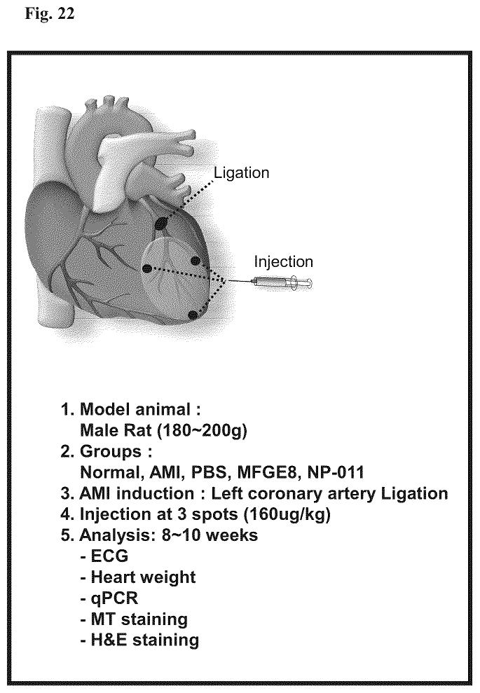

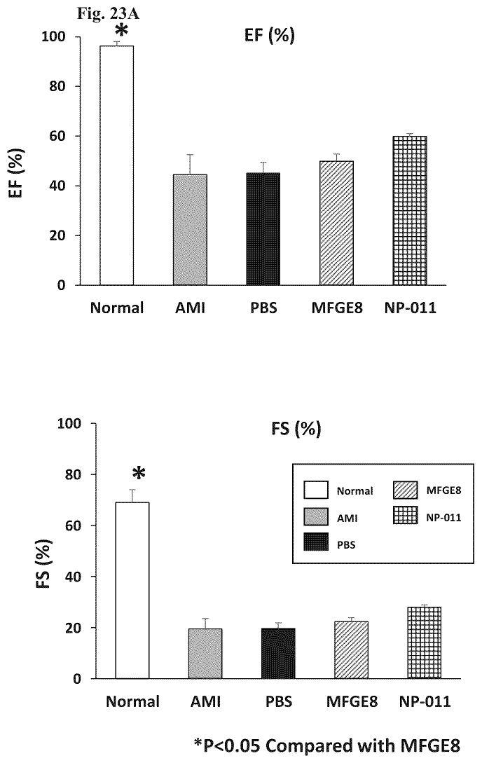

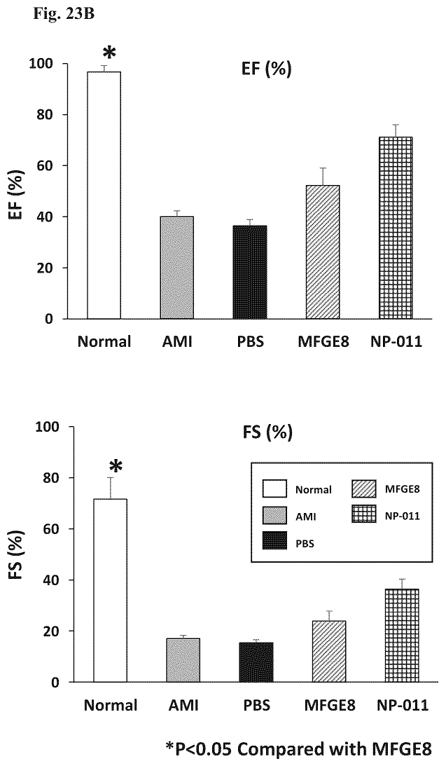

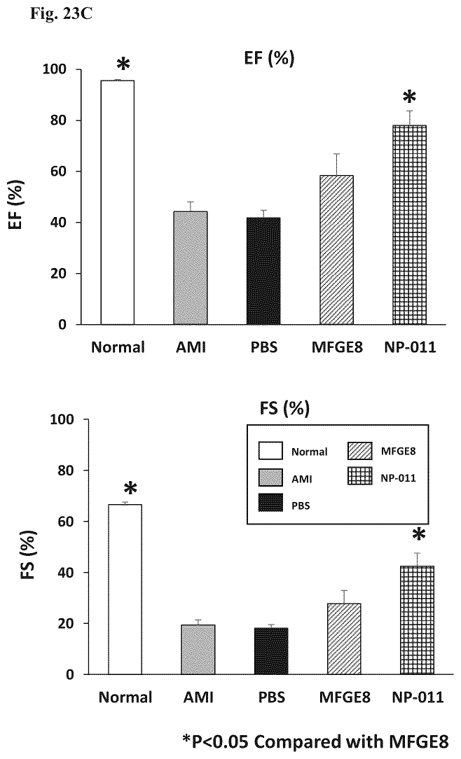

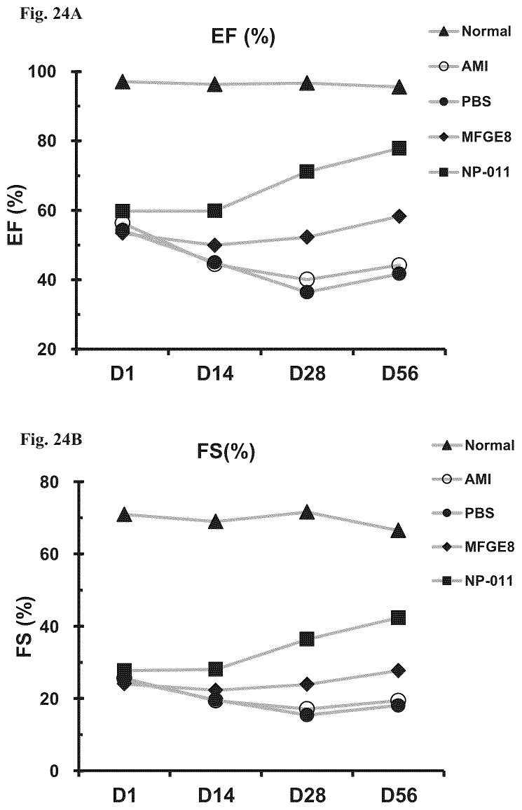

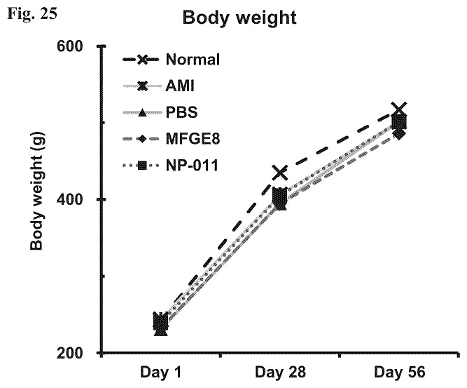

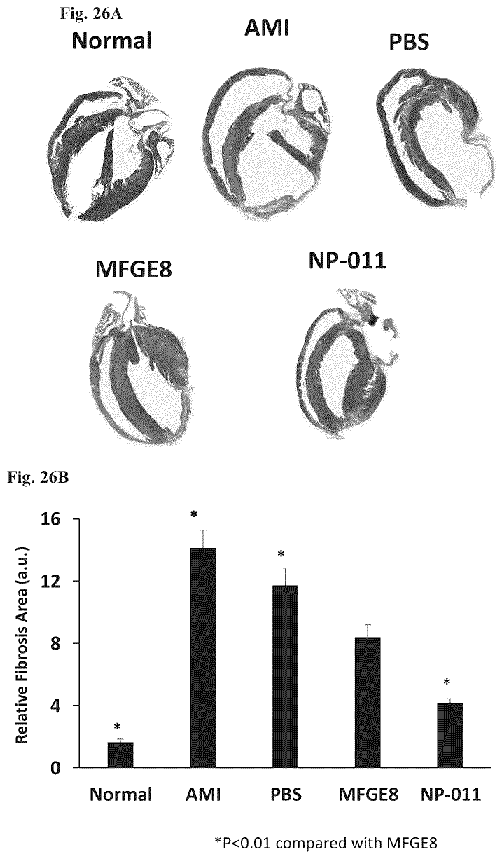

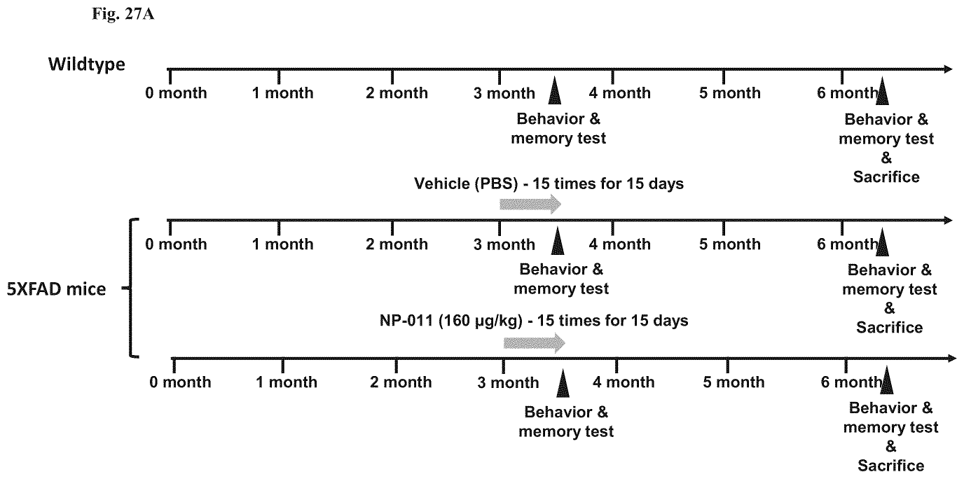

As demonstrated in FIG. 22-FIG. 26, NP-01 1 is highly efficacious against myocardial infarction in vivo. A rat model for myocardial infarction was induced by LAD ligation. Injection of NP-011 increases the heart function as evidenced by increased left ventricular ejection fraction (EF) and fraction shortening (FS) in rats suffering from myocardial infarction (FIG. 23A-24B). Similar body weight for rats injected with NP-011 compared with control rats demonstrate the safety of NP-011 (FIG. 25). Histological studies of cross-sectioned hearts demonstrate that NP-011 also reverses and inhibits the fibrosis associated with myocardial infarction (FIG. 26A-FIG. 26B). Thus, NP-011 provides a highly effective therapy for treating, inhibiting, preventing, and reversing myocardial infarction.

### Example 9: NP-011 Reverses, Inhibits, and Prevents Alzheimer's Disease

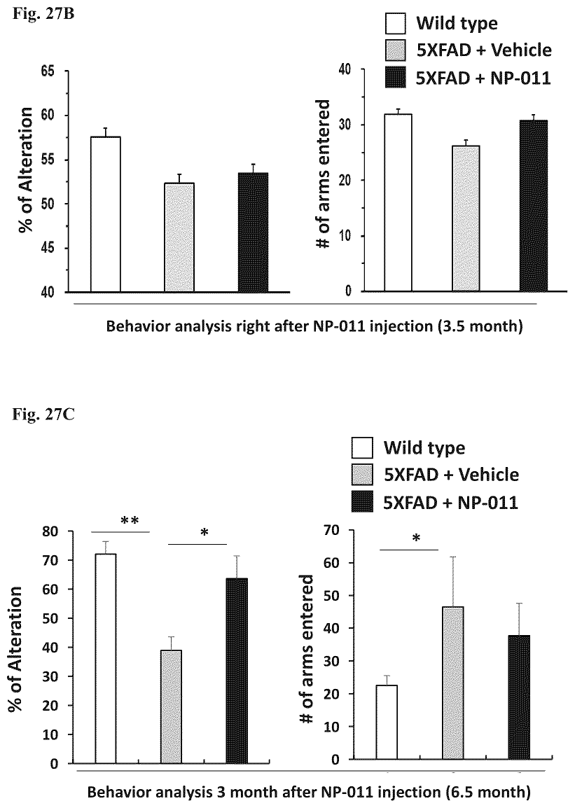

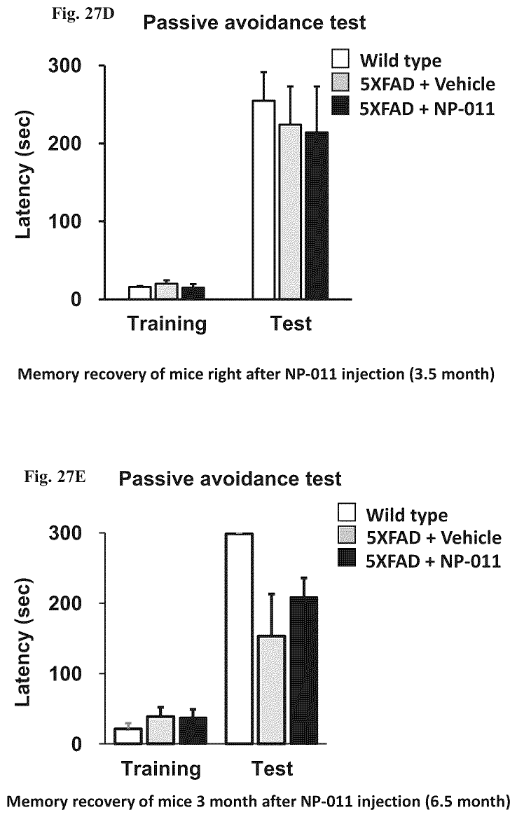

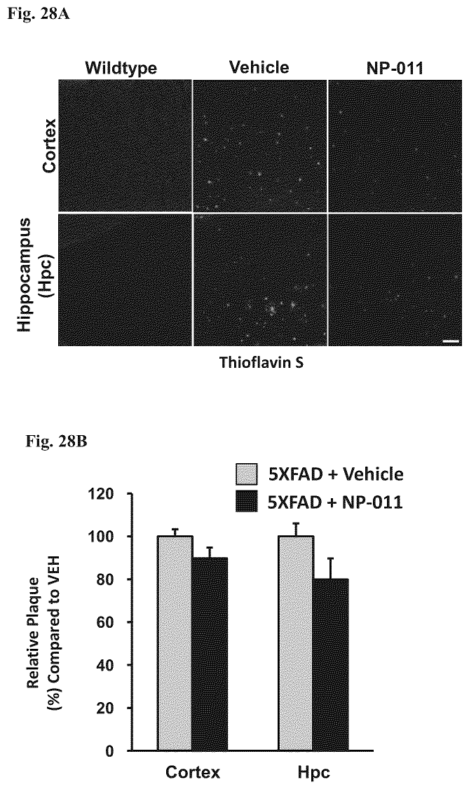

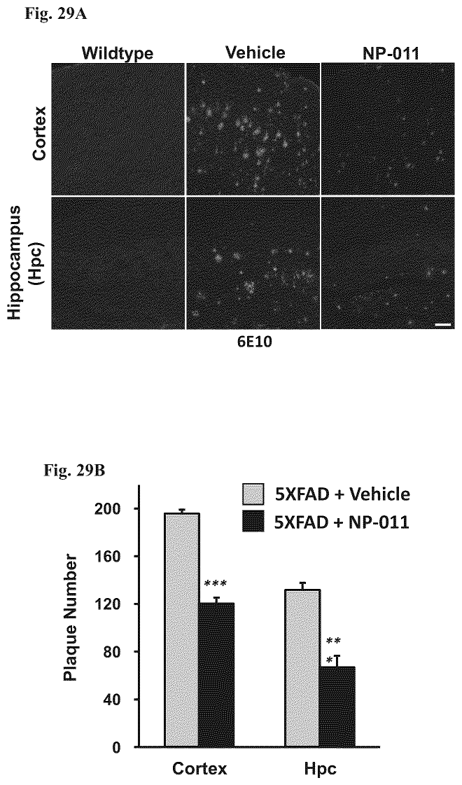

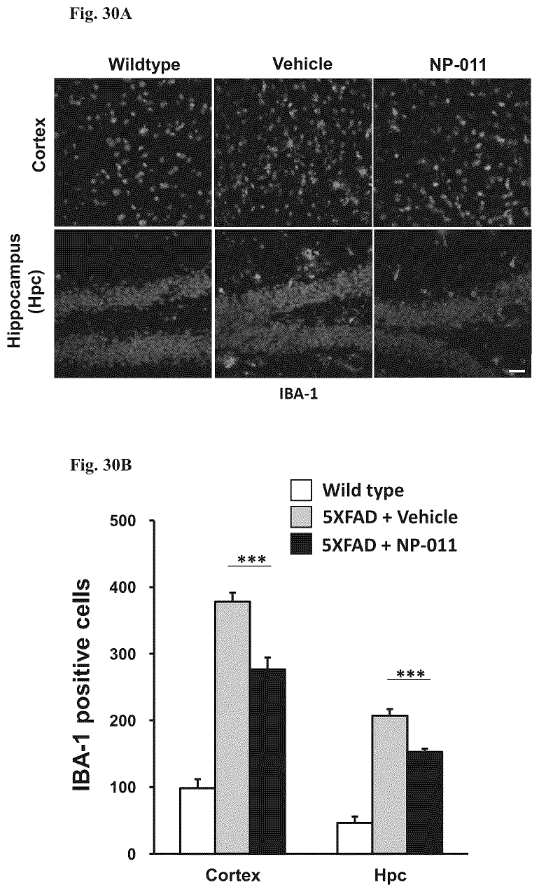

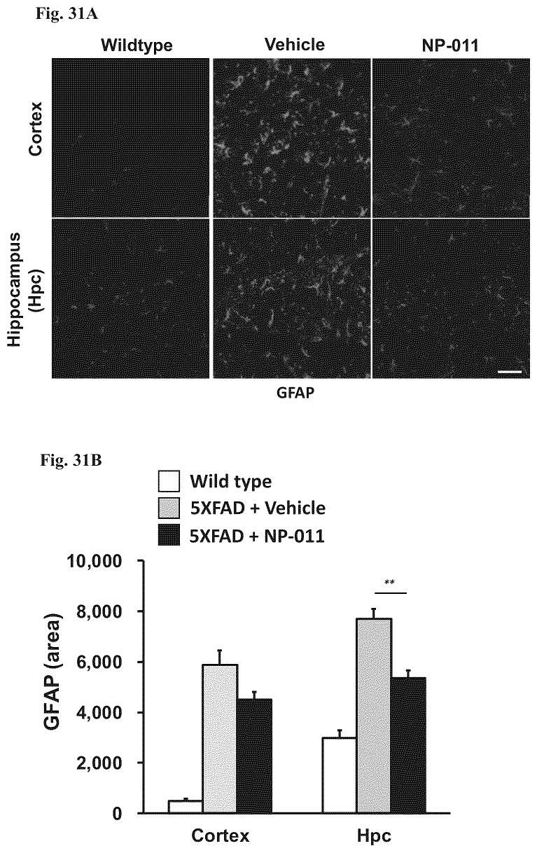

As demonstrated in FIG. 27-FIG. 31, NP-011 is highly efficacious against Alzheimer's Disease in vivo. 5XFAD mice expressing human APP and PSEN1 transgenes with a total of five AD-linked mutations: the Swedish (K670N/M671L), Florida (1716V), and London (V7171) mutations in APP, and the M146L and L286V mutations in PSEN1, were used as a model for Alzheimer's Disease. NP-011 reverses the behaviors and memory loss associated with Alzheimer's Disease (FIG. 27A-FIG. 27E). NP-011 also reverses and inhibits the neuropathological alteration in AD brains. For example, NP-011 specifically decreases the amount of amyloid plaques (FIG. 28A-FIG. 28B) and amyloid beta (FIG. 29A-FIG. 29B). In addition, NP-011 decreases the number of microglial cells, the brain's innate immune system, the presence of which impies neuroinflammation (FIG. 30A-FIG. 30B). Furthermore, NP-011 decreases the amount of glial fibrillary acidic protein (GFAP) that is upregulated in astrocytes of Alzeheimer's Disease (FIG. 31A-FIG. 31B). Accordingly, NP-011 provides a highly effective therapy for treating, inhibiting, preventing, and reversing Alzheimer's Disease.

### REFERENCES

1. Bataller R, Brenner D A. Liver fibrosis. J Clin Invest 2005:115:209-218.
2. Chung W, Jo C, Chung W J, Kim D J. Liver cirrhosis and cancer: comparison of mortality. Hepatol Int 2018:12:269-276.
3. Wynn T A, Ramalingam T R. Mechanisms of fibrosis: therapeutic translation for fibrotic disease. Nat Med 2012:18:1028-1040.
4. Dooley S, ten Dijke P. TGF-β in progression of liver disease. Cell Tissue Res 2012:347:245-256.
5. Gabbiani G. The myofibroblast in wound healing and fibrocontractive diseases. J Pathol 2003:200:500-503.
6. Kalluri R, Zeisberg M. Fibroblasts in cancer. Nat Rev Cancer 2006:6:392-401.
7. Troeger J S, Mederacke I, Gwak G Y, Dapito D H, Mu X, Hsu C C, Pradere J P, Friedman R A, Schwabe R F. Deactivation of hepatic stellate cells during liver fibrosis resolution in mice. Gastroenterology 2012:143:1073-1083.
8. Uchiyama A, Yamada K, Ogino S, Yokoyama Y, Takeuchi Y, Udey M C, Ishikawa O, Motegi S. MFG-E8 regulates angiogenesis in cutaneous wound healing. Am J Pathol 2014:184:1981-1990.
9. Yoshida H, Kawane K, Koike M, Mori Y, Uchiyama Y, Nagata S. Phosphatidylserine-dependent engulfment by macrophages of nuclei from erythroid precursor cells. Nature 2005:437:754-758.
10. Hanayama R, Tanaka M, Miwa K, Shinohara A, Iwamatsu A, Nagata S. Identification of a factor that links apoptotic cells to phagocytes. Nature 2002:417:182-187.
11. Yi Y S. Functional Role of Milk Fat Globule-Epidermal Growth Factor VIII in Macrophage-Mediated Inflammatory Responses and Inflammatory/Autoimmune Diseases. Mediators Inflamm 2016:2016:5628486.
12. An S Y, Jang Y J, Lim H J, Han J, Lee J, Lee G, Park J Y, Park S Y, Kim J H, Do B R, Han C, Park H K, Kim O H, Song M J, Kim S J, Kim J H. Milk Fat Globule-EGF Factor 8, Secreted by Mesenchymal Stem Cells, Protects Against Liver Fibrosis in Mice. Gastroenterology 2017:152:1174-1186.
13. Atabai K, Jame S, Azhar N, Kuo A, Lam M, Mckleroy W, Dehart G, Rahman S, Xia D D, Melton A C, Wolters P, Emson C L, Turner S M, Werb Z, Sheppard D. Mfge8 diminishes the severity of tissue fibrosis in mice by binding and targeting collagen for uptake by macrophages. J Clin Invest 2009:119:3713-3722.
14. Ye H, Li B, Subramanian V, Choi B H, Liang Y, Harikishore A, Chakraborty G, Baek K, Yoon H S. NMR solution structure of C2 domain of MFG-E8 and insights into its molecular recognition with phosphatidylserine. Biochim Biophys Acta 2013:1828: 1083-1093.
15. Köhler C, Bell A W, Bowen W C, Monga S P, Fleig W, Michalopoulos G K. Expression of Notch-1 and its ligand Jagged-1 in rat liver during liver regeneration. Hepatology 2004:39:1056-1065.
16. Ahmad R, Ahmed S, Khan N U, Hasnain A U. Operculina turpethum attenuates N-nitrosodimethylamine induced toxic liver injury and clastogenicity in rats. Chem Biol Interact 2009:181:145-153.
17. Sarrazy V, Koehler A, Chow M L, Zimina E, Li C X, Kato H, Caldarone C A, Hinz B. Integrins αvβ5 and αvβ3 promote latent TGF-β1 activation by human cardiac fibroblast contraction. Cardiovasc Res 2014:102:407-417.
18. Théret N, Musso O, L'Helgoualc'h A, Clément B. Activation of matrix metalloproteinase-2from hepatic stellate cells requires interactions with hepatocytes. Am J Pathol 1997:150:51-58.
19. Iredale J P, Benyon R C, Arthur M J, Ferris W F, Alcolado R, Winwood P J, Clark N, Murphy G. Tissue inhibitor of metalloproteinase-1 messenger RNA expression is enhanced relative to interstitial collagenase messenger RNA in experimental liver injury and fibrosis. Hepatology 1996:24:176-184.
20. Liu B, Shi D, Chang S, Gong X, Yu Y, Sun Z, Wu J. Characterization and immunological activity of different forms of recombinant secreted Hc of botulinum neurotoxin serotype B products expressed in yeast. Sci Rep 2015:5:7678.
21. Westermark P, Johnson K H, O'Brien T D, Betsholtz C. Islet amyloid polypeptide—a novel controversy in diabetes research. Diabetologia 1992:35:297-303.
22. Westermark P, Mucchiano G, Marthin T, Johnson K H, Sletten K. Apolipoprotein A1-derived amyloid in human aortic atherosclerotic plaques. Am J Pathol 1995;147:1186-1192.
23. Häggqvist B, Näslund J, Sletten K, Westermark G T, Mucchiano G, Tjernberg L O, Nordstedt C, Engström U, Westermark P. Medin: an integral fragment of aortic smooth muscle cell-produced lactadherin forms the most common human amyloid. Proc Natl Acad Sci U S A 1999;96:8669-8674.
24. Ong J P, Younossi Z M. Epidemiology and natural history of NAFLD and NASH. Clin Liver Dis 2007:11:1-16, vii.
25. Assy N, Kaita K, Mymin D, Levy C, Rosser B, Minuk G. Fatty infiltration of liver in hyperlipidemic patients. Dig Dis Sci 2000:45:1929-1934.
26. Dorison A, Dussaule JC, Chatziantoniou C. The Role of Discoidin Domain Receptor 1 in Inflammation, Fibrosis and Renal Disease. Nephron 2017:137:212-220.
27. Borza C M, Pozzi A. Discoidin domain receptors in disease. Matrix Biol 2014;34:185-192.
28. Anstee Q M, Targher G, Day C P. Progression of NAFLD to diabetes mellitus, cardiovascular disease or cirrhosis. Nat Rev Gastroenterol Hepatol 2013;10:330-344.
29. Sola R J, Griebenow K. Effects of glycosylation on the stability of protein pharmaceuticals. J Pharm Sci 2009;98:1223-1245.

1-33. (canceled)

34. A pharmaceutical composition comprising an engineered polypeptide, wherein the engineered polypeptide comprises
a) an epidermal growth factor (EGF)-like domain of an MFG-E8 polypeptide; and
b) a C1 domain of an MFG-E8 polypeptide,
wherein the engineered polypeptide lacks at least 1, 2, 3, 4, 5, 10, 15, 20, 25, 30, 35, 40, 45, 50, 55, 60, 65, 70, 75, 80, 85, 90, 95, 100, or 105 amino acids within amino acids 226-335 of the MFG-E8 polypeptide.

35. The pharmaceutical composition of claim 34, wherein the engineered polypeptide lacks at least 1, 2, 3, 4, 5, 10, 15, 20, 25, 30, 35, 40, 45, 50, 55, 60, 65, 70, 75, 80, 85, 90, 95, 100, or 105 amino acids within amino acids 226-335 of SEQ ID NO: 10 or SEQ ID NO: 12.

36. The pharmaceutical composition of claim 34, wherein the engineered polypeptide lacks at least 105 amino acids within the amino acids 226-335 of SEQ ID NO: 10 or SEQ ID NO: 12.

37. The pharmaceutical composition of claim 34, wherein the engineered polypeptide lacks amino acid residues 226-387 of SEQ ID NO: 10 or SEQ ID NO: 12.

38. The pharmaceutical composition of claim 34, wherein a sequence of the EGF-like domain or a sequence of the C1 domain is from SEQ ID NO: 10 or SEQ ID NO: 12.

39. The pharmaceutical composition of claim 34, wherein the engineered polypeptide comprises at least 180, 190, 200, 210, 215, 220, 225, 230, 235, 240, 245, 250, 255, 260, 265, 270, 275, or 280 amino acids of the MFG-E8 polypeptide.

40. The pharmaceutical composition of claim 34, wherein the engineered polypeptide further comprises a heterologous sequence.

41. The pharmaceutical composition of claim 40, wherein the heterologous sequence increases the half life of the polypeptide in vivo.

42. The pharmaceutical composition of claim 40, wherein the heterologous sequence comprises an Fc portion of an immunoglobulin.

43. The pharmaceutical composition of claim 40, wherein the heterologous sequence comprises a FLAG tag, a HIS tag, and/or GST.

44. The pharmaceutical composition of claim 34, wherein the engineered polypeptide is capable of increasing collagen or apoptotic cell uptake by macrophages.

45. The pharmaceutical composition of claim 34, wherein the pharmaceutical composition comprises one or more pharmaceutically acceptable carriers and/or diluents.

46. A method of treating or preventing a disorder in a subject in need thereof, the method comprising administering to the subject the pharmaceutical composition of claim 34.

47. The method of claim 46, wherein the disorder is chronic or acute fibrosis, cirrhosis, steatosis, nonalcoholic steatohepatitis, lung fibrosis, idiopathic pulmonary fibrosis, myocardial infarction, kidney disease, or Alzheimer's Disease.

48. The method of claim 47, wherein the disorder is Alzheimer's Disease, and the subject has (i) at least one mutation in amyloid precursor protein (APP) selected from K670N/M671L, I716V, and V717I; and/or (ii) at least one mutation in PSEN1 selected from M146L and L286V.

49. A method of reducing inflammation in a subject in need thereof, the method comprising administering to the subject the pharmaceutical composition of claim 34.

50. The method of claim 46, wherein the subject is a human.

51. The method of claim 49, wherein the subject is a human.


SEQUENCE LISTING (41 sequences)

SEQ ID NO: 1 - DNA, 1780 bp, Homo sapiens
  source 1..1780: mol_type = genomic DNA
agaaccccgcggggtctgagcagcccagcgtgcccattccagcgcccgcgtccccgcagcatgccgcgcccccgcctgctggccgcgctgtgcggcgcgctgctctgcgcccccagcctcctcgtcgccctggatatctgttccaaaaacccctgccacaacggtggtttatgcgaggagatttcccaagaagtgcgaggagatgtcttcccctcgtacacctgcacgtgccttaagggctacgcgggcaaccactgtgagacgaaatgtgtcgagccactgggcctggagaatgggaacattgccaactcacagatcgccgcctcgtctgtgcgtgtgaccttcttgggtttgcagcattgggtcccggagctggcccgcctgaaccgcgcaggcatggtcaatgcctggacacccagcagcaatgacgataacccctggatccaggtgaacctgctgcggaggatgtgggtaacaggtgtggtgacgcagggtgccagccgcttggccagtcatgagtacctgaaggccttcaaggtggcctacagccttaatggacacgaattcgatttcatccatgatgttaataaaaaacacaaggagtttgtgggtaactggaacaaaaacgcggtgcatgtcaacctgtttgagacccctgtggaggctcagtacgtgagattgtaccccacgagctgccacacggcctgcactctgcgctttgagctactgggctgtgagctgaacggatgcgccaatcccctgggcctgaagaataacagcatccctgacaagcagatcacggcctccagcagctacaagacctggggcttgcatctcttcagctggaacccctcctatgcacggctggacaagcagggcaacttcaacgcctgggttgcggggagctacggtaacgatcagtggctgcagatcttccctggcaactgggacaaccactcccacaagaagaacttgtttgagacgcccatcctggctcgctatgtgcgcatcctgcctgtagcctggcacaaccgcatcgccctgcgcctggagctgctgggctgttagtggccacctgccacccccaggtcttcctgctttccatgggcccgctgcctcttggcttctcagcccctttaaatcaccatagggctggggactggggaaggggagggtgttcagaggcagcaccaccacacagtcacccctccctccctctttcccaccctccacctctcacgggccctgccccagcccctaagccccgtcccctaacccccagtcctcactgtcctgttttcttaggcactgagggatctgagtaggtctgggatggacaggaaagggcaaagtagggcgtgtggtttccctgcccctgtccggaccgccgatcccaggtgcgtgtgtctctgtctctcctagcccctctctcacacatcacattcccatggtggcctcaagaaaggcccggaagcgccaggctggagataacagcctcttgcccgtcggccctgcgtcggccctggggtaccatgtggccacaactgctgtggccccctgtccccaagacacttccccttgtctccctggttgcctctcttgccccttgtcctgaagcccagcgacacagaagggggtggggcgggtctatggggagaaagggagcgaggtcagaggagggcatgggttggcagggtgggcgtttggggccctctatgctggcttttcaccccagaggacacaggcagcttccaaaatatatttatcttcttcacgggaa

SEQ ID NO: 2 - protein, 335 aa, Homo sapiens
MPRPRLLAALCGALLCAPSLLVALDICSKNPCHNGGLCEEISQEVRGDVFPSYTCTCLKGYAGNHCETKCVEPLGLENGNIANSQIAASSVRVTFLGLQHWVPELARLNRAGMVNAWTPSSNDDNPWIQVNLLRRMWVTGVVTQGASRLASHEYLKAFKVAYSLNGHEFDFIHDVNKKHKEFVGNWNKNAVHVNLFETPVEAQYVRLYPTSCHTACTLRFELLGCELNGCANPLGLKNNSIPDKQITASSSYKTWGLHLFSWNPSYARLDKQGNFNAWVAGSYGNDQWLQIFPGNWDNHSHKKNLFETPILARYVRILPVAWHNRIALRLELLGC

SEQ ID NO: 3 - DNA, 1804 bp, Homo sapiens
  source 1..1804: mol_type = genomic DNA
agaaccccgcggggtctgagcagcccagcgtgcccattccagcgcccgcgtccccgcagcatgccgcgcccccgcctgctggccgcgctgtgcggcgcgctgctctgcgcccccagcctcctcgtcgccctggaatgtgtcgagccactgggcctggagaatgggaacattgccaactcacagatcgccgcctcgtctgtgcgtgtgaccttcttgggtttgcagcattgggtcccggagctggcccgcctgaaccgcgcaggcatggtcaatgcctggacacccagcagcaatgacgataacccctggatccaggtgaacctgctgcggaggatgtgggtaacaggtgtggtgacgcagggtgccagccgcttggccagtcatgagtacctgaaggccttcaaggtggcctacagccttaatggacacgaattcgatttcatccatgatgttaataaaaaacacaaggagtttgtgggtaactggaacaaaaacgcggtgcatgtcaacctgtttgagacccctgtggaggctcagtacgtgagattgtaccccacgagctgccacacggcctgcactctgcgctttgagctactgggctgtgagctgaacggatgcgccaatcccctgggcctgaagaataacagcatccctgacaagcagatcacggcctccagcagctacaagacctggggcttgcatctcttcagctggaacccctcctatgcacggctggacaagcagggcaacttcaacgcctgggttgcggggagctacggtaacgatcagtggctgcaggtggacctgggctcctcgaaggaggtgacaggcatcatcacccagggggcccgtaactttggctctgtccagtttgtggcatcctacaaggttgcctacagtaatgacagtgcgaactggactgagtaccaggaccccaggactggcagcagtaagatcttccctggcaactgggacaaccactcccacaagaagaacttgtttgagacgcccatcctggctcgctatgtgcgcatcctgcctgtagcctggcacaaccgcatcgccctgcgcctggagctgctgggctgttagtggccacctgccacccccaggtcttcctgctttccatgggcccgctgcctcttggcttctcagcccctttaaatcaccatagggctggggactggggaaggggagggtgttcagaggcagcaccaccacacagtcacccctccctccctctttcccaccctccacctctcacgggccctgccccagcccctaagccccgtcccctaacccccagtcctcactgtcctgttttcttaggcactgagggatctgagtaggtctgggatggacaggaaagggcaaagtagggcgtgtggtttccctgcccctgtccggaccgccgatcccaggtgcgtgtgtctctgtctctcctagcccctctctcacacatcacattcccatggtggcctcaagaaaggcccggaagcgccaggctggagataacagcctcttgcccgtcggccctgcgtcggccctggggtaccatgtggccacaactgctgtggccccctgtccccaagacacttccccttgtctccctggttgcctctcttgccccttgtcctgaagcccagcgacacagaagggggtggggcgggtctatggggagaaagggagcgaggtcagaggagggcatgggttggcagggtgggcgtttggggccctctatgctggcttttcaccccagaggacacaggcagcttccaaaatatatttatcttcttcacgggaa

SEQ ID NO: 4 - protein, 343 aa, Homo sapiens
MPRPRLLAALCGALLCAPSLLVALECVEPLGLENGNIANSQIAASSVRVTFLGLQHWVPELARLNRAGMVNAWTPSSNDDNPWIQVNLLRRMWVTGVVTQGASRLASHEYLKAFKVAYSLNGHEFDFIHDVNKKHKEFVGNWNKNAVHVNLFETPVEAQYVRLYPTSCHTACTLRFELLGCELNGCANPLGLKNNSIPDKQITASSSYKTWGLHLFSWNPSYARLDKQGNFNAWVAGSYGNDQWLQVDLGSSKEVTGIITQGARNFGSVQFVASYKVAYSNDSANWTEYQDPRTGSSKIFPGNWDNHSHKKNLFETPILARYVRILPVAWHNRIALRLELLGC

SEQ ID NO: 5 - DNA, 1989 bp, Homo sapiens
  source 1..1989: mol_type = genomic DNA
agaaccccgcggggtctgagcagcccagcgtgcccattccagcgcccgcgtccccgcagcatgccgcgcccccgcctgctggccgcgctgtgcggcgcgctgctctgcgcccccagcctcctcgtcgccctggggtgatgtggccttttccagaaggaggaaacaccatacctatcttacacacagatatctgttccaaaaacccctgccacaacggtggtttatgcgaggagatttcccaagaagtgcgaggagatgtcttcccctcgtacacctgcacgtgccttaagggctacgcgggcaaccactgtgagacgaaatgtgtcgagccactgggcctggagaatgggaacattgccaactcacagatcgccgcctcgtctgtgcgtgtgaccttcttgggtttgcagcattgggtcccggagctggcccgcctgaaccgcgcaggcatggtcaatgcctggacacccagcagcaatgacgataacccctggatccaggtgaacctgctgcggaggatgtgggtaacaggtgtggtgacgcagggtgccagccgcttggccagtcatgagtacctgaaggccttcaaggtggcctacagccttaatggacacgaattcgatttcatccatgatgttaataaaaaacacaaggagtttgtgggtaactggaacaaaaacgcggtgcatgtcaacctgtttgagacccctgtggaggctcagtacgtgagattgtaccccacgagctgccacacggcctgcactctgcgctttgagctactgggctgtgagctgaacggatgcgccaatcccctgggcctgaagaataacagcatccctgacaagcagatcacggcctccagcagctacaagacctggggcttgcatctcttcagctggaacccctcctatgcacggctggacaagcagggcaacttcaacgcctgggttgcggggagctacggtaacgatcagtggctgcaggtggacctgggctcctcgaaggaggtgacaggcatcatcacccagggggcccgtaactttggctctgtccagtttgtggcatcctacaaggttgcctacagtaatgacagtgcgaactggactgagtaccaggaccccaggactggcagcagtaagatcttccctggcaactgggacaaccactcccacaagaagaacttgtttgagacgcccatcctggctcgctatgtgcgcatcctgcctgtagcctggcacaaccgcatcgccctgcgcctggagctgctgggctgttagtggccacctgccacccccaggtcttcctgctttccatgggcccgctgcctcttggcttctcagcccctttaaatcaccatagggctggggactggggaaggggagggtgttcagaggcagcaccaccacacagtcacccctccctccctctttcccaccctccacctctcacgggccctgccccagcccctaagccccgtcccctaacccccagtcctcactgtcctgttttcttaggcactgagggatctgagtaggtctgggatggacaggaaagggcaaagtagggcgtgtggtttccctgcccctgtccggaccgccgatcccaggtgcgtgtgtctctgtctctcctagcccctctctcacacatcacattcccatggtggcctcaagaaaggcccggaagcgccaggctggagataacagcctcttgcccgtcggccctgcgtcggccctggggtaccatgtggccacaactgctgtggccccctgtccccaagacacttccccttgtctccctggttgcctctcttgccccttgtcctgaagcccagcgacacagaagggggtggggcgggtctatggggagaaagggagcgaggtcagaggagggcatgggttggcagggtgggcgtttggggccctctatgctggcttttcaccccagaggacacaggcagcttccaaaatatatttatcttcttcacgggaa

SEQ ID NO: 6 - protein, 379 aa, Homo sapiens
MWPFPEGGNTIPILHTDICSKNPCHNGGLCEEISQEVRGDVFPSYTCTCLKGYAGNHCETKCVEPLGLENGNIANSQIAASSVRVTFLGLQHWVPELARLNRAGMVNAWTPSSNDDNPWIQVNLLRRMWVTGVVTQGASRLASHEYLKAFKVAYSLNGHEFDFIHDVNKKHKEFVGNWNKNAVHVNLFETPVEAQYVRLYPTSCHTACTLRFELLGCELNGCANPLGLKNNSIPDKQITASSSYKTWGLHLFSWNPSYARLDKQGNFNAWVAGSYGNDQWLQVDLGSSKEVTGIITQGARNFGSVQFVASYKVAYSNDSANWTEYQDPRTGSSKIFPGNWDNHSHKKNLFETPILARYVRILPVAWHNRIALRLELLGC

SEQ ID NO: 7 - DNA, 2544 bp, Homo sapiens
  source 1..2544: mol_type = genomic DNA
acctccactgttgacaaacttagacaaagccccggggaccgggctgggcagaggggcggcttcttccgctgcgccctggcgggacagggggatgcggccctgctgtctctgcgctggggcttttgggctgggactcgggacatcgggtgacagccctgccgcccccagggatgcggcttacagataatgacaaaggaatccgctgtgtcgggcctctcttttccctggtgaaaaatgaggccagggaactgcgtttgactttcgaaccccttccacctgggagattctaggactctagtatggataagtcttgtctggataactttgtcctggccatctccctgccaactccagttggctggacagttcattggatttttgcgctcccaattgtccgtgcctggtcacataagggaagggccggggagtcggtgcaatggacgcaggccgtaagtggggcccgggaggggacccagaggcttcgaggagcttggaagagggctgcctgctgatgggagtctcctgactccctccctcccgcggccttggccggctgctgtatcttccccggtcctcctccgcctcccaggaggcctccggaggccagctgggccccttgcaggctggacttgcggatgccccgtgccattcaccgtggagcgctgggagggagtcagggccaggactctttaggtggcccctccatcattttctcatagaaatgggattgactgaagcaagatatctgttccaaaaacccctgccacaacggtggtttatgcgaggagatttcccaagaagtgcgaggagatgtcttcccctcgtacacctgcacgtgccttaagggctacgcgggcaaccactgtgagacgaaatgtgtcgagccactgggcctggagaatgggaacattgccaactcacagatcgccgcctcgtctgtgcgtgtgaccttcttgggtttgcagcattgggtcccggagctggcccgcctgaaccgcgcaggcatggtcaatgcctggacacccagcagcaatgacgataacccctggatccaggtgaacctgctgcggaggatgtgggtaacaggtgtggtgacgcagggtgccagccgcttggccagtcatgagtacctgaaggccttcaaggtggcctacagccttaatggacacgaattcgatttcatccatgatgttaataaaaaacacaaggagtttgtgggtaactggaacaaaaacgcggtgcatgtcaacctgtttgagacccctgtggaggctcagtacgtgagattgtaccccacgagctgccacacggcctgcactctgcgctttgagctactgggctgtgagctgaacggatgcgccaatcccctgggcctgaagaataacagcatccctgacaagcagatcacggcctccagcagctacaagacctggggcttgcatctcttcagctggaacccctcctatgcacggctggacaagcagggcaacttcaacgcctgggttgcggggagctacggtaacgatcagtggctgcaggtggacctgggctcctcgaaggaggtgacaggcatcatcacccagggggcccgtaactttggctctgtccagtttgtggcatcctacaaggttgcctacagtaatgacagtgcgaactggactgagtaccaggaccccaggactggcagcagtaagatcttccctggcaactgggacaaccactcccacaagaagaacttgtttgagacgcccatcctggctcgctatgtgcgcatcctgcctgtagcctggcacaaccgcatcgccctgcgcctggagctgctgggctgttagtggccacctgccacccccaggtcttcctgctttccatgggcccgctgcctcttggcttctcagcccctttaaatcaccatagggctggggactggggaaggggagggtgttcagaggcagcaccaccacacagtcacccctccctccctctttcccaccctccacctctcacgggccctgccccagcccctaagccccgtcccctaacccccagtcctcactgtcctgttttcttaggcactgagggatctgagtaggtctgggatggacaggaaagggcaaagtagggcgtgtggtttccctgcccctgtccggaccgccgatcccaggtgcgtgtgtctctgtctctcctagcccctctctcacacatcacattcccatggtggcctcaagaaaggcccggaagcgccaggctggagataacagcctcttgcccgtcggccctgcgtcggccctggggtaccatgtggccacaactgctgtggccccctgtccccaagacacttccccttgtctccctggttgcctctcttgccccttgtcctgaagcccagcgacacagaagggggtggggcgggtctatggggagaaagggagcgaggtcagaggagggcatgggttggcagggtgggcgtttggggccctctatgctggcttttcaccccagaggacacaggcagcttccaaaatatatttatcttcttcacgggaa

SEQ ID NO: 8 - protein, 275 aa, Homo sapiens
MVNAWTPSSNDDNPWIQVNLLRRMWVTGVVTQGASRLASHEYLKAFKVAYSLNGHEFDFIHDVNKKHKEFVGNWNKNAVHVNLFETPVEAQYVRLYPTSCHTACTLRFELLGCELNGCANPLGLKNNSIPDKQITASSSYKTWGLHLFSWNPSYARLDKQGNFNAWVAGSYGNDQWLQVDLGSSKEVTGIITQGARNFGSVQFVASYKVAYSNDSANWTEYQDPRTGSSKIFPGNWDNHSHKKNLFETPILARYVRILPVAWHNRIALRLELLGC

SEQ ID NO: 9 - DNA, 1936 bp, Homo sapiens
  source 1..1936: mol_type = genomic DNA
agaaccccgcggggtctgagcagcccagcgtgcccattccagcgcccgcgtccccgcagcatgccgcgcccccgcctgctggccgcgctgtgcggcgcgctgctctgcgcccccagcctcctcgtcgccctggatatctgttccaaaaacccctgccacaacggtggtttatgcgaggagatttcccaagaagtgcgaggagatgtcttcccctcgtacacctgcacgtgccttaagggctacgcgggcaaccactgtgagacgaaatgtgtcgagccactgggcctggagaatgggaacattgccaactcacagatcgccgcctcgtctgtgcgtgtgaccttcttgggtttgcagcattgggtcccggagctggcccgcctgaaccgcgcaggcatggtcaatgcctggacacccagcagcaatgacgataacccctggatccaggtgaacctgctgcggaggatgtgggtaacaggtgtggtgacgcagggtgccagccgcttggccagtcatgagtacctgaaggccttcaaggtggcctacagccttaatggacacgaattcgatttcatccatgatgttaataaaaaacacaaggagtttgtgggtaactggaacaaaaacgcggtgcatgtcaacctgtttgagacccctgtggaggctcagtacgtgagattgtaccccacgagctgccacacggcctgcactctgcgctttgagctactgggctgtgagctgaacggatgcgccaatcccctgggcctgaagaataacagcatccctgacaagcagatcacggcctccagcagctacaagacctggggcttgcatctcttcagctggaacccctcctatgcacggctggacaagcagggcaacttcaacgcctgggttgcggggagctacggtaacgatcagtggctgcaggtggacctgggctcctcgaaggaggtgacaggcatcatcacccagggggcccgtaactttggctctgtccagtttgtggcatcctacaaggttgcctacagtaatgacagtgcgaactggactgagtaccaggaccccaggactggcagcagtaagatcttccctggcaactgggacaaccactcccacaagaagaacttgtttgagacgcccatcctggctcgctatgtgcgcatcctgcctgtagcctggcacaaccgcatcgccctgcgcctggagctgctgggctgttagtggccacctgccacccccaggtcttcctgctttccatgggcccgctgcctcttggcttctcagcccctttaaatcaccatagggctggggactggggaaggggagggtgttcagaggcagcaccaccacacagtcacccctccctccctctttcccaccctccacctctcacgggccctgccccagcccctaagccccgtcccctaacccccagtcctcactgtcctgttttcttaggcactgagggatctgagtaggtctgggatggacaggaaagggcaaagtagggcgtgtggtttccctgcccctgtccggaccgccgatcccaggtgcgtgtgtctctgtctctcctagcccctctctcacacatcacattcccatggtggcctcaagaaaggcccggaagcgccaggctggagataacagcctcttgcccgtcggccctgcgtcggccctggggtaccatgtggccacaactgctgtggccccctgtccccaagacacttccccttgtctccctggttgcctctcttgccccttgtcctgaagcccagcgacacagaagggggtggggcgggtctatggggagaaagggagcgaggtcagaggagggcatgggttggcagggtgggcgtttggggccctctatgctggcttttcaccccagaggacacaggcagcttccaaaatatatttatcttcttcacgggaa

SEQ ID NO: 10 - protein, 387 aa, Homo sapiens
MPRPRLLAALCGALLCAPSLLVALDICSKNPCHNGGLCEEISQEVRGDVFPSYTCTCLKGYAGNHCETKCVEPLGLENGNIANSQIAASSVRVTFLGLQHWVPELARLNRAGMVNAWTPSSNDDNPWIQVNLLRRMWVTGVVTQGASRLASHEYLKAFKVAYSLNGHEFDFIHDVNKKHKEFVGNWNKNAVHVNLFETPVEAQYVRLYPTSCHTACTLRFELLGCELNGCANPLGLKNNSIPDKQITASSSYKTWGLHLFSWNPSYARLDKQGNFNAWVAGSYGNDQWLQVDLGSSKEVTGIITQGARNFGSVQFVASYKVAYSNDSANWTEYQDPRTGSSKIFPGNWDNHSHKKNLFETPILARYVRILPVAWHNRIALRLELLGC

SEQ ID NO: 11 - DNA, 1936 bp, Homo sapiens
  source 1..1936: mol_type = genomic DNA
agaaccccgcggggtctgagcagcccagcgtgcccattccagcgcccgcgtccccgcagcatgccgcgcccccgcctgctggccgcgctgtgcggcgcgctgctctgcgcccccagcctcctcgtcgccctggatatctgttccaaaaacccctgccacaacggtggtttatgcgaggagatttcccaagaagtgcgaggagatgtcttcccctcgtacacctgcacgtgccttaagggctacgcgggcaaccactgtgagacgaaatgtgtcgagccactgggcatggagaatgggaacattgccaactcacagatcgccgcctcgtctgtgcgtgtgaccttcttgggtttgcagcattgggtcccggagctggcccgcctgaaccgcgcaggcatggtcaatgcctggacacccagcagcaatgacgataacccctggatccaggtgaacctgctgcggaggatgtgggtaacaggtgtggtgacgcagggtgccagccgcttggccagtcatgagtacctgaaggccttcaaggtggcctacagccttaatggacacgaattcgatttcatccatgatgttaataaaaaacacaaggagtttgtgggtaactggaacaaaaacgcggtgcatgtcaacctgtttgagacccctgtggaggctcagtacgtgagattgtaccccacgagctgccacacggcctgcactctgcgctttgagctactgggctgtgagctgaacggatgcgccaatcccctgggcctgaagaataacagcatccctgacaagcagatcacggcctccagcagctacaagacctggggcttgcatctcttcagctggaacccctcctatgcacggctggacaagcagggcaacttcaacgcctgggttgcggggagctacggtaacgatcagtggctgcaggtggacctgggctcctcgaaggaggtgacaggcatcatcacccagggggcccgtaactttggctctgtccagtttgtggcatcctacaaggttgcctacagtaatgacagtgcgaactggactgagtaccaggaccccaggactggcagcagtaagatcttccctggcaactgggacaaccactcccacaagaagaacttgtttgagacgcccatcctggctcgctatgtgcgcatcctgcctgtagcctggcacaaccgcatcgccctgcgcctggagctgctgggctgttagtggccacctgccacccccaggtcttcctgctttccatgggcccgctgcctcttggcttctcagcccctttaaatcaccatagggctggggactggggaaggggagggtgttcagaggcagcaccaccacacagtcacccctccctccctctttcccaccctccacctctcacgggccctgccccagcccctaagccccgtcccctaacccccagtcctcactgtcctgttttcttaggcactgagggatctgagtaggtctgggatggacaggaaagggcaaagtagggcgtgtggtttccctgcccctgtccggaccgccgatcccaggtgcgtgtgtctctgtctctcctagcccctctctcacacatcacattcccatggtggcctcaagaaaggcccggaagcgccaggctggagataacagcctcttgcccgtcggccctgcgtcggccctggggtaccatgtggccacaactgctgtggccccctgtccccaagacacttccccttgtctccctggttgcctctcttgccccttgtcctgaagcccagcgacacagaagggggtggggcgggtctatggggagaaagggagcgaggtcagaggagggcatgggttggcagggtgggcgtttggggccctctatgctggcttttcaccccagaggacacaggcagcttccaaaatatatttatcttcttcacgggaa

SEQ ID NO: 12 - protein, 387 aa, Homo sapiens
MPRPRLLAALCGALLCAPSLLVALDICSKNPCHNGGLCEEISQEVRGDVFPSYTCTCLKGYAGNHCETKCVEPLGMENGNIANSQIAASSVRVTFLGLQHWVPELARLNRAGMVNAWTPSSNDDNPWIQVNLLRRMWVTGVVTQGASRLASHEYLKAFKVAYSLNGHEFDFIHDVNKKHKEFVGNWNKNAVHVNLFETPVEAQYVRLYPTSCHTACTLRFELLGCELNGCANPLGLKNNSIPDKQITASSSYKTWGLHLFSWNPSYARLDKQGNFNAWVAGSYGNDQWLQVDLGSSKEVTGIITQGARNFGSVQFVASYKVAYSNDSANWTEYQDPRTGSSKIFPGNWDNHSHKKNLFETPILARYVRILPVAWHNRIALRLELLGC

SEQ ID NO: 13 - DNA, 2188 bp, Rattus norvegicus
  source 1..2188: mol_type = genomic DNA
attcccctgtgagaggagcggacgcaggaactctccggtcccagcatcggagcttgtggaccatttcccgcgtcccgcagcatgcagttctcccgtgtgctggccgcgctgtgcggtgtgctgctctgcgcctccggcctcttcgctgcgtccggtgacttctgtgactccagcctgtgcctgaatggtgggacctgcttgatgggccaagacaatgacatctactgcctctgccctgaaggcttcacaggccttgtgtgcaacgagactgagaaaggaccgtgttccccaaacccttgcttccacgatgccaaatgcctggtgactgaggacacacagcgaggggacatcttcactgagtacatctgccagtgccctgtgggctactcgggcatccactgtgaactcgagaccacctcctacctggatggagagtacctgtccagcccagccgtccctaccacagccgtccccaccacagccatccccaccacagccgtccccaccacagccgtccccaccacagccgtccccaccccggcccccaaccccgatctttccaaccacctagcctcccgctgttccacaaagctgggcttggaagggggcgccattgccgattcacagatttctgcctcgtctgtgtatatgggcttcatgggcttgcagcgctggggcccggagctggctcgcctgtatcgcacagggattgtcaatgcatggacagccagcagctatgatagcaagccctggatccaggtggactttctgcggaagatgcgggtatcaggtgtgatgacacagggtgccagccgtgccgggagggcggaatacctgaagaccttcaaggtggcttacagcctcgatggacgcaggttcgagttcatccaggatgaaagcggaaccggagacaaggagtttatgggtaaccaggacaacaacagcctgaagattaacatgttcaaccccactctggaggcacagtacataaggctgtaccctgtctcgtgccaccgcggctgcaccctccgcttcgagctcctgggctgcgagttgcatggatgctctgagcccctgggcctgaagaataacacgattcctgacagccaaataacagcctccagcagctacaagacgtggaacctgcgtgcctttggctggtacccccacttggggcggctggacaatcagggcaagatcaatgcctggacagctcagagcaacagtgccaaggaatggctgcaggttgacctgggcactcagaaaaaagtgacaggaattatcacccagggggcccgtgactttggccacatccagtatgtggcatcctataaggtagcccacagtgatgatggtgtgcagtggaccgtatatgaggaacaaggaaccagcaaggtcttccagggcaacttggacaacaactcccacaagaagaacatctttgagaaacctttcatggctcgctatgtgcgtgtccttccactgtcctggcataaccgtatcaccctgcgcctggagctgctgggctgttagtgcccagtccttccagcccaagtgacgaggacggccagaggctgaggggcctcctggccctgcctcccaggccctgctgccttctgtggctgacaccttctcaatcctccctcttgattgcactgggactacaggcaggaagggcaagggggtttcagagttgcccctcacccttcccctcaccctgcagcccccacaggcctcctgctagccccttctctcaggcattctgggggagttggacaggtctgagatgaatagagaagaagagtgaagttggggtatgtgggctgctgtaccaaccaccccaagtcctaaactttctccaggggttgactcaagactaaagggaacctctggttgcccacccgtctctgcacaccgcacatccctccatgttccattcctggaaggagaggcccacgtccgcttgctgccccttgggtcaccagatcctgcctcttatctcctgagacccctcttgaccctcgctctggagcctcggttgacaagaggactgtcgggtctggagagatagatgggctctgggtggttggcgagctggctatgggacctctgctggcttgctacccaagctaacaagcagattccaaaatacatttgtgctctccactggaaaaaaaaaaaaaaaaaa

SEQ ID NO: 14 - protein, 483 aa, Rattus norvegicus
MQFSRVLAALCGVLLCASGLFAASGDFCDSSLCLNGGTCLMGQDNDIYCLCPEGFTGLVCNETEKGPCSPNPCFHDAKCLVTEDTQRGDIFTEYICQCPVGYSGIHCELETTSYLDGEYLSSPAVPTTAVPTTAIPTTAVPTTAVPTTAVPTPAPNPDLSNHLASRCSTKLGLEGGAIADSQISASSVYMGFMGLQRWGPELARLYRTGIVNAWTASSYDSKPWIQVDFLRKMRVSGVMTQGASRAGRAEYLKTFKVAYSLDGRRFEFIQDESGTGDKEFMGNQDNNSLKINMFNPTLEAQYIRLYPVSCHRGCTLRFELLGCELHGCSEPLGLKNNTIPDSQITASSSYKTWNLRAFGWYPHLGRLDNQGKINAWTAQSNSAKEWLQVDLGTQKKVTGIITQGARDFGHIQYVASYKVAHSDDGVQWTVYEEQGTSKVFQGNLDNNSHKKNIFEKPFMARYVRVLPLSWHNRITLRLELLGC

SEQ ID NO: 15 - DNA, 2020 bp, Rattus norvegicus
  source 1..2020: mol_type = genomic DNA
attcccctgtgagaggagcggacgcaggaactctccggtcccagcatcggagcttgtggaccatttcccgcgtcccgcagcatgcagttctcccgtgtgctggccgcgctgtgcggtgtgctgctctgcgcctccggcctcttcgctgcgtccggtgacttctgtgactccagcctgtgcctgaatggtgggacctgcttgatgggccaagacaatgacatctactgcctctgccctgaaggcttcacaggccttgtgtgcaacgagactgagaaaggaccgtgttccccaaacccttgcttccacgatgccaaatgcctggtgactgaggacacacagcgaggggacatcttcactgagtacatctgccagtgccctgtgggctactcgggcatccactgtgaactcggctgttccacaaagctgggcttggaagggggcgccattgccgattcacagatttctgcctcgtctgtgtatatgggcttcatgggcttgcagcgctggggcccggagctggctcgcctgtatcgcacagggattgtcaatgcatggacagccagcagctatgatagcaagccctggatccaggtggactttctgcggaagatgcgggtatcaggtgtgatgacacagggtgccagccgtgccgggagggcggaatacctgaagaccttcaaggtggcttacagcctcgatggacgcaggttcgagttcatccaggatgaaagcggaaccggagacaaggagtttatgggtaaccaggacaacaacagcctgaagattaacatgttcaaccccactctggaggcacagtacataaggctgtaccctgtctcgtgccaccgcggctgcaccctccgcttcgagctcctgggctgcgagttgcatggatgctctgagcccctgggcctgaagaataacacgattcctgacagccaaataacagcctccagcagctacaagacgtggaacctgcgtgcctttggctggtacccccacttggggcggctggacaatcagggcaagatcaatgcctggacagctcagagcaacagtgccaaggaatggctgcaggttgacctgggcactcagaaaaaagtgacaggaattatcacccagggggcccgtgactttggccacatccagtatgtggcatcctataaggtagcccacagtgatgatggtgtgcagtggaccgtatatgaggaacaaggaaccagcaaggtcttccagggcaacttggacaacaactcccacaagaagaacatctttgagaaacctttcatggctcgctatgtgcgtgtccttccactgtcctggcataaccgtatcaccctgcgcctggagctgctgggctgttagtgcccagtccttccagcccaagtgacgaggacggccagaggctgaggggcctcctggccctgcctcccaggccctgctgccttctgtggctgacaccttctcaatcctccctcttgattgcactgggactacaggcaggaagggcaagggggtttcagagttgcccctcacccttcccctcaccctgcagcccccacaggcctcctgctagccccttctctcaggcattctgggggagttggacaggtctgagatgaatagagaagaagagtgaagttggggtatgtgggctgctgtaccaaccaccccaagtcctaaactttctccaggggttgactcaagactaaagggaacctctggttgcccacccgtctctgcacaccgcacatccctccatgttccattcctggaaggagaggcccacgtccgcttgctgccccttgggtcaccagatcctgcctcttatctcctgagacccctcttgaccctcgctctggagcctcggttgacaagaggactgtcgggtctggagagatagatgggctctgggtggttggcgagctggctatgggacctctgctggcttgctacccaagctaacaagcagattccaaaatacatttgtgctctccactggaaaaaaaaaaaaaaaaaa

SEQ ID NO: 16 - protein, 427 aa, Rattus norvegicus
MQFSRVLAALCGVLLCASGLFAASGDFCDSSLCLNGGTCLMGQDNDIYCLCPEGFTGLVCNETEKGPCSPNPCFHDAKCLVTEDTQRGDIFTEYICQCPVGYSGIHCELGCSTKLGLEGGAIADSQISASSVYMGFMGLQRWGPELARLYRTGIVNAWTASSYDSKPWIQVDFLRKMRVSGVMTQGASRAGRAEYLKTFKVAYSLDGRRFEFIQDESGTGDKEFMGNQDNNSLKINMFNPTLEAQYIRLYPVSCHRGCTLRFELLGCELHGCSEPLGLKNNTIPDSQITASSSYKTWNLRAFGWYPHLGRLDNQGKINAWTAQSNSAKEWLQVDLGTQKKVTGIITQGARDFGHIQYVASYKVAHSDDGVQWTVYEEQGTSKVFQGNLDNNSHKKNIFEKPFMARYVRVLPLSWHNRITLRLELLGC

SEQ ID NO: 17 - DNA, 2032 bp, Mus musculus
  source 1..2032: mol_type = genomic DNA
ggcgcctgatttattccggagtgagaggagcggacgcaggaactctcgagtcccagcatcagagcgcgtggaccttttcccgcgtcccgcagcatgcaggtctcccgtgtgctggccgcgctgtgcggcatgctactctgcgcctctggcctcttcgccgcgtctggtgacttctgtgactccagcctgtgcctgaacggtggcacctgcttgacgggccaagacaatgacatctactgcctctgccctgaaggcttcacaggccttgtgtgcaatgagactgagagaggaccatgctccccaaacccttgctacaatgatgccaaatgtctggtgactttggacacacagcgtggggacatcttcaccgaatacatctgccagtgccctgtgggctactcgggcatccactgtgaaaccggttgttctacacagctgggcatggaagggggcgccattgctgattcacagatttccgcctcgtctgtgtatatgggtttcatgggcttgcagcgctggggcccggagctggctcgtctgtaccgcacagggatcgtcaatgcctggacagccagcaactatgatagcaagccctggatccaggtgaaccttctgcggaagatgcgggtatcaggtgtgatgacgcagggtgccagccgtgccgggagggcggagtacctgaagaccttcaaggtggcttacagcctcgacggacgcaagtttgagttcatccaggatgaaagcggtggagacaaggagtttttgggtaacctggacaacaacagcctgaaggttaacatgttcaacccgactctggaggcacagtacataaagctgtaccctgtttcgtgccaccgcggctgcaccctccgcttcgagctcctgggctgtgagttgcacggatgttctgagcccctgggcctgaagaataacacaattcctgacagccagatgtcagcctccagcagctacaagacatggaacctgcgtgcttttggctggtacccccacttgggaaggctggataatcagggcaagatcaatgcctggacggctcagagcaacagtgccaaggaatggctgcaggttgacctgggcactcagaggcaagtgacaggaatcatcacccagggggcccgtgactttggccacatccagtatgtggcgtcctacaaggtagcccacagtgatgatggtgtgcagtggactgtatatgaggagcaaggaagcagcaaggtcttccagggcaacttggacaacaactcccacaagaagaacatcttcgagaaacccttcatggctcgctacgtgcgtgtccttccagtgtcctggcataaccgcatcaccctgcgcctggagctgctgggctgttaatgctcagtcctgccagcccaaacgatgaggatggccagaggctgaggggcctcctggccctgcctcccaggccctgctgccttctgtggctgacgaccttcttggccttcccttctgattgtactggggctggaggcaggaagggccaggggatttcagagttgcccttcaccctttccctcaccctgcagcccccacaggcctcctgctagcccccttctctcaggcattctgggggagttggacaggtctgagatgaatagagaagaagagtgaagttggggtatgtgggctatctgtaccaaccaccccaagtcctaaacttcctgccagggcttgactcaggactgaagggagcccctgactgcccatccctctctgcacaccacacattcctccatgttccattccgggaaggagaggcccacgtccgcttgctgtcccttgggtcaccaggtcctgcctcttatctcctgagacgcctcttgacccttgcactggagcctcagttgacaaggagactggcgggtctggagaggtcggtggctctgggtggttgacaggttggctgtgggacctctgctggcttgctacccaagttaacaagcagattccaaaatacattcgtgttctccactggaaaaaaaaaaaaaaaaaa

SEQ ID NO: 18 - protein, 426 aa, Mus musculus
MQVSRVLAALCGMLLCASGLFAASGDFCDSSLCLNGGTCLTGQDNDIYCLCPEGFTGLVCNETERGPCSPNPCYNDAKCLVTLDTQRGDIFTEYICQCPVGYSGIHCETGCSTQLGMEGGAIADSQISASSVYMGFMGLQRWGPELARLYRTGIVNAWTASNYDSKPWIQVNLLRKMRVSGVMTQGASRAGRAEYLKTFKVAYSLDGRKFEFIQDESGGDKEFLGNLDNNSLKVNMFNPTLEAQYIKLYPVSCHRGCTLRFELLGCELHGCSEPLGLKNNTIPDSQMSASSSYKTWNLRAFGWYPHLGRLDNQGKINAWTAQSNSAKEWLQVDLGTQRQVTGIITQGARDFGHIQYVASYKVAHSDDGVQWTVYEEQGSSKVFQGNLDNNSHKKNIFEKPFMARYVRVLPVSWHNRITLRLELLGC

SEQ ID NO: 19 - DNA, 2143 bp, Mus musculus
  source 1..2143: mol_type = genomic DNA
ggcgcctgatttattccggagtgagaggagcggacgcaggaactctcgagtcccagcatcagagcgcgtggaccttttcccgcgtcccgcagcatgcaggtctcccgtgtgctggccgcgctgtgcggcatgctactctgcgcctctggcctcttcgccgcgtctggtgacttctgtgactccagcctgtgcctgaacggtggcacctgcttgacgggccaagacaatgacatctactgcctctgccctgaaggcttcacaggccttgtgtgcaatgagactgagagaggaccatgctccccaaacccttgctacaatgatgccaaatgtctggtgactttggacacacagcgtggggacatcttcaccgaatacatctgccagtgccctgtgggctactcgggcatccactgtgaaaccgagaccaactactacaacctggatggagaatacatgttcaccacagccgtccccaatactgccgtccccaccccggcccccacccccgatctttccaacaacctagcctcccgttgttctacacagctgggcatggaagggggcgccattgctgattcacagatttccgcctcgtctgtgtatatgggtttcatgggcttgcagcgctggggcccggagctggctcgtctgtaccgcacagggatcgtcaatgcctggacagccagcaactatgatagcaagccctggatccaggtgaaccttctgcggaagatgcgggtatcaggtgtgatgacgcagggtgccagccgtgccgggagggcggagtacctgaagaccttcaaggtggcttacagcctcgacggacgcaagtttgagttcatccaggatgaaagcggtggagacaaggagtttttgggtaacctggacaacaacagcctgaaggttaacatgttcaacccgactctggaggcacagtacataaagctgtaccctgtttcgtgccaccgcggctgcaccctccgcttcgagctcctgggctgtgagttgcacggatgttctgagcccctgggcctgaagaataacacaattcctgacagccagatgtcagcctccagcagctacaagacatggaacctgcgtgcttttggctggtacccccacttgggaaggctggataatcagggcaagatcaatgcctggacggctcagagcaacagtgccaaggaatggctgcaggttgacctgggcactcagaggcaagtgacaggaatcatcacccagggggcccgtgactttggccacatccagtatgtggcgtcctacaaggtagcccacagtgatgatggtgtgcagtggactgtatatgaggagcaaggaagcagcaaggtcttccagggcaacttggacaacaactcccacaagaagaacatcttcgagaaacccttcatggctcgctacgtgcgtgtccttccagtgtcctggcataaccgcatcaccctgcgcctggagctgctgggctgttaatgctcagtcctgccagcccaaacgatgaggatggccagaggctgaggggcctcctggccctgcctcccaggccctgctgccttctgtggctgacgaccttcttggccttcccttctgattgtactggggctggaggcaggaagggccaggggatttcagagttgcccttcaccctttccctcaccctgcagcccccacaggcctcctgctagcccccttctctcaggcattctgggggagttggacaggtctgagatgaatagagaagaagagtgaagttggggtatgtgggctatctgtaccaaccaccccaagtcctaaacttcctgccagggcttgactcaggactgaagggagcccctgactgcccatccctctctgcacaccacacattcctccatgttccattccgggaaggagaggcccacgtccgcttgctgtcccttgggtcaccaggtcctgcctcttatctcctgagacgcctcttgacccttgcactggagcctcagttgacaaggagactggcgggtctggagaggtcggtggctctgggtggttgacaggttggctgtgggacctctgctggcttgctacccaagttaacaagcagattccaaaatacattcgtgttctccactggaaaaaaaaaaaaaaaaaa

SEQ ID NO: 20 - protein, 463 aa, Mus musculus
MQVSRVLAALCGMLLCASGLFAASGDFCDSSLCLNGGTCLTGQDNDIYCLCPEGFTGLVCNETERGPCSPNPCYNDAKCLVTLDTQRGDIFTEYICQCPVGYSGIHCETETNYYNLDGEYMFTTAVPNTAVPTPAPTPDLSNNLASRCSTQLGMEGGAIADSQISASSVYMGFMGLQRWGPELARLYRTGIVNAWTASNYDSKPWIQVNLLRKMRVSGVMTQGASRAGRAEYLKTFKVAYSLDGRKFEFIQDESGGDKEFLGNLDNNSLKVNMFNPTLEAQYIKLYPVSCHRGCTLRFELLGCELHGCSEPLGLKNNTIPDSQMSASSSYKTWNLRAFGWYPHLGRLDNQGKINAWTAQSNSAKEWLQVDLGTQRQVTGIITQGARDFGHIQYVASYKVAHSDDGVQWTVYEEQGSSKVFQGNLDNNSHKKNIFEKPFMARYVRVLPVSWHNRITLRLELLGC

SEQ ID NO: 21 - protein, 6 aa, synthetic construct
  REGION 1..6: note = Description of Artificial Sequence: Synthetic 6xHis tag
HHHHHH

SEQ ID NO: 22 - DNA, 19 bp, synthetic construct
  misc_feature 1..19: note = Description of Artificial Sequence: Synthetic primer
  source 1..19: mol_type = other DNA
gttgtctcctgcgacttca

SEQ ID NO: 23 - DNA, 19 bp, synthetic construct
  misc_feature 1..19: note = Description of Artificial Sequence: Synthetic primer
  source 1..19: mol_type = other DNA
ggtggtccagggtttctta

SEQ ID NO: 24 - DNA, 24 bp, synthetic construct
  misc_feature 1..24: note = Description of Artificial Sequence: Synthetic primer
  source 1..24: mol_type = other DNA
caatgcaatgaagaactggactgt

SEQ ID NO: 25 - DNA, 24 bp, synthetic construct
  misc_feature 1..24: note = Description of Artificial Sequence: Synthetic primer
  source 1..24: mol_type = other DNA
tcctacatcttctgagtttggtga

SEQ ID NO: 26 - DNA, 20 bp, synthetic construct
  misc_feature 1..20: note = Description of Artificial Sequence: Synthetic primer
  source 1..20: mol_type = other DNA
gcagggttccaacgatgttg

SEQ ID NO: 27 - DNA, 21 bp, synthetic construct
  misc_feature 1..21: note = Description of Artificial Sequence: Synthetic primer
  source 1..21: mol_type = other DNA
gcagccatcgactaggacaga

SEQ ID NO: 28 - DNA, 20 bp, synthetic construct
  misc_feature 1..20: note = Description of Artificial Sequence: Synthetic primer
  source 1..20: mol_type = other DNA
ctgacagaggcaccactgaa

SEQ ID NO: 29 - DNA, 20 bp, synthetic construct
  misc_feature 1..20: note = Description of Artificial Sequence: Synthetic primer
  source 1..20: mol_type = other DNA
catctccagagtccagcaca

SEQ ID NO: 30 - DNA, 22 bp, synthetic construct
  misc_feature 1..22: note = Description of Artificial Sequence: Synthetic primer
  source 1..22: mol_type = other DNA
tgacgtcactggagttgtacgg

SEQ ID NO: 31 - DNA, 21 bp, synthetic construct
  misc_feature 1..21: note = Description of Artificial Sequence: Synthetic primer
  source 1..21: mol_type = other DNA
ggttcatgtcatggatggtgc

SEQ ID NO: 32 - DNA, 22 bp, synthetic construct
  misc_feature 1..22: note = Description of Artificial Sequence: Synthetic primer
  source 1..22: mol_type = other DNA
aactttgagaaggatggcaagt

SEQ ID NO: 33 - DNA, 19 bp, synthetic construct
  misc_feature 1..19: note = Description of Artificial Sequence: Synthetic primer
  source 1..19: mol_type = other DNA
tgccacccatggtaaacaa

SEQ ID NO: 34 - DNA, 21 bp, synthetic construct
  misc_feature 1..21: note = Description of Artificial Sequence: Synthetic primer
  source 1..21: mol_type = other DNA
ctggacagccagacactaaag

SEQ ID NO: 35 - DNA, 20 bp, synthetic construct
  misc_feature 1..20: note = Description of Artificial Sequence: Synthetic primer
  source 1..20: mol_type = other DNA
ctcgcggcaagtcttcagag

SEQ ID NO: 36 - DNA, 20 bp, synthetic construct
  misc_feature 1..20: note = Description of Artificial Sequence: Synthetic primer
  source 1..20: mol_type = other DNA
gctctctgctcctcctgttc

SEQ ID NO: 37 - DNA, 20 bp, synthetic construct
  misc_feature 1..20: note = Description of Artificial Sequence: Synthetic primer
  source 1..20: mol_type = other DNA
ccatggtgtctgagcgatgt

SEQ ID NO: 38 - DNA, 21 bp, synthetic construct
  misc_feature 1..21: note = Description of Artificial Sequence: Synthetic primer
  source 1..21: mol_type = other DNA
agctcccggaaaagattgatg

SEQ ID NO: 39 - DNA, 21 bp, synthetic construct
  misc_feature 1..21: note = Description of Artificial Sequence: Synthetic primer
  source 1..21: mol_type = other DNA
cagggtgctggctgagtagat

SEQ ID NO: 40 - DNA, 18 bp, synthetic construct
  misc_feature 1..18: note = Description of Artificial Sequence: Synthetic primer
  source 1..18: mol_type = other DNA
cacgcacgacgtcttcca

SEQ ID NO: 41 - DNA, 19 bp, synthetic construct
  misc_feature 1..19: note = Description of Artificial Sequence: Synthetic primer
  source 1..19: mol_type = other DNA
aagcggtcctggcagaaat